(19) United States
(12) Patent Application Publication
(10) Pub. No.: US 2011/0275094 A1
(43) Pub. Date: Nov. 10, 2011

(54) PHOSPHORYLATED FMS-RELATED TYROSINE KINASE 3 BIOMARKER ASSAY

(76) Inventor: Ruwanthi N. Gunawardane, Seattle, WA (US)

(21) Appl. No.: 13/127,856

(22) PCT Filed: Nov. 6, 2009
(86) PCT No.: PCT/US09/63534

§ 371 Date: Jun. 7, 2011

Related U.S. Application Data
(60) Provisional application No. 61/112,110, filed on Nov. 6, 2008.
(60) Provisional application No. 61/120,325, filed on Dec. 5, 2008.

(51) Int. Cl. G01N 33/573; G01N 33/577

(52) U.S. Cl. 435/7.5; 435/7.1; 435/7.94

(57) ABSTRACT

Provided herein are methods for detecting the presence of human pFLT3 in a sample. An exemplary assay provided herein is an ELISA method (e.g., sandwich ELISA). Also provided herein are methods for detecting FLT3 phosphorylation in a sample, diagnosing a patient having a FLT3-activating mutation, identifying a compound that activates or is otherwise and agonist of human FLT3 phosphorylation, identifying a compound that inhibits or is otherwise an antagonist of human FLT3 phosphorylation, determining the efficacy of a compound for increasing, decreasing or otherwise modulating human FLT3 phosphorylation in a patient. Further provided are kits for carrying out the said methods. Such kits comprise at least a total FLT3 antibody optionally immobilized on a solid support and a labeled pFLT3 antibody.

### FIELD

Provided herein are methods for detecting the presence of human pFLT3 in a sample, such as human blood. An exemplary assay provided herein is an enzyme-linked immunosorbent assay (ELISA), for example, a sandwich ELISA. Also provided herein are methods for detecting FLT3 phosphorylation in a sample. Further provided are methods for diagnosing a patient having a FLT3-activating mutation. Also provided are methods for identifying a compound that activates or inhibits human FLT3 phosphorylation. Further provided are methods for determining the efficacy of a compound for increasing, decreasing or otherwise modulating human FLT3 phosphorylation in a patient. Also provided are kits for carrying out said methods.

### BACKGROUND

FMS-related tyrosine kinase 3 (FLT3) (also known as FMS-like tyrosine kinase 3, fetal liver kinase 2 (FLK-2), stem cell kinase 1 (STK-1), and cluster of differentiation 135 (CD135)) is a membrane-bound receptor tyrosine kinase that plays an important role in the proliferation and differentiation of hematopoietic stem cells. The gene was first cloned from a mouse gene (Rosnet et al. (1991) Oncogene 6:1641-1650, Matthews et al. Cell 1991 65:1143-1152), which was followed by the identification of the human FLT3 gene (Small et al. (1994) Proc. Natl. Acad. Sci. USA 91:459-463).

FLT3 is expressed in normal myeloid and lymphoid progenitors, and activation by FLT3-ligand (FLT3-L) leads to the growth and differentiation of hematopoietic progenitor cells. When bound to FLT3-L, FLT3 forms a homodimer with itself, which promotes phosphorylation of the receptor and activation of downstream cell signaling. FLT3 is also expressed in leukemic cells as wild type or mutant FLT3 in a number of hematological malignancies, including acute myeloid leukemia (AML), acute promyelocytic leukemia (APL), acute lymphoblastic leukemia (ALL), myelodysplasic syndrome (MDS) and trilineage myelodysplasic syndrome (TMDS). FLT3 is also found on cells from patients with chronic myelogenous leukemia in lymphoid blast crisis.

The most common FLT3 mutation to occur among hematological malignancies is the FLT3-ITD mutations. FLT3-ITD mutations display ligand-independent receptor dimerization that results in autophosphorylation and constitutive activation of the FLT3 receptor which, in turn, leads to ligand-independent proliferation of early hematopoietic cells with loss of differentiation. (Hayakawa (2000) Oncogene 19:624-631). The second most common FLT3 mutation is a mutation in the tyrosine kinase domain (TKD). Patients having the FLT3-ITD mutation generally have a poorer prognosis compared to wild-type patients, and therefore the FLT3-ITD mutation has been a target for molecular-based therapy. (Stirewalt et al. (2003)Nat. Rev. Cancer (2003) 3:650, Levis et al. (2005)Int. J. Hematol. 82:100-107).

Small molecule FLT3 inhibitors such as SU5416, CEP-701, PKC412 and MLN518 have been studied in clinical trials with patients showing clinical response in the form of reduction in bone marrow blast or peripheral blast. (Levis et al. (2005) Int J Hematol. 82: 100-107). In clinical studies using these FLT3 inhibitors, levels of pFLT3 were measured as a pharmacodynamic (PD) marker to determine a patient's response to the inhibitors at the molecular level, by immunoprecipitating FLT3 protein from patient plasma samples, immunoblotting (e.g., IP/Western), and then detecting pFLT3 with an anti-phosphotyrosine antibody (Smith et al. (2004) Blood 103:3669-3676, Brown et al. (2006) Leukemia 20:1368-1376, De Angelo et al. (2006) Blood 108:3674-3681, Stone et al. (2005) Blood 105:54-60, Knapper et al. (2006) Blood 108(10): 3494-3503). In a typical setting, these methods require immunoprecipitation of at least about 20 million cells to produce a good signal, and involves processing over the course of several days a number of small sets of samples. Therefore, some of the shortcomings of the immunoprecipitation/immunoblotting method are its high sample protein requirement, its lack of sensitivity, the qualitative nature of the data and the time consuming and laborious nature of the assay, all of which make it unsuitable for diagnostic or clinical monitoring purposes. Because of its lack of sensitivity in the immunblotting method, pFLT3 can be detected only in those leukemia patients that express a high enough level of pFLT3 in plasma, and thus far, pFLT3 has been reported only for select patients expressing sufficiently high levels of pFLT3 in plasma. In another variation of the immunoprecipitation method, FLT3 in patient plasma samples has been immunoprecipitated, and both total FLT3 and pFLT3 has been detected by flow cytometry (FACS). (Ravandi et al. (2007) Leuk. Res. 31:791-797, Zheng et al. (2004) Blood 103:267-274). Detection by flow cytometry also suffers from the same drawbacks of being semi-quantitative, time-consuming, expensive and laborious.

Given the number of FLT3 inhibitors in the clinic, and given the low levels of pFLT3 expressed in in vivo tissue samples, there remains a need for a sensitive and efficient method of determining the level of pFLT3 inhibition as a pharmacodynamic marker in patients receiving FLT3 inhibitors or other targeted therapy that inhibit the FLT3 activation pathway. Such a method for monitoring pFLT3 could be done over the course of treatment to detect, for example, early response to FLT3 therapy. Because early response to therapy may be an indication of chemosensitivity, pFLT3 inhibition could prove to be another prognostic indicator that could help further define treatment strategies (Kern et al. Blood 2003 101(1), 64-70). For example, rapidity of response to induction chemotherapy is emerging as an important prognostic factor in leukemias such as childhood ALL. There is a likelihood that rapidity of response to targeted therapy may also emerge as an important prognostic factor in leukemia. Phosphorylated FLT3 levels may be monitored as a pharmacodynamic marker for response to either induction chemotherapy or targeted therapy and may be used in turn to stratify patients further based on rapidity of response Plasma pFLT3 levels could also be used as a diagnostic or prognostic biomarker for a patient. For example, as discussed elsewhere herein, there is also a strong correlation between high pFLT3 levels and FLT3-ITD status as determined by genotyping.

Accordingly, there is a need to develop a timely and cost-effective method that can be performed consistently in AML and other leukemic diagnoses. Since phosphorylation and FLT3-activating mutations, such as FLT3-ITD, are associated with AML, methods enabling specific detection of FLT3 activity would have broad applications in clinical use and pharmaceutical research for AML and other FLT3-mediated diseases.

### SUMMARY

Provided herein are methods for detecting the presence of human pFLT3 in a sample. An exemplary assay provided herein is an enzyme-linked immunosorbent assay (ELISA), for example, a sandwich ELISA. Also provided herein are methods for detecting FLT3 phosphorylation in a sample. Further provided are methods for diagnosing a patient having a FLT3-activating mutation. Also provided are methods for identifying a compound that activates or is otherwise an agonist of human FLT3 phosphorylation. Further provided are methods for identifying a compound that inhibits or is otherwise and antagonist of human FLT3 phosphorylation. Also provided are methods for determining the efficacy of a compound for decreasing or otherwise modulating human FLT3 phosphorylation in a patient. Also provided are kits for carrying out said methods.

Thus, in one aspect, provided herein is a method for detecting the presence of human pFLT3 in a sample, e.g., an in vivo sample, said method comprising (a) contacting the sample with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (d) removing unbound second antibody; and (e) detecting the presence of second antibody bound to the sample; wherein detection above background of an amount of the second antibody bound to the sample, e.g., an increase in the amount of second antibody bound to the sample, as compared to a control sample having no pFLT3, indicates the presence of human pFLT3 in the sample. In certain embodiments, the sample is blood from a human patient, lysate of human blood or bone marrow from a patient.

In a second aspect, provided herein is a method for detecting the presence of human pFLT3 in a sample, e.g., an in vivo sample, said method comprising: (a) contacting the sample with an immobilized first antibody that immunospecifically binds to the extracellular domain of human FLT3 (27-543 of SEQ ID NO:1), wherein the first antibody is immobilized on a multi-well plate or multi-domain multi-well plate comprising carbon ink electrodes, such as on the bottom of the plate (e.g., MULTI-ARRAY® plates (Meso Scale Discovery, Gaithersburg, Md.)); (b) removing unbound sample; (c) contacting the sample bound to the immobilized first antibody with a biotinylated second antibody, wherein the second antibody immunospecifically binds to phosphotyrosine (i.e., an anti-phosphotyrosine antibody); (d) removing unbound second antibody; (e) contacting the biotinylated second antibody with labeled-streptavidin, wherein the label comprises ruthenium (II) tri-bipyridine-(4-methylsulfonate) NHS ester (e.g., SULFO-TAG™ (Meso Scale Discovery, Gaithersburg, Md.)); (f) removing unbound labeled-streptavidin; and (g) detecting the presence of second antibody bound to the sample by electrochemiluminescence (ECL) of the labeled-streptavidin on the surface of the carbon electrodes; wherein detection above background of an amount of the second antibody bound to the sample, e.g., an increase in the amount of second antibody bound to the sample, as compared to a control sample having no pFLT3, indicates the presence of human pFLT3 in the sample. In certain embodiments, the sample is blood from a human patient, lysate of human blood or bone marrow from a patient.

In a third aspect, provided herein are methods for diagnosing a patient having a FLT3-activating mutation, said method comprising: (a) contacting a sample from the patient with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (d) removing unbound second antibody; and (e) detecting or otherwise measuring the presence of second antibody bound to the sample. In certain embodiments, detection above background of an amount of the second antibody bound to the sample, e.g., an increase in the amount of second antibody bound to the sample, as compared to a second sample lacking pFLT3, correlates with the presence of the FLT3-activating mutation in the patient. In certain embodiments, the amount of second antibody detected corresponds to a greater amount of pFLT3 in the sample than in a corresponding sample from, e.g., a patient or cell line that does not have a FLT3-activating mutation. In some embodiments, the method further comprises (f) comparing the amount of second antibody detected to a probability table and assigning a probability of the sample containing a FLT3-activating mutation to the amount of detected second antibody, whereby the patient is diagnosed as having a FLT3-activating mutation. In one embodiment, the FLT3-activating mutation is a FLT3-ITD mutation. In certain embodiments, the sample is blood from a human patient, lysate of human blood or bone marrow from a patient. Thus, in certain embodiments, provided herein are methods for diagnosing a patient having a FLT3-activating mutation, e.g., a FLT3-ITD mutation, comprising detecting the amount of pFLT3 present in an in vivo sample of the patient (such as blood, blood lysate or bone marrow), and identifying the amount of pFLT3 present as corresponding to an amount of pFLT3 present in a sample having a FLT3-activating mutation, e.g., a FLT3-ITD mutation. In certain embodiments, the determination is performed using a sample directly from the patient, that is, without any sample processing beyond cell lysis prior to the method being performed.

In a fourth aspect, provided herein are methods for identifying a test compound that activates human FLT3 phosphorylation or is otherwise a FLT3 agonist, said method comprising: (a) contacting a sample comprising human FLT3 in the presence and absence of test compound; (b) contacting the sample with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein an increase in the amount of second antibody bound to the sample in the presence of the test compound, as compared to the amount of second antibody bound to the sample in the absence of the test compound, indicates that the test compound activates human FLT3 phosphorylation. In one embodiment, the human FLT3 phosphorylation is the result of a FLT3-activating mutation, such as a FLT3-ITD mutation. In certain embodiments, the test compound is one of a plurality of test compounds, wherein at lest two of the test compounds differ from one another. In some embodiments, the plurality of test compounds comprises between 1 and 100,000 test compounds. In certain embodiments, the sample is blood from a human patient, lysate of human blood or bone marrow from a patient.

In a fifth aspect, provided herein are methods for identifying a test compound that inhibits human FLT3 phosphorylation or is otherwise a FLT3 antagonist, said method comprising: (a) contacting a sample comprising human FLT3 in the presence and absence of test compound; (b) contacting the sample with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein an decrease in the amount of second antibody bound to the sample in the presence of the test compound, as compared to the amount of second antibody bound to the sample in the absence of the test compound, indicates the test compound inhibits human FLT3 phosphorylation. In one embodiment, the human FLT3 phosphorylation is the result of a FLT3-activating mutation, such as a FLT3-ITD mutation. In certain embodiments, the test compound is one of a plurality of test compounds, wherein at lest two of the test compounds differ from one another. In some embodiments, the plurality of test compounds comprises between 1 and 100,000 test compounds. In certain embodiments, the sample is blood from a human patient, lysate of human blood or bone marrow from a patient.

In a sixth aspect, provided herein are methods for determining or monitoring the efficacy of a compound for inhibiting or decreasing human FLT3 phosphorylation in a patient, said method comprising: (a) administering the compound to the patient; (b) contacting a sample from the patient with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein a decrease in the amount of second antibody bound to the sample, as compared to a second sample from the patient from a different (earlier or later) time point, such as prior to administration of the compound, indicates the efficacy of the compound for decreasing human FLT3 phosphorylation in the patient. In other embodiments, the samples are run sequentially at different times. In one embodiment, the human FLT3 phosphorylation is the result of a FLT3-activating mutation, such as a FLT3-ITD mutation. In certain embodiments, the sample is blood from a human patient, lysate of human blood or bone marrow from a patient.

In a seventh aspect, provided herein are methods of preventing, treating, or otherwise managing a patient's FLT3-mediated disease or symptom thereof, said method comprising: (a) administering a compound or other therapy to the patient; (b) contacting a sample from the patient with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein a decrease in the amount of second antibody bound to the sample, as compared to a sample from the patient prior to administration of the compound, indicates the compound prevents, treats, or otherwise manages human FLT3 phosphorylation in the patient. In one embodiment, the human FLT3 phosphorylation is the result of a FLT3-activating mutation, such as a FLT3-ITD mutation. In certain embodiments, the sample is blood from a human patient, lysate of human blood or bone marrow from a patient. In another embodiment, the sample is bone marrow or other tissue. In some embodiments, the method of decreasing human FLT3 phosphorylation is used to prevent, treat or otherwise manage a FLT3-mediated disease or disorder or a symptom thereof.

In one embodiment, provided herein are methods of preventing and/or managing a patient's FLT3-mediated disease or symptom thereof, said method comprising: (a) administering a compound to the patient; (b) contacting a sample from the patient with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample. In certain embodiments, the method further comprises (g) comparing the amount of second antibody detected in a sample taken from the patient prior to step (a) or step (b), whereby the patient continues treatment if there is a decrease in the amount of second antibody detected in step (f) and whereby the patient ceases treatment if there is an increase in the amount of antibody detected in step (f). In one embodiment, the human FLT3 phosphorylation is the result of a FLT3-activating mutation, such as a FLT3-ITD mutation. In certain embodiments, the sample is blood from a human patient, lysate of human blood or bone marrow from a patient. In another embodiment, the sample is bone marrow or other tissue. In some embodiments, the method of decreasing human FLT3 phosphorylation is used to prevent, treat or otherwise manage a FLT3-mediated disease or disorder or a symptom thereof.

In an eighth aspect, provided herein are methods for diagnosing a FLT3-mediated hematological malignancy, said method comprising: (a) contacting a sample, e.g., an in vivo sample (such as blood, blood lysate, plasma or bone marrow), from the patient with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (d) removing unbound second antibody; and (e) detecting or otherwise measuring the presence of second antibody bound to the sample. In certain embodiments, the amount of second antibody detected corresponds to a greater amount of pFLT3 in the sample than in a corresponding sample from a subject or patient that does not have the FLT3-mediated hematological malignancy, correlates with the presence of the FLT3-mediated hematological malignancy. In some embodiments, the method further comprises (f) comparing the amount of second antibody detected to a probability table and assigning a probability of the patient having a FLT3-mediated hematological malignancy to the amount of detected second antibody, whereby the patient is diagnosed with or not diagnosed with the hematological malignancy.

In a ninth aspect, provided herein are methods for diagnosing a patient with high blast count, said method comprising: (a) contacting a sample, e.g., an in vivo sample (such as blood, blood lysate, plasma or bone marrow), from the patient with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (d) removing unbound second antibody; and (e) detecting the presence of second antibody bound to the sample. In certain embodiments, the method further comprises (f) comparing the amount of second antibody detected to a correlation table assigning the amount of detected second antibody to a specific range of blast counts whereby the patient is diagnosed as having a blast count within the range. Thus, in certain embodiments, provided herein are methods for diagnosing a patient with high blast count comprising: determining the amount of pFLT3 present in an in vivo sample of the patient (such as blood, blood lysate or bone marrow), and identifying the amount of pFLT3 present in the sample as corresponding to an amount of pFLT3 indicative of a specific range of blast cells being present in the sample. In certain embodiments, the determination is performed using a sample directly from the patient, that is, without any sample processing beyond cell lysis prior to the method being performed.

In a tenth aspect, provided herein are kits for detecting human pFLT3 in an in vivo sample, comprising: (a) a first antibody that immunospecifically binds to human total FLT3, optionally immobilized on a solid surface; (b) a detectable second antibody, wherein the second antibody immunospecifically binds to human pFLT3, and wherein the second antibody immunospecifically binds to a different FLT3 epitope than the first antibody (such as an epitope comprising a phosphotyrosine). In another embodiment, the kit further comprises an anti-human FLT3 antibody that immunospecifically binds to human FLT3 in a region or epitope that is accessible when the human FLT3 is already bound to the first antibody. In certain embodiments, the in vivo sample is blood from a human patient, lysate of human blood or bone marrow from a patient. In some embodiments, the sample is blood from a human patient having ACL, ALL or another disease or disorder resulting from a FLT3-activating mutation, such as a FLT3-ITD mutation. In some embodiments, the first antibody is immobilized on a multi-well plate comprising carbon electrodes (e.g., MULTI-ARRAY® (Meso Scale Discovery, Gaithersburg, Md.)), the second antibody is a biotinylated anti-phosphotyrosine antibody, and/or the kit further comprises a labeled-streptavidin, wherein the label comprises ruthenium (II) tri-bipyridine-(4-methylsulfonate) NHS ester (e.g., SULFO-TAG™ (Meso Scale Discovery, Gaithersburg, Md.)).

It is noted that in any aspect presented herein, the methods can comprise detecting, for example, simultaneously detecting, both phosphorylated FLT3 (pFLT3) and total FLT3 (tFLT3) in a sample, e.g., an in vivo sample. For example, a method for detecting human pFLT3 in a sample can further comprise also detecting human tFLT3 in the same. In one embodiment, such a method can comprise: (a) contacting in a first and second vessel, e.g., well, such as microtiter well, the sample with an immobilized first antibody that immunospecifically binds to human total FLT3; (b) removing unbound sample; (c) contacting the sample remaining in the first vessel with a detectable second antibody that immunospecifically binds to human total FLT3, wherein the second antibody immunospecifically binds to a different total FLT3 epitope than the first antibody; (d) contacting the sample remaining in the second vessel with a detectable third antibody that immunospecifically binds to human pFLT3, wherein the third antibody binds to a different FLT3 epitope than the first or second antibody (such as an epitope comprising a phosphotyrosine); (e) removing unbound second and third antibodies; and (f) detecting the presence of the second antibody as an indication of the presence of total FLT3 in the sample; and detecting the presence of third antibody as an indication of the presence of pFLT3 in the sample. As with all aspects presented herein, detection can be qualitative or quantitative. Detection and measurement of total FLT3 in a sample can be used, for example, as a control, e.g., a control to verify that total FLT3 was “captured” by the first antibody, or can, for example, be used in normalizing pFLT3 levels obtains in the methods.

### TERMINOLOGY

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

The term “about” or “approximately” means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 20%, 15%, 10%, 9%, 8%, 7%, 6%, 5%, 4%, 3%, 2%, 1%, 0.5%, or 0.05% of a given value or range.

The term “activation” used in the context of human FLT3 refers to the change of human FLT3 from its inactive to its active state. In one embodiment, the change from the inactive to active state generally involves dimerization of FLT3 monomers and phosphorylation of the tyrosine-kinase domain. In another embodiment, the change does not require ligand binding (i.e., is ligand independent or constitutive). In another embodiment, the change from the inactive to active state involves ligand binding, dimerization of FLT3 monomers and phosphorylation of the tyrosine-kinase domain (i.e., is ligand dependent). In another embodiment, the change from the inactive to active state involves phosphorylation of the tyrosine-kinase domain without ligand Activation of FLT3 can be triggered, for example, by FLT3 ligand binding to FLT3 or by any of a number of FLT3-activating mutations. Activation of FLT3 can result in pFLT3.

As used herein, “administer” or “administration” refers to the act of injecting or otherwise physically delivering a substance as it exists outside the body into a patient, such as by oral, mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or a symptom thereof, is being prevented, administration of the substance typically occurs before the onset of the disease or symptoms thereof.

As used herein, an “agonist” or “activator” of FLT3 refers to a molecule that is capable of increasing or otherwise enhancing FLT3 phosphorylation or FLT3 activation.

As used herein, an “antagonist” or “inhibitor” of FLT3 refers to a molecule that is capable of inhibiting or otherwise decreasing FLT3 phosphorylation or FLT3 activation. In certain embodiments, the antagonist of FLT3 is AC220. In other certain embodiments, FLT3 antagonists comprises AC220, CEP-701, PKC-412, MLN518, sorafenib, sunitinib, KW-2449, AP-24534 and CHIR-258. In other certain embodiments, FLT3 antagonists further comprises R-406 and CGP-52421.

The term “antibody” and “immunoglobulin” or “Ig” may be used interchangeably herein. The term “antibody” refers to all types of immunoglobulins, including The antibodies of the invention can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule, or any antigen-recognition (or antigen-binding) fragments thereof. The antibodies may be monoclonal or polyclonal and may be of any species of origin, including, but not limited to, mouse, rat, rabbit, horse, or human, or may be chimeric antibodies. See, e.g., Walker et al., Molec. Immunol. 1989; 26: 403-411; Morrision et al., Proc. Nat'l. Acad. Sci. 1984; 81: 6851; Neuberger et al., Nature 1984; 312: 604. The antibodies may be recombinant monoclonal antibodies produced according to the methods disclosed in U.S. Pat. No. 4,474,893 (Reading) or U.S. Pat. No. 4,816,567 (Cabilly et al.). The antibodies may also be chemically constructed by specific antibodies made according to the method disclosed in U.S. Pat. No. 4,676,980 (Segel et al.). Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′)₂ fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., antigen binding domains or molecules that contain an antigen-binding site that immunospecifically binds to human total FLT3 or human pFLT3 (e.g., an anti-phosphotyrosine antibody). In certain embodiments, the antibody is an IgG antibody, such a monoclonal IgG antibody.

The terms “antibodies that immunospecifically bind to human total FLT3,” “antibodies that immunospecifically bind to a human FLT3 epitope,” “anti-human FLT3 antibodies” and analogous terms are used interchangeably herein and refer to antibodies and fragments thereof, that specifically bind to a human FLT3 polypeptide, such as a human FLT3 antigen or epitope, regardless of the phosphorylation levels of FLT3. An antibody or a fragment thereof that immunospecifically binds to a human FLT3 antigen may be cross-reactive with related antigens. Preferably, an antibody or a fragment thereof that immunospecifically binds to a human FLT3 antigen does not cross-react with other antigens. An antibody or a fragment thereof that immunospecifically binds to a human FLT3 antigen can be identified, for example, by immunoassays, BIAcore, or other techniques known to those of skill in the art. An antibody or a fragment thereof binds specifically to a human FLT3 antigen when it binds to a human FLT3 antigen with higher affinity than to any cross-reactive antigen as determined using experimental techniques, such as radioimmunoassays (RIAs) and ELISAs. Typically, a specific or selective reaction will be at least twice background signal or noise and more typically more than 10 times background. See, e.g., Paul, ed., 1989 Fundamental Immunology Second Edition, Raven Press, New York at pages 332-336 for a discussion regarding antibody specificity. In certain embodiments, the anti-total human FLT3 antibody is a monoclonal IgG (e.g., mouse anti-human FLT3 IgG mAb (Cat. No. MAB8121; R&D Systems, Inc.; Minneapolis, Minn.)).

The terms “antibodies that immunospecifically bind to human pFLT3,” “antibodies that immunospecifically bind to a human pFLT3 epitope,” “anti-human pFLT3 antibodies” and analogous terms are used interchangeably herein and refer to antibodies and fragments thereof, that specifically bind to only the phosphorylated form of human FLT3 polypeptide, such as a human pFLT3 antigen or epitope (e.g., an epitope comprising an phosphotyrosine). An antibody or a fragment thereof that immunospecifically binds to a human pFLT3 antigen may be cross-reactive with related antigens. In one embodiment, an antibody or a fragment thereof that immunospecifically binds to a human pFLT3 antigen does not cross-react with other antigens. An antibody or a fragment thereof that immunospecifically binds to a human pFLT3 antigen can be identified, for example, by immunoassays, BIAcore, or other techniques known to those of skill in the art. An antibody or a fragment thereof binds specifically to a human pFLT3 antigen when it binds to a human pFLT3 antigen with higher affinity than to any cross-reactive antigen as determined using experimental techniques, such as RIAs and ELISAs. Typically, a specific or selective reaction will be at least twice background signal or noise and more typically more than 10 times background. In one embodiment, the anti-pFLT3 antibody is an anti-phosphotyrosine antibody (also referred to as “anti-pTyr” or “anti-pY”) that immunospecifically binds to a one or more phosphorylated tyrosine residues of a protein or enzyme, including one or more phosphorylated tyrosine residues of human FLT3. In certain embodiments, the anti-pFLT3 antibody immunospecifically binds to one or more of phosphorylated tyrosine (Y) residues at positions 589, 591, 597, 599, 726, 842, and 955 of human pFLT3 (SEQ ID NO:1). In another embodiment, the anti-pFLT3 antibody immunospecifically binds to most or all of phosphorylated tyrosine (Y) residues at positions 589, 591, 597, 599, 726, 842, and 955 of human pFLT3 (SEQ ID NO:1). In some embodiments, the anti-pFLT3 antibody does not immunospecifically bind to phosphorylated tyrosine residues at positions 589 and/or 591 of human pFLT3 (SEQ ID NO:1).

The term “first antibody” refers to the antibody used to capture total FLT3 in the test sample. “First antibody” and “capture antibody” are used interchangeably herein.

The term “second antibody” refers to the antibody used to detect the presence of either total FLT3 or pFLT3. “Second antibody” and “detection antibody” are used interchangeably herein.

The term “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g., the complementarity determining regions (CDR)). The antigen binding region can be derived from any animal species, such as rodents (e.g., rabbit, rat, mouse, goat or hamster) and humans. Preferably, the antigen binding region will be of human origin.

As used herein, the term “composition” is intended to encompass a product containing the specified ingredients in, optionally, the specified amounts, as well as any product which results, directly or indirectly, from combination of the specified ingredients in, optionally, the specified amounts.

The term “autophosphorylation” refers to the transfer of a phosphate group by a protein kinase either to a residue in the same kinase molecule (cis) or to a residue in a different kinase molecule (trans) but of the same type.

The term “compound” as used herein includes experimental small molecules, FDA-approved small molecule therapeutics, antibodies developed for antibody-directed therapy and other “therapeutic agents” as defined herein.

The term “effective amount” as used herein refers to the amount of a therapy (e.g., a FLT3 agonist or antagonist) which is sufficient to reduce and/or ameliorate the severity and/or duration of a given disease (e.g., a FLT3-mediated disease) and/or a symptom related thereto. This term also encompasses an amount necessary for the reduction or amelioration of the advancement or progression of a given disease, reduction or amelioration of the recurrence, development or onset of a given disease, and/or to improve or enhance the prophylactic or therapeutic effect(s) of another therapy. In some embodiments, the effective amount is from about 0.1 mg/kg (mg of compound per kg weight of the subject) to about 100 mg/kg. In certain embodiments, an effective amount of a compound provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In other embodiments, the effective amount of a compound is about 135 mg, about 200 mg, about 300 mg, about 450 mg, about 675 mg or about 1000 mg (or a range therein). In certain embodiments, the therapy is AC220.

The term “epitope” as used herein refers to a localized region on the surface of an antigen, such as a human FLT3 polypeptide or a human FLT3 polypeptide fragment, that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody immunospecifically binds as determined by any method well known in the art, for example, by the immunoassays described herein. Antigenic epitopes need not necessarily be immunogenic. Epitopes usually consist of chemically active surface groupings of molecules such as amino acids or sugar side chains and have specific three dimensional structural characteristics as well as specific charge characteristics. A region of a polypeptide contributing to an epitope may be contiguous amino acids of the polypeptide or the epitope may come together from two or more non-contiguous regions of the polypeptide The epitope may or may not be a three-dimensional surface feature of the antigen. In certain embodiments, a human FLT3 epitope is a three-dimensional surface feature of a human FLT3 polypeptide (e.g., in the native form). In other embodiments, a human FLT3 epitope is linear feature of a human FLT3 polypeptide (e.g., in a native or denatures form of the human FLT3 polypeptide). Antibodies used in the methods provided herein may immunospecifically bind to an epitope of the denatured form of human FLT3, an epitope of the native form of human FLT3, or both the denatured form and the native form of human FLT3. In specific embodiments, the antibodies used in the methods provided herein immunospecifically bind to the extracellular domain of native form of human FLT3.

The term “extracellular region” or “extracellular domain” refers to the protein or polypeptide segment on or outside the cell membrane. The extracellular region or extracellular domain of human FLT3 encompasses approximately amino acid residues 27-543 at the N-terminus of FLT3 (SEQ ID NO:1).

The term “FLT3” refers to the polypeptides (“polypeptides,” “peptides” and “proteins” are used interchangeably herein) comprising the following amino acid sequence (993 amino acids):

```
(SEQ ID NO: 1)
                10 20 30 40 50 60
MPALARDGGQ LPLLVVFSAM IFGTITNQDL PVIKCVLINH KNNDSSVGKS SSYPMVSESP
                70 80 90 100 110 120
EDLGCALRPQ SSGTVYEAAA VEVDVSASIT LQVLVDAPGN ISCLWVFKHS SLNCQPHFDL
              130 140 150 160 170 180
QNRGVVSMVI LKMTETQAGE YLLFIQSEAT NYTILFTVSI RNTLLYTLRR PYFRKMENQD
              190 200 210 220 230 240
ALVCISESVP EPIVEWVLCD SQGESCKEES PAVVKKEEKV LHELFGTDIR CCARNELGRE
              250 260 270 280 290 300
CTRLFTIDLN QTPQTTLPQL FLKVGEPLWI RCKAVHVNHG FGLTWELENK ALEEGNYFEM
              310 320 330 340 350 360
STYSTNRTMI RILFAFVSSV ARNDTGYYTC SSSKHPSQSA LVTIVEKGFI NATNSSEDYE
              370 380 390 400 410 420
IDQYEEFCFS VRFKAYPQIR CTWTFSRKSF PCEQKGLDNG YSISKFCNHK HQPGEYIFHA
              430 440 450 460 470 480
ENDDAQFTKM FTLNIRRKPQ VLAEASASQA SCFSDGYPLP SWTWKKCSDK SPNCTEEITE
              490 500 510 520 530 540
GVWNRKANRK VFGQWVSSST LNMSEAIKGF LVKCCAYNSL GTSCETILLN SPGPFPFIQD
              550 560 570 580 590 600
NISFYATIGV CLLFIVVLTL LICHKYKKQF RYESQLQMVQ VTGSSDNEYF YVDFREYEYD
              610 620 630 640 650 660
LKWEFPRENL EFGKVLGSGA FGKVMNATAY GISKTGVSIQ VAVKMLKEKA DSSEREALMS
              670 680 690 700 710 720
ELKMMTQLGS HENIVNLLGA CTLSGPIYLI FEYCCYGDLL NYLRSKREKF HRTWTEIFKE
              730 740 750 760 770 780
HNFSFYPTFQ SHPNSSMPGS REVQIHPDSD QISGLHGNSF HSEDEIEYEN QKRLEEEEDL
              790 800 810 820 830 840
NVLTFEDLLC FAYQVAKGME FLEFKSCVHR DLAARNVLVT HGKVVKICDF GLARDIMSDS
              850 860 870 880 890 900
NYVVRGNARL PVKWMAPESL FEGIYTIKSD VWSYGILLWE IFSLGVNPYP GIPVDANFYK
              910 920 930 940 950 960
LIQNGFKMDQ PFYATEEIYI IMQSCWAFDS RKRPSFPNLT SFLGCQLADA EEAMYQNVDG
              970 980 990
RVSECPHTYQ NRRPFSREMD LGLLSPQAQV EDS
```

In specific embodiments, the extracellular domain spans amino acids 27-543, the transmembrane domain spans amino acids 544 to 563, and the cytoplasmic domain spans amino acids 564 to 993. In certain embodiments, the FLT3 amino acid sequence comprises one or more of the following amino acid substitutions: D to G at position 7; V to A as position 158; T to M at position 227; D to N at position 324; D to V at position 358; V to I at position 557; G to A as position 8; QL to TV at positions 10 to 11; A to R at position 78; E to G at position 346; or a T to H at position 940.

The terms “pFLT3,” “phospho-FLT3,” “phosphotyrosine FLT3,” “tyrosine-phosphorylated FLT3,” “phosphorylated FLT3” and related terms are used interchangeably herein and refer to a FLT3 having one or more phosphorylated tyrosine residues. In one embodiment, pFLT3 is the activated form of FLT3. In another embodiment, pFLT3 is the constitutively active form of FLT3.

The term “FLT3-activating mutations” of “FLT3 mutations” are used interchangeably and refer to the addition, deletion or substitution of one or more base pairs in the sequence encoding the FLT3 gene occurring at one or more positions, which results in a higher level of FLT3 activity or an unregulated level of FLT3, including FLT3 constitutive activity. Such mutations comprise FLT3 duplication mutations, such as FLT3-ITD, which typically results from the duplication and tandem insertion of a portion of the juxtamembrane region and leads to constitutive autophosphorylation of human FLT3. The number of base pair duplication varies widely. In one embodiment, the internal tandem duplication is in the range of 3-400 base pairs long. In some embodiments, the FLT3-activating mutation is a duplication of a FLT3 receptor gene (e.g., exon 11 of FLT3). These mutations render the receptor constitutively active and alter signaling through FLT3-ITD compared to wild-type FLT3 receptor. In certain embodiments, a FLT3-activating mutation in a patient results in the patient having a FLT-3 mediated disease or disorder.

The terms “FLT3-mediated disease” or “FLT3-mediated disorder” include diseases associated with or implicating abnormal FLT3 activity. Abnormal FLT3 activity may arise from (1) FLT3 overexpression through gene amplification or other means (2) mutations in FLT3 or other proteins leading to constitutive activation of FLT3 (3) overexpression of the FLT3 ligand (FLT3L) (4) FLT3 expression in cells which normally do not express FLT3 or any combination of the four. Examples of FLT3-mediated diseases or disorders include disorders resulting in part from overstimulation of FLT3, resulting in part from abnormally high amount of FLT3 activity due to abnormally high amount of FLT3L or resulting in part from mutations in FLT3. It is known that abnormal FLT3 activity has been implicated in the pathogenesis of a number of diseases, including inflammatory and autoimmune diseases, cell proliferative disorders, neoplastic disorders and cancers as described herein. In some embodiments, a FLT3-mediated disease or disorder is the result of, for example, duplications of a FLT3 receptor gene (e.g., exon 11 of FLT3). In certain embodiments, a FLT3-mediated disease is a leukemia. In another embodiment, the leukemia is ALL, AML, MDS and/or TMDS.

“FLT3 inhibitors” are compounds that have activity against the FLT3 kinase. FLT3 inhibitors include AC220, CEP-701, PKC-412, MLN518, sorafenib, sunitinib, KW-2449, AP-24534 and CHIR-258.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, human FLT3 fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a human FLT3 polypeptide or an antibody that immunospecifically binds to a human FLT3 polypeptide. In a specific embodiment, a fragment of a human FLT3 polypeptide or an antibody that immunospecifically binds to a human FLT3 antigen retains at least 1, at least 2, or at least 3 functions of the polypeptide or antibody.

The term “intracellular region” or “intracellular domain” refers to the protein or polypeptide segment inside the cell membrane. The intracellular region or domain of human FLT3 encompasses approximately amino acid residues 564 to 993 of human FLT3 (SEQ ID NO:1).

The term “in vivo,” in the context of samples, refers to tissue samples obtained from a subject, e.g., a patient, such as a human patient, including biological samples such as biological fluids, e.g., blood, plasma, serum, bone marrow, spinal fluid, brain fluid, or tissues, such as lymph tissue, a thin layer cytological sample, a fresh frozen tissue sample or a tumor tissue, including tumor tissue that develops from the implantation of foreign tumor cells (i.e. xenograft). The term “in vivo” is to be distinguished from the term “in vitro” which encompasses cells or cell lines or biomolecular components of cells, that have been cultured or propagated outside of a living organism. In vitro samples include human cell lines expressing FLT3 or pFLT3, including MV4:11, RS4:11, SEM, MOLM-1, MOLM-13, MOLM-14, REH, BV173 and EOL-1.

The term “juxtamembrane region” or “juxtamembrane domain” refers to the protein or polypeptide segment in a receptor that connects the transmembrane helix to the kinase domain. The juxtamembrane region or juxtamembrane domain of human FLT3 encompasses approximately amino acid residues 572-603 of human FLT3 (SEQ ID NO:1).

The term “kinase domain” refers to the protein or polypeptide segment that possesses the catalytic activity, which transfers the λ-phosphate from nucleotide triphosphates (often ATP) to one or more amino acid residues in a protein or polypeptide substrate side chain, resulting in a conformational change affecting protein function. The kinase domain N-lobe of human FLT3 encompasses approximately amino acid residues 604-710 of human FLT3 (SEQ ID NO:1). The kinase domain C-lobe of human FLT3 encompasses approximately amino acid residues 781-958 of human FLT3 (SEQ ID NO:1).

The term “kinase insert region” or “kinase insert domain” refers to the protein or polypeptide segment in a protein kinase through which two different kinase domains are linked (i.e., links the kinase domain N-lobe and C-lobe of FLT3). The kinase insert region or kinase insert domain of human FLT3 refers to the protein segment of human FLT3 that connects the two tyrosine kinase domains of human FLT3, and encompasses approximately amino acid residues 711-780 of human FLT3 (SEQ ID NO:1).

The term “leukemia” refers to malignant neoplasms found in hematopoietic cells of the lymphoid lineage, myeloid lineage or a mixture of the two. Leukemia, includes, but is not limited to, chronic lymphocytic leukemia, chronic myelocytic leukemia, acute lymphoblastic leukemia, acute myelogenous leukemia and acute myeloblastic leukemia. The leukemia can be relapsed, refractory, or resistant to conventional therapy.

As used herein, the terms “manage,” “managing,” and “management” refer to the beneficial effects that a patient derives from a therapy (e.g., a prophylactic or therapeutic agent), which does not result in a cure of the disease or condition. In certain embodiments, a subject is administered one or more therapies (e.g., prophylactic or therapeutic agents, such as an agonist or antagonist of FLT3 phosphorylation) to “manage” a FLT3-mediated disease or disorder (e.g., a leukemia, such as ALL or AML), or one or more symptoms thereof, so as to prevent the progression or worsening of the disease.

The term “monoclonal antibody” refers to an antibody obtained from a population of homogenous or substantially homogeneous antibodies, and each monoclonal antibody will typically recognize a single epitope on the antigen. In certain embodiments, a “monoclonal antibody,” as used herein, is an antibody produced by a single hybridoma or other cell, wherein the antibody immunospecifically binds to only a human FLT3 epitope as determined, e.g., by ELISA or other antigen-binding or competitive binding assay known in the art. The term “monoclonal” is not limited to any particular method for making the antibody. For example, monoclonal antibodies used in the methods provided herein may be made by the hybridoma method as described in Kohler et al.; Nature, 256:495 (1975) or may be isolated from phage libraries using the techniques known in the art. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York).

The term “MSD system” used herein refers to methods incorporating detection and quantification by the MSD ECL detection system (Meso Scale Discovery, Gaithersburg, Md.). Such system employs, for example, a ruthenium (II) tri-bipyridine-(4-methylsulfonate) NHS ester label that emits light upon electrochemical activation (e.g., SULFO-TAG™, Meso Scale Discovery (Gaithersburg, Md.)). ECL measurements can be carried out using screen-printed carbon ink electrodes patterned on the bottom of specially designed multi-well or multi-domain multi-well plates, for example, as described below and in U.S. Pat. No. 7,063,946, which is herein incorporated by reference in its entirety (e.g., MULTI-ARRAY® and MULTI-SPOT® microplates, MSD (Gaithersburg, Md.)). Each well of the plate can have a patterned working electrode comprising 1, 4, 7, or 10 assay domains (approximately in the center of the well) that are exposed regions of electrode surface, which are defined, for example, by a patterned dielectric layer. The dielectric layer can be used to confine small volumes of liquid to specific assay domains. Each well can also have two counter electrodes surfaces (e.g., approximately at two edges of the well). ECL from the ECL labels on the surface of the carbon electrodes can be induced and measured using an imaging plate reader compatible with the MSD system (e.g., SECTOR® Imager 6000 and SECTOR® Imager 2400, MSD (Gaithersburg, Md.)).

The term “naturally occurring” or “native” when used in connection with biological materials such as nucleic acid molecules, polypeptides, host cells, and the like, refers to those which are found in nature and not manipulated by a human being. The term “native form” used herein in the context of human FLT3 refers to the most typical three-dimensional conformation of human FLT3 in its biological environment that determines its function without undergoing or having undergone structural changes by external forces such as denaturing agents.

“Polyclonal antibodies” as used herein refers to an antibody population generated in an immunogenic response to a protein having many epitopes and thus includes a variety of different antibodies directed to the same and to different epitopes within the protein. Methods for producing polyclonal antibodies are known in the art (See, e.g., see, for example, Chapter 11 in: Short Protocols in Molecular Biology, (2002) 5th Ed., Ausubel et al., eds., John Wiley and Sons, New York).

The term “phosphorylation” refers to the introduction of a phosphoryl group into a molecule through the formation of a ester bond between the molecule and the phosphoric acid.

As used herein, the terms “prevent,” “preventing,” and “prevention” refer to the total or partial inhibition of the development, recurrence, onset or spread of a FLT-mediated disease and/or symptom related thereto, resulting from the administration of a therapy or combination of therapies provided herein (e.g., a combination of prophylactic or therapeutic agents).

As used herein, the term “prophylactic agent” refers to any agent that can totally or partially inhibit the development, recurrence, onset or spread of a FLT3-mediated disease (e.g., a leukemia, such as ALL or AML) and/or symptom related thereto in a subject. In certain embodiments, the term “prophylactic agent” refers to an antagonist of FLT3 phosphorylation, such as AC220. Preferably, a prophylactic agent is an agent which is known to be useful to or has been or is currently being used to prevent a FLT3-mediated disease and/or a symptom related thereto or impede the onset, development, progression and/or severity of a FLT3-mediated disease and/or a symptom related thereto.

The term “receptor tyrosine kinase” refers to a cell-surface receptor that has an intracellular protein tyrosine kinase domain, which, in its active state, transfers the λ-phosphate of nucleotide triphosphates (often ATP) to tyrosine residues in its polypeptide or protein substrates.

The term “relapsed” refers to a situation where a subject or a mammal, which has had a remission of cancer after therapy has a return of cancer cells.

As used herein, the terms “subject” and “patient” are used interchangeably. As used herein, a subject is preferably a mammal such as a non-primate (e.g., cows, pigs, horses, cats, dogs, rats, etc.) or a primate (e.g., monkey and human), most preferably a human. In one embodiment, the subject is a mammal, preferably a human, having a FLT3-mediated disease (e.g., a leukemia, such as ALL or AML). In another embodiment, the subject is a mammal, preferably a human, at risk of developing a FLT3-mediated disease.

As used herein, the term “tag” or “label” are used interchangeably and refer to any type of moiety that is attached to an antibody or antigen binding fragment thereof, or other polypeptide used in the methods provided herein, such as an anti-FLT3 antibody, and anti-pFLT3 antibody, an anti-phosphotyrosine antibody, and/or streptavidin. In certain embodiments, the label is a ruthenium (II) tri-bipyridine-(4-methylsulfonate) NHS ester. The term “detectable” or “detection” with reference to an antibody or tag refers to any antibody or tag that is capable of being visualized or wherein the presence of the antibody or tag is otherwise able to be determined and/or measured (e.g., by quantitation). Non-limiting examples of a detectable tag include fluorescent or other chemiluminescent tags, and tags that can be amplified and quantitated using PCR. In certain embodiments, the secondary antibody used in the methods provided herein is a biotinylated secondary antibody that is used in combination with a labeled streptavidin, such as streptavidin labeled with a ruthenium (II) tri-bipyridine-(4-methylsulfonate) NHS ester.

As used herein, the term “therapeutic agent” refers to any agent that can be used in the treatment, management or amelioration of a FLT3-mediated disease (e.g., a leukemia, such as ALL or AML) and/or a symptom related thereto. In certain embodiments, the term “therapeutic agent” refers to an antibody of the invention. In certain other embodiments, the term “therapeutic agent” refers to an antagonist of FLT3 phosphorylation, such as AC220. Preferably, a therapeutic agent is an agent which is known to be useful for, or has been or is currently being used for the treatment, management or amelioration of a FLT3-mediated disease or one or more symptoms related thereto.

The combination of therapies (e.g., use of prophylactic or therapeutic agents) which is more effective than the additive effects of any two or more single therapy. For example, a synergistic effect of a combination of prophylactic and/or therapeutic agents permits the use of lower dosages of one or more of the agents and/or less frequent administration of said agents to a subject with a FLT3-mediated disease (e.g., a leukemia, such as ALL or AML). The ability to utilize lower dosages of prophylactic or therapeutic therapies and/or to administer said therapies less frequently reduces the toxicity associated with the administration of said therapies to a subject without reducing the efficacy of said therapies in the prevention, management, treatment or amelioration of a FLT3-mediated disease. In addition, a synergistic effect can result in improved efficacy of therapies in the prevention, or in the management, treatment or amelioration of a FLT3-mediated disease. Finally, synergistic effect of a combination of therapies (e.g., prophylactic or therapeutic agents) may avoid or reduce adverse or unwanted side effects associated with the use of any single therapy.

As used herein, the term “therapy” refers to any protocol, method and/or agent that can be used in the prevention, management, treatment and/or amelioration of a FLT3-mediated disease (e.g., a leukemia, such as ALL or AML). In certain embodiments, the terms “therapies” and “therapy” refer to a biological therapy, supportive therapy, and/or other therapies useful in the prevention, management, treatment and/or amelioration of a FLT3-mediated disease known to one of skill in the art such as medical personnel.

As used herein, the terms “treat,” “treatment” and “treating” refer to the reduction or amelioration of the progression, severity, and/or duration of a FLT3-mediated disease (e.g., e.g., a leukemia, such as ALL or AML) resulting from the administration of one or more therapies, including, but not limited to, the administration of one or more prophylactic or therapeutic agents, such as an antagonist of FLT3 phosphorylation, such as AC220.

### BRIEF DESCRIPTION OF THE DRAWINGS

FIG. 1 shows the type of results obtained by an immunoprecipitation/Western immunoblot (IP/Western blot) assay for Patient C. An IP/Western blot was performed to detect levels of pFLT3 and total FLT3 in blood samples from patients receiving AC220 therapy.

FIGS. 2A-2C depict the amount of pFLT3 present in Patient A over the course of a 24 hour period (data not shown for 4, 6 and 9 hour timepoints) following treatment with AC220. pFLT3 levels in in vivo samples were determined using methods of the invention. (A) The trend in the ratio of pFLT3 to total FLT3 in blood samples drawn from Patient A over the course of 24 hours with the initial ratio set at 100%. (B) Measure of pFLT3 levels using the in vivo MSD ELISA readout (C) Measure of total FLT3 levels using the in vivo MSD ELISA readout.

FIGS. 3A-3C depict the amount of pFLT3 present in Patient B over the course of a 24 hour period following treatment with AC220. pFLT3 levels in in vivo samples were determined using methods of the invention. (A) The trend in the ratio of pFLT3 to total FLT3 in blood samples drawn from Patient B at Day 1, Day 8 and Day 15 of a treatment cycle with AC220, with the initial ratio set at 100%. (B) Measure of pFLT3 levels using the in vivo MSD ELISA readout (C) Measure of total FLT3 levels using the in vivo MSD ELISA readout.

FIGS. 4A-4C depict the amount of pFLT3 present in Patient C over the course of a 24 hour period (data not shown for 4, 6 and 9 hour timepoints) following treatment with AC220. pFLT3 levels in in vivo samples were determined using methods of the invention. (A) The trend in the ratio of pFLT3 to total FLT3 in blood samples drawn from Patient C at prebleed (before dose) and at 2 hours and 24 hours post dose. (B) Measure of pFLT3 levels using the in vivo MSD ELISA readout (C) Measure of total FLT3 levels using the in vivo MSD ELISA readout.

FIG. 5 depict the amount of pFLT3 present in Patient D over the course of a 24 hour period (data not shown for 4, 6 and 9 hour timepoints) following treatment with AC220. pFLT3 levels in in vivo samples were determined using methods of the invention. (A) The trend in the ratio of pFLT3 to total FLT3 in blood samples drawn from Patient C at prebleed (before dose) and at 2 hours and 24 hours post dose. (B) Measure of pFLT3 levels using the in vivo MSD ELISA readout (C) Measure of total FLT3 levels using the in vivo MSD ELISA readout.

FIG. 6 depicts the correlation between absolute blast count and pFLT3 or total FLT3 levels. “Pb” refers to peripheral blood. (A) Correlation between absolute blast count (in thousands per microliter (1000/μL)) and total FLT3 levels among 45 clinical patient undergoing AC220 treatment. (B) Correlation between absolute blast count (in thousands per microliter (1000/μL) and pFLT3 levels among 45 clinical patient undergoing AC220 treatment. pFLT3 levels in in vivo samples were determined using methods of the invention.

FIG. 7 depicts the correlation between pFLT3 inhibition and percent peripheral blast reduction.

FIGS. 8A-8B depict the correlation between FLT3-ITD genotyping status and total FLT3 expression. (A) Correlation among 34 clinical patient undergoing AC220 treatment. (B) Correlation between FLT3-ITD genotyping status and total (tFLT3) expression among 34 clinical patient undergoing AC220 treatment. pFLT3 levels in in vivo samples were determined using methods of the invention.

FIG. 9 depicts the levels of pFLT3 and total FLT3 measured in tumor samples extracted from a MV4:11 mouse tumor xenograft experiment in which the mice received 10 mg/kg PO AC220. (A) pFLT3 and (B) FLT3 levels, respectively, in four untreated mice tested in triplicate. (C) pFLT3 and (D) FLT3 levels, respectively, in four treated mice at the 2 hour post dose timepoint, tested in triplicate. (E) pFLT3 (F) FLT3 levels, respectively, in treated mice at the 24 hour post dose timepoint, tested in triplicate. pFLT3 levels in in vivo samples were determined using methods of the invention.

FIG. 10A-10C depicts the levels of pFLT3 present in Patient E over the course of a 24 hour period (data not shown for 4, 6 and 9 hour timepoints) following treatment with AC220. (A) The trend in the ratio of pFLT3 to total FLT3 in blood samples drawn from Patient C at prebleed (before dose) and at 2 hours and 24 hours post dose. (B) Measure of pFLT3 levels using the in vivo MSD ELISA readout (C) Measure of total FLT3 levels using the in vivo MSD ELISA readout.

FIG. 11A-B depict the comparison of MSD ELISA signals (y-axis) from two different total Flt 3 capture abs (R&D MAB8121 vs. Santa Cruz SC479).

FIG. 12A-B depict the comparison of MSD ELISA signals (y-axis) from two different total detection antibodies (R&D BAF812 vs. Santa Cruz SC479) with R&D total capture antibody MAB8121 for FLT3.

FIG. 13A-D depict the titration of MV4:11 cells in blood—sensitivity of IP/Western (A, B) vs. MSD ELISA (C, D) for detecting total and phospho FLT3 from normal blood.

FIGS. 14 A and B depict the pFLT3 and tFLT3 IP Western blot of normal blood samples titrated with different amounts of pFLT3-generating MV4:11 cells. FIGS. 14 C and D depict the quantitation of the tFLT3 and pFLT3 IP Western blot signal using the Odyssey Infrared Imaging System.

FIG. 15 depicts the inhibition of pFLT3 signal by AC220 using SC479 to capture total FLT3.

FIG. 16 depicts the inhibition of pFLT3 signal by AC220 using R&D antibody (MAB8121) to capture total FLT3.

FIG. 17A-B depict the comparison of dynamic range of DMSO/1 μM AC220 pTyr inhibition using SC479 vs. R&D capture antibody (MAB8121).

FIG. 18 depicts the IC50s generated for a select number of FLT3 inhibitors, generated from a pFLT3 MSD ELISA assay.

FIG. 19 depicts the IC50s generated for FLT3 inhibitors, generated from the pFLT3 MSD ELISA assay using either MV4:11 (ITD) cells as the source of constitutively active pFLT3 or RS4:11 as the source of ligand-activated wild-type pFLT3.

FIG. 20 depicts the percentage of patients belonging to each cohort in the AC220 phase I study (where the x-axis shows the dose of AC220 at each cohort) who achieved greater than 25% (top graph), greater than 50% (center graph) or greater the 75% reduction (bottom graph) in pFLT3 levels. pFLT3 levels in in vivo samples were determined using methods of the invention.

### DETAILED DESCRIPTION

Provided herein are methods for detecting the presence of human pFLT3 and total FLT3 in samples, e,g., in vivo samples such as patient blood or tissue samples. An exemplary assay provided herein is an enzyme-linked immunosorbent assay (ELISA), for example, a sandwich ELISA. Also provided herein are methods for detecting FLT3 phosphorylation in a sample. Further provided are methods for diagnosing a patient having a FLT3-activating mutation. Also provided are methods for identifying a test compound that is an agonist or antagonist of human FLT3 phosphorylation. Further provided are methods for determining the efficacy of a test compound for increasing, decreasing or otherwise modulating human FLT3 phosphorylation in a patient. Also provided are kits for carrying out said methods.

Certain methods described herein may be applied to examining an in vivo sample for the presence of human pFLT3, in which phosphorylation of FLT3 at one or more of tyrosine residues of the FLT3 (e.g., 589, 591, 597, 599, 726, 842 and 955) has diagnostic and/or prognostic value to predict the outcome of disease or the response to the treatment.

Certain other methods provided herein have broad applications to be used clinically, either in diagnoses or prognoses. Such applications include, but not limited to, detecting FLT3 phosphorylation in an in vivo sample, diagnosing a patient having a FLT3-activating mutation, e.g., FLT3-ITD mutation, as a therapeutic aid for patient treatment selection and monitoring the status of a FLT3-mediated disease or disorder, or a symptom thereof, in a patient, e.g.,. a patient having a leukemia, such as ALL or AML.

Thus, in one aspect, provided herein is a method for detecting the presence of pFLT3 in a sample, e.g., an in vivo sample (such as blood, blood lysate, plasma, spinal fluid, cerebral fluid or bone marrow aspirate), said method comprising (a) contacting the sample with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3 or a phosphotyrosine epitope, and wherein the second antibody immunospecifically binds to a different FLT3 epitope than the first antibody; (d) removing unbound second antibody; and (e) detecting the presence of second antibody bound to the sample; wherein an increase in the amount of second antibody bound to the sample, as compared to a control sample having no pFLT3, indicates the presence of human pFLT3 in the sample. In certain embodiments, the second antibody is a biotinylated anti-phosphortyrosine antibody, and the method further comprises contacting the second antibody with a labeled streptavidin, wherein the labeled streptavidin is optionally detected by ECL. In certain embodiments, the method further comprises providing results from the assay to personnel, e.g., at a medical facility, such as a doctor, nurse or other medical professional. In other embodiments, the method further comprises providing therapeutic options to personnel, e.g., at a medical facility, such as a doctor, nurse or other medical professional.

In certain embodiments, the method further comprises detecting total FLT3 in the sample (such as blood, blood lysate, plasma, spinal fluid, cerebral fluid or bone marrow aspirate). For example, in certain embodiments, a sample can be split into two portions, in which the first portion of the sample is assayed for the presence of pFLT3, such as described above, and the second portion of the sample is assayed for the presence of total FLT3 by substituting a detectable anti-pFLT3 antibody in step (c) for a detectable anti-total FLT3 secondary antibody, wherein the anti-total FLT3 detectable secondary antibody immunospecifically binds to a different FLT3 epitope than the first antibody. In one embodiment, the anti-total FLT3 secondary antibody is an anti-total FLT3 polyclonal antibody of a species other than human, such as rabbit or goat. In some embodiments, the anti-total FLT3 secondary antibody is biotinylated, which is then contacted with a labeled streptavidin. In these embodiments, the percentage of pFLT3 present of the total FLT can be calculated as follows: (amount of pFLT3/amount of total FLT3)×100%.

In a second aspect, provided herein are methods for detecting FLT3 phosphorylation in a sample, e.g., an in vivo sample (such as blood, blood lysate, plasma, spinal fluid, cerebral fluid or bone marrow aspirate), said method comprising: (a) contacting the sample with an immobilized first antibody that immunospecifically binds to human total FLT3; (b) removing unbound sample; (c) contacting the sample bound to the immobilized first antibody with a detectable second antibody, wherein the second antibody immunospecifically binds to only the phosphorylated form of human FLT3 or a phosphotyrosine epitope, and wherein the second antibody immunospecifically binds to a different FLT3 epitope than the first antibody; (d) removing unbound second antibody; and (e) detecting the presence of second antibody bound to the sample; wherein an increase in the amount of second antibody bound to the sample, as compared to a control sample having no pFLT3, indicates FLT3 phosphorylation in the sample.

In certain embodiments, the second antibody is a biotinylated anti-phosphortyrosine antibody, and the method further comprises contacting the second antibody with a labeled streptavidin, wherein the labeled streptavidin is optionally detected by ECL. In certain embodiments, the method further comprises providing results from the assay to personnel, e.g., at a medical facility, such as a doctor, nurse or other medical professional. In other embodiments, the method further comprises providing therapeutic options to personnel, e.g., at a medical facility, such as a doctor, nurse or other medical professional.

Yet other methods provided herein can be used for drug discovery to identify compounds that are useful to modulate FLT3 activity. Such methods include, but not limited to, identifying a test compound that is an agonist of human FLT3 phosphorylation, identifying a test compound that is an antagonist of human FLT3 phosphorylation, and determining the efficacy of a test compound for increasing, decreasing or otherwise modulating human FLT3 phosphorylation in a patient. In yet another embodiment, provided herein are methods for simultaneously detecting pFLT3 and tFLT3 present in a given sample, said method comprising: (a) contacting the sample with a first antibody immobilized in a first and second well of a multiwell plate, that immunospecifically binds to human FLT3; (b) removing unbound sample (c) contacting the sample contained in the first well with a detectable anti-FLT3 antibody that immunospecifically binds to a human FLT3 epitope wherein the anti-FLT3 antibody binds to a different FLT3 epitope than the first antibody; (d) contacting the sample contained in the second well with a detectable anti-pFLT3 antibody that immunospecifically binds to a phosphorylated form of FLT3 or a phosphotyrosine eptiope wherein the detectable anti-pFLT3 antibody immunospecifically binds to a different epitope than the first antibody; and (e) simultaneously detecting or otherwise measuring the presence of the detectable antibody in the first and second wells. In yet another embodiment, the method further comprises reporting the pFLT3 signal detected as a ratio of pFLT3 signal to tFLT3 signal. In one embodiment, the first antibody is an antibody that immunospecifically binds to an epitope present in the extracellular domain of human FLT3. In another embodiment, the anti-pFLT3 antibody immunospecifically binds to a phosphotyrosine epitope.

In a third aspect, provided herein are methods for diagnosing a patient having a FLT3-activating mutation, said method comprising: (a) contacting a sample from the patient with an immobilized first antibody that immunospecifically binds to human total FLT3; (b) removing unbound sample; (c) contacting the sample bound to the immobilized first antibody with a detectable second antibody, wherein the second antibody immunospecifically binds to aphosphorylated form of human FLT3 or a phosphotyrosine epitope, and wherein the second antibody immunospecifically binds to a different FLT3 epitope than the first antibody; (d) removing unbound second antibody; and (e) detecting or otherwise measuring the presence of second antibody bound to the sample. In certain embodiments, the presence of second antibody bound to the sample, as compared to a second sample lacking pFLT3, correlates with the presence of the FLT3-activating mutation in the patient. In some embodiments, the method further comprises (f) comparing the amount of second antibody detected to a probability table and assigning a probability of the patient having a FLT3-activating mutation to the amount of detected second antibody, whereby the patient is diagnosed with or not diagnosed with the FLT3-activating mutation (see, e.g., Example 8). In certain embodiments, the method further comprises providing results from the assay to personnel, e.g., at a medical facility, such as a doctor, nurse or other medical professional. In other embodiments, the method further comprises providing therapeutic options to personnel, e.g., at a medical facility, such as a doctor, nurse or other medical professional. In one embodiment, the patent has a FLT3-mediated disease or disorder, or a symptom thereof. In other embodiments, the FLT3-mediated disease or disorder is a leukemia, such as AML or ACL. In yet other embodiments, the patient has a FLT3 mediated disease or disorder resulting from a FLT3-activating mutation, such as. a FLT3-ITD mutation. In certain embodiments, the second antibody is a biotinylated anti-phosphortyrosine antibody, and the method further comprises contacting the second antibody with a labeled streptavidin, wherein the labeled streptavidin is optionally detected by ECL.

In certain embodiments, provided herein is a method of classifying a patient for eligibility for FLT3 therapy with a compound or other therapeutic agent, such as a small molecule inhibitor comprising:(a) providing a sample from a patient; (b) determining amount of pFLT3 in the sample (e.g., using the methods provided herein); and (c) classifying the patient as eligible to receive the FLT3 therapy based on the amount of pFLT3 in the sample. In specific embodiments, the determining step (b) is performed using an ELISA method provided herein. In one embodiment, the compound is a FLT3 small molecule inhibitor, such as AC220.

In a fourth aspect, provided herein are methods for identifying a test compound that activates human FLT3 phosphorylation or is otherwise an FLT3 agonist, said method comprising: (a) contacting a sample (such as blood, blood lysate, plasma, spinal fluid, cerebral fluid or bone marrow aspirate) comprising human FLT3 in the presence and absence of test compound; (b) contacting the sample (e.g., after a period of time sufficient to allow for the compound to contact the FLT3) with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein an increase in the amount of second antibody bound to the sample in the presence of the test compound, as compared to the amount of second antibody bound to the sample in the absence of the test compound, indicates the test compound is activates of human FLT3 phosphorylation. In certain embodiments, the second antibody is a biotinylated anti-phosphortyrosine antibody, and the method further comprises contacting the second antibody with a labeled streptavidin, wherein the labeled streptavidin is optionally detected by ECL. In some embodiments, the test compound is one of a plurality of test compounds, wherein at lest two of the test compounds differ from one another. In some embodiments, the plurality of test compounds comprises between 1 and 100,000 test compounds, between 1 and 35,000 test compounds, between 1 and 10,000 test compounds, between 1 and 1000 test compounds, between 1 and 100 test compounds, or between 1 and 10 test compounds.

In a fifth aspect, provided herein are methods for identifying a test compound that inhibits human FLT3 phosphorylation or is otherwise a FLT3 antagonist, said method comprising: (a) contacting a sample (such as blood, blood lysate, plasma, spinal fluid, cerebral fluid or bone marrow aspirate) comprising human FLT3 in the presence and absence of test compound; (b) contacting the sample (e.g., after a period of time sufficient to allow for the compound to contact the FLT3) with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein an decrease in the amount of second antibody bound to the sample in the presence of the test compound, as compared to the amount of second antibody bound to the sample in the absence of the test compound, indicates the test compound inhibits human FLT3 phosphorylation. In certain embodiments, the second antibody is a biotinylated anti-phosphortyrosine antibody, and the method further comprises contacting the second antibody with a labeled streptavidin, wherein the labeled streptavidin is optionally detected by ECL. In some embodiments, the test compound is one of a plurality of test compounds, wherein at lest two of the test compounds differ from one another. In some embodiments, the plurality of test compounds comprises between 1 and 100,000 test compounds, between 1 and 35,000 test compounds, between 1 and 10,000 test compounds, between 1 and 1000 test compounds, between 1 and 100 test compounds, or between 1 and 10 test compounds.

Certain other methods provided herein may be used to observe or otherwise monitor how a patient with a FLT3-mediated disease, such as AML, is responding to a therapy. Such information can be used, for example, to make better decisions about the optimal methods, doses, or treatments for the patient. For example, these methods are applicable where a subject has been previously diagnosed as having AML, and possibly has undergone treatment for the disease, and the methods provided herein are employed to monitor the progression of FLT3 phosphorylation or the treatment thereof. In addition, the information obtained by said methods may be used for selecting a patient suitable for FLT3 inhibitor therapy. In certain embodiments, the methods herein are use in conjunction with treatment of a patient having or suspected of having a FLT3-mediated disease or symptom thereof (e.g., a leukemia, such as AML or ALL), including a FLT3-mediated disease that is the result of the patient having a FLT3-activating mutation (e.g., FLT3-ITD). That is, in certain embodiments, the assay methods provided herein are used to monitor or otherwise track pFLT3 levels in a patient that has been (or will be) administered a FLT3 therapeutic agent, such as a FLT3 inhibitor (e.g., AC220). The assay methods provided herein can be used to track or otherwise monitor a patient's pFLT3 status in a time efficient, cost-effective manner.

Thus, in a sixth aspect, provided herein are methods for determining or monitoring the efficacy of a compound for inhibiting or decreasing human FLT3 phosphorylation in a patient, said method comprising: (a) administering the compound to the patient; (b) contacting a sample (e.g., after a period of time sufficient to allow for the compound to contact the FLT3) from the patient with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein a decrease in the amount of second antibody bound to the sample, as compared to a second sample from the patient from a different (earlier or later) time point, such as prior to administration of the compound, indicates the efficacy of the compound for decreasing human FLT3 phosphorylation in the patient. In one embodiment, the test sample is run simultaneously or in parallel with the second sample. In other embodiments, the samples are run sequentially at different times. In one embodiment, the human FLT3 phosphorylation is the result of a FLT3-activating mutation, such as a FLT3-ITD mutation. In certain embodiments, the sample is blood from a human patient, lysate of human blood or bone marrow from a patient. In certain embodiments, the compound is a FLT3 inhibitor, such as AC220, CEP-701, PKC-412, MLN518, sorafenib, sunitinib, KW-2449, AP-24534 or CHIR-258.

In certain embodiments, the method further comprises providing results from the assay to personnel, e.g., at a medical facility, such as a doctor, nurse or other medical professional. In other embodiments, the method further comprises providing therapeutic options to personnel, e.g., at a medical facility, such as a doctor, nurse or other medical professional. In one embodiment, the patent has a FLT3-mediated disease or disorder, or a symptom thereof. In other embodiments, the FLT3-mediated disease or disorder is a leukemia, such as AML or ACL. In yet other embodiments, the patient has a FLT3 mediated disease or disorder resulting from a FLT3-activating mutation, such as. a FLT3-ITD mutation. In certain embodiments, the compound is an antagonist of FLT3 phosphorylation, e.g., AC220. In certain embodiments, the second antibody is a biotinylated anti-phosphortyrosine antibody, and the method further comprises contacting the second antibody with a labeled streptavidin, wherein the labeled streptavidin is optionally detected by ECL. In some embodiments, the method is used to monitor or otherwise track a patient's responsiveness to a given FLT3 therapy by measuring pFLT3 levels in a sample (such as blood, blood lysate, plasma, spinal fluid, cerebral fluid or bone marrow aspirate) from the patient over a period of time, such as before during and/or after the treatment with the therapy over the course of a 1 hour, 2 hours, 3 hours, 4 hours, 6 hours, 8 hours, 10 hours, 12 hours, 18 hours, 24 hour, 2 day, 3 day, 4 day, 5 day, 6 day, 7 day, 2 week, 3 week, 4 week, 2 month, 3 month, 4 month, 5 month, 6 month, 7 month, 8 month, 9 month, 10, month 11 month, 1 year or more period of time.

In a seventh aspect, provided herein are methods of preventing, treating, or otherwise managing a FLT3-mediated disease or symptom thereof, said method comprising: (a) administering a compound or other therapy to the patient; (b) contacting a sample (such as blood, blood lysate, plasma, spinal fluid, cerebral fluid or bone marrow aspirate) from the patient (e.g., after a period of time sufficient to allow for the compound to contact the FLT3) with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein a decrease in the amount of second antibody bound to the sample, as compared to a sample from the patient prior to administration of the compound, indicates the compound prevents, treats, or otherwise manages human FLT3 phosphorylation in the patient. In certain embodiments, the method further comprises providing results from the assay to personnel, e.g., at a medical facility, such as a doctor, nurse or other medical professional. In other embodiments, the method further comprises providing therapeutic options to personnel, e.g., at a medical facility, such as a doctor, nurse or other medical professional. In one embodiment, the patent has a FLT3-mediated disease or disorder, or a symptom thereof. In other embodiments, the FLT3-mediated disease or disorder is a leukemia, such as AML or ACL. In yet other embodiments, the patient has a FLT3 mediated disease or disorder resulting from a FLT3-activating mutation, such as a FLT3-ITD mutation. In certain embodiments, the compound inhibits or is otherwise an antagonist of FLT3 phosphorylation. In certain embodiments, the compound is a FLT3 inhibitor, such as AC220, CEP-701, PKC-412, MLN518, sorafenib, sunitinib, KW-2449, AP-24534 or CHIR-258.

In certain embodiments, the patient is receiving an anti-cancer therapy in addition to the compound. In one embodiment, the anti-cancer therapy is an antimetabolite. In another embodiment, the anti-cancer therapy is selected from the group of anti-metabolites consisting of fludarabine, clofaribine, cytosine arabinoside, ara-c and 5-azacytidine. In certain embodiments, the second antibody is a biotinylated anti-phosphortyrosine antibody, and the method further comprises contacting the second antibody with a labeled streptavidin, wherein the labeled streptavidin is optionally detected by ECL. In some embodiments, the method is used to monitor or otherwise track a patient's responsiveness to a given FLT3 therapy by measuring pFLT3 levels in a sample (such as blood, blood lysate, plasma, spinal fluid, cerebral fluid or bone marrow aspirate) from the patient over a period of time, such as before during and/or after the treatment with the therapy over the course of a 24 hour, 2 day, 3 day, 4 day, 5 day, 6 day, 7 day, 2 week, 3 week, 4 week, 2 month, 3 month, 4 month, 5 month, 6 month, 7 month, 8 month, 9 month, 10, month 11 month, 1 year or more period of time. In some embodiments, the treatment is continued if the pFLT3 levels in the samples following treatment are lower than the pFLT3 levels in the sample prior to the start of treatment. Alternatively, in other embodiments, the treatment is ceased if the pFLT3 levels in the samples following treatment are higher than the pFLT3 levels in the sample prior to start of treatment. In another embodiment, the pFLT3 level measured in a patient sample, such as blood or plasma, is used to determine the dose of drug to be given to the patient at the beginning of treatment or to adjust the dose of the drug during the course of treatment.

In one embodiment, provided herein are methods of preventing and/or managing a FLT3-mediated disease or symptom thereof, said method comprising: (a) administering a compound to the patient; (b) contacting a sample from the patient (e.g., after a period of time sufficient to allow for the compound to contact the FLT3) with an immobilized first antibody that immunospecifically binds to human total FLT3; (c) removing unbound sample; (d) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (e) removing unbound second antibody; and (f) detecting the presence of second antibody bound to the sample; wherein a decrease in the amount of second antibody bound to the sample; and (g) comparing the amount of second antibody detected in a sample taken from the patient prior to step (a) or step (b), whereby the patient continues treatment if there is a decrease in the amount of second antibody detected in step (f) and whereby the patient ceases treatment if there is an increase in the amount of antibody detected in step (f). In certain embodiments, the method further comprises providing results from the assay to personnel, e.g., at a medical facility, such as a doctor, nurse or other medical professional. In other embodiments, the method further comprises providing therapeutic options to personnel, e.g., at a medical facility, such as a doctor, nurse or other medical professional. In one embodiment, the patent has a FLT3-mediated disease or disorder, or a symptom thereof. In other embodiments, the FLT3-mediated disease or disorder is a leukemia, such as AML or ACL. In yet other embodiments, the patient has a FLT3 mediated disease or disorder resulting from a FLT3-activating mutation, such as. a FLT3-ITD mutation. In certain embodiments, the compound inhibits or is otherwise an antagonist of FLT3 phosphorylation, e.g., AC220. In certain embodiments, the patient is receiving an anti-cancer therapy in addition to the compound. In one embodiment, the anti-cancer therapy is an antimetabolite. In another embodiment, the anti-cancer therapy is selected from the group of anti-metabolites consisting of fludarabine, clofaribine, cytosine arabinoside, ara-c and 5-azacytidine. In certain embodiments, the second antibody is a biotinylated anti-phosphortyrosine antibody, and the method further comprises contacting the second antibody with a labeled streptavidin, wherein the labeled streptavidin is optionally detected by ECL. In some embodiments, the method is used to monitor or otherwise track a patient's responsiveness to a given FLT3 therapy by measuring pFLT3 levels in a sample (such as blood, blood lysate, plasma, spinal fluid, cerebral fluid or bone marrow aspirate) from the patient over a period of time, such as before during and/or after the treatment with the therapy over the course of a 24 hour, 2 day, 3 day, 4 day, 5 day, 6 day, 7 day, 2 week, 3 week, 4 week, 2 month, 3 month, 4 month, 5 month, 6 month, 7 month, 8 month, 9 month, 10, month 11 month, 1 year or more period of time. In some embodiments, the treatment is continued if the pFLT3 levels in the samples following treatment are lower than the pFLT3 levels in the sample prior to the start of treatment. Alternatively, in other embodiments, the treatment is ceased if the pFLT3 levels in the samples following treatment are higher than the pFLT3 levels in the sample prior to start of treatment. In another embodiment, the pFLT3 level measured in a patient sample, such as blood or plasma, is used to determine the dose of drug to be given to the patient at the beginning of treatment or to adjust the dose of the drug during the course of treatment. In certain embodiments, the compound is a FLT3 inhibitor, such as AC220, CEP-701, PKC-412, MLN518, sorafenib, sunitinib, KW-2449, AP-24534 or CHIR-258.

In an eighth aspect, provided herein are methods for diagnosing a patient having a FLT3-activating mutation, e.g., a FLT3-ITD mutation, comprising detecting the amount of pFLT3 present in an in vivo sample of the patient (such as blood, blood lysate or bone marrow), and identifying the amount of pFLT3 present as corresponding to an amount of pFLT3 present in a sample having a FLT3-activating mutation, e.g., a FLT3-ITD mutation. In another embodiment, provided herein is a method for diagnosing a patient having a FLT3-mediated disease comprising quantifying the amount of pFLT3 present in an in vivo sample of the patient (such as blood, blood lysate or bone marrow). In certain embodiments, the determination is performed using a sample directly from the patient, that is, without any sample processing prior to the method being performed. As another embodiment, provided herein are methods for diagnosing a FLT3-mediated hematological malignancy, said method comprising: (a) contacting a sample (such as blood, blood lysate, plasma or bone marrow) from the patient with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (d) removing unbound second antibody; and (e) detecting the presence of second antibody bound to the sample In an eighth aspect, provided herein are methods for diagnosing a FLT3-mediated hematological malignancy, said method comprising: (a) contacting a sample (such as blood, blood lysate, plasma or bone marrow) from the patient with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (d) removing unbound second antibody; and (e) detecting the presence of or otherwise measuring the quantity of the second antibody bound to the sample. In certain embodiments, an increase in the amount of second antibody bound to the sample, as compared to a second sample lacking pFLT3, correlates with the presence of the FLT3-mediated hematological malignancy. In some embodiments, the method further comprises (f) comparing the amount of second antibody detected to a probability table and assigning a probability of the patient having a FLT3-mediated hematological malignancy to the amount of detected second antibody, whereby the patient is diagnosed with or not diagnosed with the hematological malignancy (see, e.g., Example 8). In certain embodiments, the diagnosis obtained from the various methods of detecting a FLT-mediated disease disclosed herein, may be used as part of a prognostic or diagnostic scoring system that takes into account one or more additional prognostic/diagnostic factors or measures selected from age, sex, blast count or percent blast count, hemoglobin or red blood cell count, lymphocyte or white blood cell count, platelet count, lactose dehydrogenase level, cytogenetics (e.g. chromosomal abnormalities), FLT3 genotype status including FLT3 ITD or FLT3 TKD mutation status, other AML associated prognostic mutation status such as MLL-PTD, CEBP1 or NPM1), other diagnostic results from biopsy, flow cytometry, immunohistochemistry such as ELISA and FISH, diagnosis of a leukemia or myelodysplastic disorder according to the French-American-British (FAB) classification and diagnosis of a leukemia or myelodysplastic disorder according to the World Health Organization (WHO) classification and other prognostic subclasses identified from gene expression profiling. For example, a recently developed system of grading severity of MDS, referred to as the International Prognostic Scoring System (IPSS), uses the sum of three factors: the percentage of blasts appearing in the bone marrow; the cytogenetic finding and the blood cell count; to score a patient's disease in terms of its risk, or in other words, used as a prognostic indicator. In one embodiment, the diagnosis of FLT3-mediated disease or a high blast count obtained from the methods disclosed herein, or alternatively, the measure of pFLT3 obtained from the methods disclosed herein may be used to as a fourth factor, to assigning a score that indicates the severity or prognosis of a particular MDS patient. In yet another embodiment, the method further comprising providing the prognostic or diagnostic score to a personnel, e.g., at a medical facility such as a doctor, nurse or other medical professional.

In a ninth aspect, provided herein are methods for diagnosing a patient having a high blast count, comprising detecting the amount of pFLT3 present in an in vivo sample of the patient (such as blood, blood lysate or bone marrow), and identifying the amount of pFLT3 present as corresponding to an amount of pFLT3 present in a sample having a high blast count. In another embodiment, provided herein is a method for diagnosing a patient having a high blast count comprising quantifying the amount of pFLT3 present in an in vivo sample of the patient (such as blood, blood lysate or bone marrow). In certain embodiments, the determination is performed using a sample directly from the patient, that is, without any sample processing beyond cell lysis prior to the method being performed. As another embodiment, provided herein are methods for diagnosing a patient with a high blast count, said method comprising: (a) contacting a sample (such as blood, blood lysate, plasma or bone marrow) from the patient with an immobilized first antibody that immunospecifically binds to human total FLT3; (b) removing unbound sample; (c) contacting the sample bound to the immobilized first antibody with a detectable second antibody, wherein the second antibody immunospecifically binds to human pFLT3, and wherein the second antibody immunospecifically binds to a different FLT3 epitope than the first antibody (such as an epitope comprising a phosphotyrosine); (d) removing unbound second antibody; and (e) detecting the presence of second antibody bound to the sample. In certain embodiments, the method further comprises (f) comparing the amount of second antibody detected to a correlation table assigning the amount of detected second antibody to a specific range of blast counts whereby the patient is diagnosed as having a blast count within the range. In one embodiment, the blast count is range is less then 1000 cells per microliter. In another embodiment, the blast count range is between 1000 and 10,000 cells per microliter. In yet another embodiment, the blast count range is greater than 10,000 cells per microliter. In one embodiment, the percent blast count is less than 5%. In another embodiment, the percent blast count range is between 5 and 10%. In one embodiment, the percent blast count range is less than 11%. In another embodiment, the percent blast count range is between 11 and 20%. In another embodiment, the percent blast count range is between 11 and 20%. In another embodiment, the blast count range is 20-29%. In another embodiment, the blast count range is 21-30%. In one embodiment, a percent blast count equal to or greater than 11% is considered a high blast count. In another embodiment, a percent blast count equal to or greater than 20% is considered a high blast count. In yet another embodiment, a percent blast count equal to or greater than 30% is considered a high blast count. The blast count or percent blast count may be determined by peripheral blood samples or bone marrow samples.

Sandwich-based immunoassay methods are well established in the art. See, e.g., U.S. Pat. Nos. 4,376,110 (David et al.); 4,016,043 (Schuurs et al.). Other related immunoassay formats and variations thereof which may be useful for carrying out the methods provided herein are well known in this field. See generally, E. Maggio (1980) Enzyme-Immunoassay (CRC Press, Inc., Boca Raton, Fla.); see also, e.g., U.S. Pat. Nos. 4,727,022 (Skold et al.); 4,659,678 (Forrest et al.); 4,376,110 (David et al.).

In certain embodiments of the various methods provided herein, the two or more of the steps are performed sequentially. In other embodiments of the methods provided herein, two or more of steps are performed in parallel (e.g., at the same time).

In some embodiments, a method is provided of classifying a patient for eligibility for FLT3-mediated disease therapy with, e.g., a FLT3 inhibitor comprising: (a) providing a tissue sample from a patient; (b) determining amount of pFLT3, e.g., using an ELISA method provided herein; and (c) classifying the patient as eligible to receive the therapy based on the amount of pFLT3 in the sample.

The sample subjected to the methods and kits provided herein can be any biological sample suspected of comprising pFLT3. In some embodiments, the sample is an in vivo sample, for example, biological fluid from a subject, e.g., a patient, such as a human patient. Non-limiting examples of such biological fluids include blood (e.g., human peripheral blood (HPB)), blood lysate, serum, blood plasma, fine needle aspirate, ductal lavage, spinal fluid, brain fluid, bone marrow, ascites fluid or any combination thereof. In other embodiments, the sample is taken from a biopsy tissue such as a tumor tissue from a subject or a thin layer cytological sample of other body tissue or organ. In certain embodiments, the sample comprises very low levels of FLT3 or pFLT3, such as levels that are otherwise undetectable by immunoprecipitation/immunoblotting (IP/Western) methods, for example, as those methods described in Smith et al.(2004) Blood 103:3669-3676, Brown et al. (2006) Leukemia 20:1368-1376, De Angelo et al. (2006) Blood 108:3674-3681, Stone et al. (2005) Blood 105:54-60, and/or Knapper et al. (2006) Blood 108(10): 3494-3503), and/or by immunoprecipitation/flow cytometry methods, such as those described in Ravandi et al. (2007) Leuk. Res. 31:791-797, Zheng et al.(2004) Blood 103: 267-274.

In certain embodiments, the in vivo sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

In certain embodiments, the methods presented herein are performed using samples directly from a subject or patient. That is, in such embodiments, beyond cell lysis, samples used in the methods are not processed, e.g., fractionated, purified, concentrated, or sorted according to cell type, prior to the methods being performed. In one embodiment, an unprocessed sample is a sample that is lysed but not fractionated, purified, concentrated or sorted according to cell type prior to the methods being performed. In another embodiment, an unprocessed sample is a sample that does not undergo any process for isolating blast cells.

In certain embodiments, the methods presented herein are performed using about 0.5 mg total sample protein, e.g., biological sample protein, such as in vivo sample protein. In other embodiments, the methods presented herein are performed using about 1.0 mg, 2.0 mg, 3.0 mg, 4.0 mg, 5.0 mg, 7.5 mg, 10.0 mg, 15.0 mg, or 20.0 mg total sample protein, e.g., biological sample protein, such as an in vivo sample protein. In other embodiments of the methods and kits provided herein, the sample is obtained from a patient suspected of having a FLT3-ITD mutation, a disease associated with constitutive FLT3 phosphorylation, or any other FLT3-mediated disease, such as AML, ALL and other types of leukemias and cancers. In yet another embodiment, the sample is prepared from a cell line expressing wild-type FLT3 (e.g., RS4:11) or FLT3 mutations such as FLT3-ITD (e.g., MV4:11). In certain embodiments, the sample is blood, bone marrow or other cell or tissue that has been subjected to lysis buffer, and the sample is in the form of blood lysate, or any other cell or tissue lysate. In certain embodiments, the sample is lysed with lysis buffer before being frozen at −70° C. In certain embodiments, the sample is treated with heparin and lysed before being frozen at −70° C. In certain embodiments, the sample is treated with heparin, lysed and tested fresh. In certain embodiments, the sample is lysed and tested fresh. In another embodiment, the sample is frozen at −70° C. and then lysed when thawed and then tested. In specific embodiments of the methods provided herein, the sample is human blood or lysate of human blood or bone marrow aspirate from a patient. In specific embodiments of the methods and kits provided herein, the sample is human blood, lysate of human blood, bone marrow aspirate or other biological fluid or tissue from a patent having a FLT3-mediated disease or disorder, or a symptom thereof. In some embodiments, the FLT3-mediated disease or disorder is a leukemia, such as AML or ACL. In other embodiments, the sample is human blood, lysate of human blood, or other biological fluid or tissue from a human patient having FLT3 mediated disease or disorder resulting from a FLT3-activating mutation. In certain embodiments, the FLT3-activating mutation is a FLT3-ITD mutation. In yet other embodiments, the sample is human blood, lysate of human blood, or other biological fluid or tissue from a patient that has been administered or will be administered human blood, lysate of human blood, or other biological fluid or tissue with a test compound, such as an antagonist of FLT3 phosphorylation (e.g., AC220). In certain embodiments, the lysis buffer is 1× cell lysis buffer which comprises 20 mM Tris, 137 nM NaCl, 10% glycerol, 1% NP-40, 0.1% SDS, 2 mM EDTA) supplemented with a protease/phosphatase inhibitor cocktail (PMSF used at 1x; Cat. No. P7626; and Sigma Cat. No. 11873580001, Roche, used at 1×). In another embodiment, the lysis buffer comprises 20 mM Tris, 137 nM NaCl, 10% glycerol, 1% NP-40, 0.1% SDS, 2 mM EDTA, 0.05 M NaF, supplemented with Complete Protease Inhibitor Cocktail Tablet (Cat. No. 11873580001, Roche, used at 1×) and Phosphatase Inhibitor Cocktail Set II (EMD_Calbiochem #624635 final used at 1×).

In certain embodiments of the methods and kits provided herein, the first antibody immunospecifically binds to the extracellular domain of human FLT3 (amino acids 27-543 of SEQ ID NO:1). In specific embodiments, the first antibody immunospecifically binds to the extracellular domain of the native form of human FLT3 (e.g., mouse anti-human total FLT3/Flk-2 capture monoclonal antibody, Cat. No. MAB8121; R&D Systems, Inc. (Minneapolis, Minn.)). In certain embodiments, the first antibody does not immunospecifically bind to one or more of the juxtamembrane domain (amino acids 572-603 SEQ ID NO:1), kinase insert region amino acids 711-780 of SEQ ID NO:1, intracellular domain amino acids 564-993 of SEQ ID NO:1 and C-terminus (amino acids 974-993 of SEQ ID NO:1). In specific embodiments, the first antibody is not a polyclonal antibody. In certain embodiments, the first antibody is a murine monoclonal antibody. In some embodiments of the methods provided herein, the method further comprises immobilizing the first antibody on a solid surface, such as a well of a multi-well plate by adding the first antibody to the solid surface, e.g., to each well of a multi-well plate, for example, at a concentration ranging from about 0.1 μg/mL to about 10 μg/mL. In one embodiment, about 0.25 ng to about 2.5 μg of antibody is added per well. In other embodiments, about 2.5 ng to about 250 ng of antibody is added per well. In certain embodiments of the methods and kits provided herein, about 15 ng to about 240 ng (e.g., about 15 ng, about 30 ng, about 60 ng, about 120 ng or about 240 ng), of the first antibody is immobilized on a well of a multi-well plate.

In some embodiments, the assay methods are carried out using solid phase assay formats. In certain embodiments, the assay methods provided herein are solid phase assays employing ECL detection. In specific embodiments of the methods and kits provided herein, the first antibody is immobilized on a solid surface. In certain embodiments, the first antibody is immobilized in a well of a plate with a plurality of wells, such as a multi-well plate or a multi-domain multi-well plate. The use of multi-well assay plates allows for the parallel processing and analysis of multiple samples distributed in multiple wells of a plate. Multi-well assay plates (also known as microplates or microtiter plates) can take a variety of forms, sizes and shapes (e.g., 96-, 384-, 1536-, or 9600-well plates; round- or flat-bottom multi-well plates). The methods provided herein, when carried out in standardized plate formats can take advantage of readily available equipment for storing and moving these plates as well as readily available equipment for rapidly dispensing liquids in and out of the plates (e.g., multi-well pipettes, plate washers and the like). Exemplary multi-well plate formats that can be used in the methods provided herein include those found on 96-well plates (12×8 array of wells), 384-well plates (24×16 array of wells) and 1536-well plate (48×32 array of well). Other formats that may be used in the methods provided herein include, but are not limited to, single or multi-well plates comprising a plurality of domains.

In certain embodiments of the methods and kits provided herein, the wells of the multi-well plate include integrated electrodes and can be used as solid support for immobilization of the first antibody, preferably configured so that the multi-well plate is able to support electrochemiluminescence (ECL). In certain embodiments, each well of the multi-well plate contains multiple zones or spots which allow multiple samples to be each individually contacted to one of the multiple zones or spots in a well of a multi-well plate. In some embodiments, the multi-well plate comprises carbon ink electrodes patterned on the bottom of the plate. In certain embodiments, the methods provided herein are carried out in parallel in multi-well plates.

The association or dissociation of labels from a surface (e.g., as occurs in a solid phase binding assay) can be measured in a washed or unwashed format. In an exemplary washed assay format, the surface of a well in a multi-well plate (e.g., the working electrode surface of a well in an ECL multi-well plate) is washed prior to contacting a solution (e.g., with a solution containing an ECL co-reactant, such as TPA that provides an appropriate environment for the induction of ECL from ECL labels) so as to remove unbound labeled reagents. Alternatively, in an exemplary unwashed assay format, the wash step may be omitted and, if appropriate, reagents such as ECL co-reactants are added without first removing unbound labeled reagents. The surface selectivity of ECL measurements, especially in measurements involving the use of electrodes as solid phase assay supports, allows ECL measurements to be carried out in washed or unwashed formats. In washed ECL assays, it is preferred that the ECL measurement be conducted within a short time period after the addition of the co-reactant solution to avoid loss of signal due to dissociation of the reagent in a binding interaction. In the unwashed assay, the timing is less important because free ligand remains in solution and the effect of the addition of the ECL co-reactant on the binding equilibrium is small. In this case, the ECL measurements can be conducted, for example, up to about one hour after addition of the ECL co-reactant solution with only a small change in signal.

The methods and kits provided herein utilize labeled secondary antibodies. Labels can be a substance used to directly or indirectly detect the antibody or other protein (e.g., streptavidin) that the label is attached to. In certain embodiments, the label is the antibody or other protein itself or, alternatively, the label may be covalently or non-covalently linked to the antibody or other protein. In certain embodiments of the invention, labels are used in order to follow or track the given antibody or other protein, for example, to determine its presence or amount. For example, immunoassays using labeled antibodies (e.g., antibodies labeled with ECL labels) can be used to detect and determine binding partners (analytes, such as FLT3 or pFLT3) bound by the antibody.

In some embodiments of the methods and kits provided herein, labels are used which may be detected directly, e.g., on the basis of a physical or chemical property of the label (e.g., optical absorbance, fluorescence, phosphorescence, chemiluminescence, electrochemiluminescence, refractive index, light scattering, radioactivity, magnetism, catalytic activity, or chemical reactivity). Examples of directly detectable labels include, radioactive labels, fluorescent labels, luminescent labels, enzyme labels, chemiluminescent labels, electrochemiluminescent labels, phosphorescent labels, light scattering or adsorbing particles (e.g., metal particles, gold colloids, silver colloids), magnetic labels and the like.

In other embodiments of the methods and kits provided herein, labels (e.g., biotin) are used which may be detected indirectly via interactions with species (e.g., streptavidin) comprising directly detectable labels (e.g., ruthenium (II) tri-bipyridine-(4-methylsulfonate) NHS ester). These indirectly detectable labels are, for example, binding species that readily allow the binding to a binding partner that is labeled with a directly detectable label. Examples of indirectly detectable labels include binding species, such as, antibodies, antigens, haptens, avidin, biotin, streptavidin, flourescein, nucleic acid sequences, nucleic acid analogue sequences, epitope tags (such as myc, FLAG, GST, MBP, V5), and digoxigenin. In specific embodiments, the measurement of the protein of interest in the sample (e.g., human pFLT3) is accomplished by immunospecific binding of an anti-pFLT3 antibody (e.g., an biotinylated anti-phosphotyrosine antibody) via a detectable label attached to the other member of the binding pair (e.g., a ruthenium (II) tri-bipyridine-(4-methylsulfonate) NHS ester-labeled streptavidin). In such assay methods, the number of binding interactions is correlated to the accumulation of labels on the solid phase; this accumulation of labels being measurable by a variety of techniques (e.g., fluorescence for fluorescence labels, enzyme activity for enzyme labels, or ECL for ECL labels.). In specific embodiments, the solid phase is an electrode material, the labels are ECL labels, and the accumulation of labels is measured via an ECL measurement.

In specific embodiments of the methods and kits provided herein, solid phase supports are used for purifying, immobilizing, or otherwise carrying out the solid phase assays. Examples of solid phases suitable for carrying out the methods of the invention include beads, particles, colloids, single surfaces, tubes, multiwell plates, microtitre plates, slides, membranes, gels and electrodes. When the solid phase is a particulate material (e.g., beads) it is, preferably, distributed in the wells of multi-well plates to allow for parallel processing of the solid phase supports. In specific embodiments of the methods and kits provided herein, the primary capture antibodies are immobilized on the solid phase supports, e.g., by non-specific adsorption, covalent attachment, or specific capture using an immobilized capture reagent that binds the primary antibody or other protein of interest. Immobilization may be accomplished by using proteins or assay reagents that are labeled with binding species that form binding pairs with immobilized capture reagents. Optionally, the antibody is immobilized on a solid phase, and contacted with a sample, such as a human blood sample or human blood lysate comprising, e.g., FLT3 or pFLT3, and the solid phase is washed. The wash step allows for the rapid purification of the FLT3 from other, potentially interfering, components of the sample. Optionally, the sample is treated, for example with a lysis buffer, prior to analysis.

In specific embodiments of the methods and kits provided herein, the solid phase supports are electrode surfaces integrated into the wells of multi-well plates. Such devices allow ECL measurements to be carried out in a high-throughput, highly parallel, fashion.

The multi-well assay plate can incorporate the electrode in one or more wells of the plate. The assay region (e.g., a given well of a multi-well plate) may also comprise additional electrodes. Preferably at least one electrode in an assay region (a well of a multi-well plate) is suitable for use as a working electrode in an electrode-induced luminescence (e.g., ECL) assay, at least one electrode is suitable for use as counter electrode in an electrode induced luminescence assay. Optionally, there is at least one electrode that is suitable for use as a reference electrode (e.g., in a three electrode electrochemical system). The surface of the working electrode in an electrode induced luminescence assay can be used for immobilization of one or more assay components, such as the primary anti-human FLT3 capture antibody. Electrodes used in the multi-well assay plates of the invention are typically non-porous, however, in some applications it is advantageous to use porous electrodes (e.g., mats of carbon fibers or fibrils, sintered metals, and metals films deposited on filtration membranes, papers or other porous substrates). These applications include those that employ filtration of solutions through the electrode so as to: increase mass transport to the electrode surface (e.g., to increase the kinetics of binding of molecules in solution to molecules on the electrode surface); capture particles on the electrode surface; and/or remove liquid from the well.

The multi-well assay plates that can be used in the methods and kits provided herein enable the performance of electrode induced luminescence-based assays inside one or more wells of a multi-well assay plate. Multi-well assay plates may include several elements including, for example, a plate top, a plate bottom, wells, working electrodes, counter electrodes, reference electrodes, dielectric materials, contact surfaces for electrical connections, conductive through-holes electrically connecting the electrodes and contact surfaces, adhesives, assay reagents, and identifying markings or labels. The wells of the plates may be defined by holes in the plate top; the inner walls of the holes in the plate top may define the walls of the well. The plate bottom can be affixed to the plate top (either directly or in combination with other components) and can serve as the bottom of the well. In some embodiments, the wells have at least one first electrode incorporated therein, and can also include at least one second electrode. In specific embodiments, the wells have at least one working electrode incorporated therein, and optionally also include at least one counter electrode. According to a certain embodiment, working, counter and, optionally, reference electrodes are incorporated into the wells and/or chambers. The assay plates are preferably flat, but may also be curved (not flat).

In specific embodiments, the multi-well assay plates greatly improve the speed and efficiency with which luminescence measurements are conducted. By incorporating the ability to induce electrode induced luminescence directly in a well of a multi-well assay plate, the invention overcomes an important limitation of the prior art, namely, the need to transfer the contents of a well in a standard multi-well plate (which lacks the features necessary for electrode induced luminescence tests) into a separate instrument that can conduct electrode induced luminescence-based measurements. In preferred examples of the present invention, multiple electrode induced luminescence (e.g., ECL) test measurements may be conducted in different wells of the same plate simultaneously. Such simultaneous operation dramatically increases the rate at which samples may be processed, eliminates cross-contamination of samples, significantly improves overall testing efficiency and enables the measurement of multiple analytes simultaneously. Because embodiments of the present invention incorporate electrodes into each well of the multi-well assay plates, it eliminates the need for a permanent, reusable measurement cell in the apparatus, which significantly reduces the cost and complexity of the apparatus. By measuring luminescence from sectors in a multi-well assay plate, the apparatus balances the desirable characteristics of rapid measurement times and high optical collection efficiencies.

Exemplary multi-well plates useful in the methods of the present invention are described in U.S. Pat. No. 6,977,722, which is incorporated herein by reference in its entirety. Multi-well plates useful in the methods provided herein are also commercially available from Meso Scale Discovery (Gaithersburg, Md.) (e.g., MULTI-ARRAY® or MULTI-SPOT® plates). In specific embodiments, the electrodes in such plates comprise a carbon composite electrode material such as carbon ink.

There are a number of commercially available instruments that use ECL for analytical measurements. Species that can be induced to emit ECL (ECL-active species) have been used as ECL labels. Examples of ECL labels include: (i.) organometallic compounds where the metal is from, for example, the noble metals of group VIII, including Ru-containing and Os-containing organometallic compounds such as the tris-bipyridyl-ruthenium (RuBpy) moiety and (ii.) luminol and related compounds. Species that participate with the ECL label in the ECL process are referred to as ECL co-reactants. Commonly used co-reactants include tertiary amines (e.g., see U.S. Pat. No. 5,846,485), oxalate, and persulfate for ECL from RuBpy and hydrogen peroxide for ECL from luminol (see, e.g., U.S. Pat. No. 5,240,863). The light generated by ECL labels can be used as a reporter signal in diagnostic procedures (see, e.g., Bard et al., U.S. Pat. No. 5,238,808). For instance, an ECL label can be covalently coupled to a binding agent such as an antibody (e.g., a secondary anti-pFLT3 antibody) or ligand; the participation of the binding reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL co-reactants). For more background on ECL, ECL labels, ECL assays and instrumentation for conducting ECL assays see U.S. Pat. Nos. 5,093,268; 5,147,806; 5,324,457; 5,591,581; 5,597,910; 5,641,623; 5,643,713; 5,679,519; 5,705,402; 5,846,485; 5,866,434; 5,786,141; 5,731,147; 6,066,448; 6,136,268; 5,776,672; 5,308,754; 5,240,863; 6,207,369 and 5,589,136 and Published PCT Nos. WO99/63347; WO00/03233; WO99/58962; WO99/32662; WO99/14599; WO98/12539; WO97/36931 and WO98/57154, which are herein incorporated by reference in their entirety.

Commercially available ECL instruments have demonstrated exceptional performance. They have become widely used for reasons including their excellent sensitivity, dynamic range, precision, and tolerance of complex sample matrices. The commercially available instrumentation uses flow cell-based designs with permanent reusable flow cells. Recently, ECL instrumentation has been disclosed that uses reagents immobilized on the electrode used to induce ECL (see, e.g., U.S. Pat. No. 6,207,369 and Published PCT Application No. WO98/12539). Multi-well plates having integrated electrodes suitable for such ECL measurements have also been recently disclosed (see, e.g., U.S. Pat. No. 6,977,722, which is incorporated herein by reference in its entirely). An exemplary commercially available ECL plate reader that can be used in the methods provided herein is the SECTOR® imager (Meso Scale Discovery, Gaithersburg, Md.), for example, in combination with MULTI-ARRAY® or MULTI-SPOT® multi-well plates (Meso Scale Discovery, Gaithersburg, Md.).

Thus, in certain embodiments, samples subjected to the methods and kits provided herein are detected and quantified using the MSD ECL detection system (Meso Scale Discovery; Gaithersburg, Md.). In specific embodiments, the MSD system employs a ruthenium (II) tri-bipyridine-(4-methylsulfonate) NHS ester (e.g., SULFO-TAG™, Meso Scale Discovery; Gaithersburg, Md.) label that emits light upon electrochemical activation. ECL measurements can be carried out using screen-printed carbon ink electrodes patterned on the bottom of specially designed 96-well multi-well or multi-domain multi-well plates, e.g., according to U.S. Pat. No 7,063,946 ((Kenten et al.) (e.g., MULTI-ARRAY and MULTI-SPOT® microplates, MSD). Each well of the plate has a patterned working electrode comprising 1, 4, 7, or 10 assay domains (approximately in the center of the well) that are exposed regions of electrode surface, which are defined by a patterned dielectric layer. The dielectric layer can be used to confine small volumes of liquid to specific assay domains. Each well also has two counter electrodes surfaces (e.g., approximately at two edges of the well). ECL from the labels on the surface of the carbon electrodes was induced and measured using an imaging plate reader (e.g., SECTOR® Imager 6000 and SECTOR® Imager 2400, Meso Scale Discovery, Gaithersburg, Md.).

As such, provided herein is a method for detecting the presence of human pFLT3 in a sample, e.g., an in vivo sample (e.g., human blood, human blood lysate or other biological fluid or tissue), said method comprising: (a) contacting the sample with an immobilized first antibody that immunospecifically binds to the extracellular domain of human FLT3 (amino acids 27-543 of SEQ ID NO:1), wherein the first antibody is immobilized on a multi-well plate or multi-domain multi-well plate comprising carbon ink electrodes on the bottom of the plate (e.g., MULTI-ARRAY® multi-well plate (Meso Scale Discovery, Gaithersburg, Md.); (b) removing unbound sample; (c) contacting the sample bound to the immobilized first antibody with a mixture of biotinylated second antibody and labeled streptavidin, wherein the second antibody immunospecifically binds to human pFLT3, and wherein the second antibody immunospecifically binds to a different FLT3 epitope than the first antibody (such as an epitope comprising a phosphotyrosine) and wherein the label comprises ruthenium (II) tri-bipyridine-(4-methylsulfonate) NHS ester (e.g., SULFO-TAG™, Meso Scale Discovery (Gaithersburg, Md.); (d) removing unbound second antibody and unbound labeled streptavidin; and (e) detecting the presence of second antibody bound to the sample by ECL of the labeled-streptavidin on the surface of the carbon electrodes (e.g., using e.g., SECTOR® Imager 6000 and SECTOR® Imager 2400, Meso Scale Discovery, Gaithersburg, Md.); wherein a greater amount of second antibody bound to the sample, as compared to a control sample having no pFLT3, indicates the presence of human pFLT3 in the sample. In certain embodiments, the first antibody is a mouse anti-human total FLT3/Flk-2 capture monoclonal antibody (Cat. No. MAB8121, R&D Systems, Inc. (Minneapolis, Minn.)) and the second antibody is in a mixture of 2 μg/ml biotinylated mouse anti-phospho-tyrosine (4G10®) mAb (Cat No. 16-103; Millipore Corp.; Billerica, Mass.) and 2 μg/ml SULFO-TAG™-labeled streptavidin (Cat No. R32AD; Meso Scale Discovery; Gaithersburg, Md.).

Antibodies for use in the methods and kits provided herein include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′)₂ fragments, disulfide-linked Fvs (sdFv), anti-idiotypic (anti-Id) antibodies, and epitope-binding fragments of any of the above. In specific embodiments, the first and/or second antibody of the methods provided herein is a monoclonal antibody.

In particular, antibodies for use in the methods and kits provided herein include immunoglobulin molecules and immunologically active portions of immunoglobulin molecules, i.e., molecules that contain an antigen binding site that immunospecifically binds to a human FLT3, pFLT3, and/or a phosphotyrosine. The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule. In specific embodiments, the first and/or second antibody of the methods provided herein is an IgG antibody.

Variants and derivatives of antibodies include antibody fragments that retain the ability to specifically bind to an epitope. Preferred fragments include Fab fragments (an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)₂ (two Fab′ molecules joined by interchain disulfide bonds in the hinge regions of the heavy chains; the Fab′ molecules may be directed toward the same or different epitopes); a bispecific Fab (a Fab molecule having two antigen binding domains, each of which may be directed to a different epitope); a single chain Fab chain comprising a variable region, also known as, a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present. In certain embodiments, the antibody to be used with the invention comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs see Pluckthun in The Pharmacology of Monoclonal Antibodies, vol. 113, Rosenburg and Moore eds. Springer-Verlag, New York, pp. 269-315 (1994).

The antibodies of the invention may be from any animal origin including birds and mammals (e.g., human, murine, donkey, sheep, rabbit, goat, guinea pig, camel, horse, or chicken). In certain embodiments, the antibodies of the invention are human or humanized monoclonal antibodies. As used herein, “human” antibodies include antibodies having the amino acid sequence of a human immunoglobulin and include antibodies isolated from human immunoglobulin libraries or from mice that express antibodies from human genes.

In certain embodiments of the methods provided herein, the first antibody, or antigen binding fragment thereof, immunospecifically binds to the extracellular domain (amino acids 27-543 of SEQ ID NO:1) of human FLT3.

In some embodiments, the first antibody, or antigen binding fragment thereof, immunospecifically binds to the native form of human FLT3, such as the extracellular domain of native human FLT3. In some embodiments, the antibodies provided herein bind to a human FLT3 epitope that is a three-dimensional surface feature of a human FLT3 polypeptide. A region of a human FLT3 polypeptide contributing to an epitope may be contiguous amino acids of the polypeptide or the epitope may come together from two or more non-contiguous regions of the polypeptide. A FLT3 epitope may be present in (a) the dimeric form (“a dimeric FLT3 epitope”) of human FLT3, (b) the monomeric form (“a monomeric FLT3 epitope”) of human FLT3, (c) both the dimeric and monomeric form of human FLT3, (d) the dimeric form, but not the monomeric form of human FLT3, or (e) the monomeric form, but not the dimeric form of human FLT3. In specific embodiments, the first antibody immunospecifically binds to an epitope in the extracellular domain of the native form of human FLT3 (amino acids 27-543 of SEQ ID NO:1).

For example, in some embodiments, the epitope is only present or available for binding in the dimeric (native) form, but is not present or available for binding in the monomeric (denatured) form by an anti-FLT3 antibody. In other embodiments, the FLT3 epitope is a linear feature of the FLT3 polypeptide (e.g., in a dimeric form or monomeric form of the FLT3 polypeptide). Antibodies provided herein may immunospecifically bind to (a) an epitope of the monomeric form of human FLT3, (b) an epitope of the dimeric form of human FLT3, (c) an epitope of the monomeric but not the dimeric form of human FLT3; (d) an epitope of the dimeric but not the monomeric form of human FLT3; or (e) both the monomeric form and the dimeric form of human FLT3.

In some embodiments, the first antibody, or antigen binding fragment thereof, immunospecifically binds to one or more of the juxtamembrane domain of human FLT3 (amino acids 572-603 of SEQ ID NO:1), the kinase insert domain of human FLT3 (amino acid residues 740-757 of SEQ ID NO:1), the intracellular domain of human FLT3 (amino acids 564-993 of SEQ ID NO:1), the C-terminus of human FLT3 (amino acids 974-993 of SEQ ID NO:1), or any combination thereof. In other embodiments, the first antibody, or antigen binding fragment thereof, does not immunospecifically bind to one or more of the juxtamembrane domain of human FLT3 (amino acids 572-603 of SEQ ID NO:1), the kinase insert domain of human FLT3 (amino acid residues 740-757 of SEQ ID NO:1), the intracellular domain of human FLT3 (amino acids 564-993 of SEQ ID NO:1), the C-terminus of human FLT3 (amino acids 974-993 of SEQ ID NO:1), or any combination thereof.

In some embodiments of the methods provided herein, the first antibody and/or the second antibody is a monoclonal antibody. In other embodiments, the first antibody and/or the second antibody is not a polyclonal antibody.

The methods provided herein generally involve the detection of secondary antibody, which may or may not require labels (e.g., refractive index based techniques such as surface plasmon resonance). In certain embodiments of the methods and kits provided herein, the second antibody immunospecifically binds to a phosphorylated tyrosine (phosphotyrosine) of human pFLT3. In certain embodiments, the second antibody immunospecifically binds to one or more of phosphorylated tyrosine residues at positions 589, 591, 597, 599, 726, 842, and 955 of human pFLT3 (SEQ ID NO: 1) (e.g., anti-phosphotyrosine, clone 4G10®, biotin conjugate; anti-phosphotyrosine, PY-20 SULFO-TAG™, Meso Scale Discovery (Gaithersburg, Md.)). In other embodiments, the second antibody does not immunospecifically bind to one or more of phosphorylated tyrosine residues at positions 589, 591, 597, 599, 726, 842, and 955 of human pFLT3 (SEQ ID NO: 1). In certain embodiments, the second antibody does not immunospecifically bind solely to phosphorylated tyrosine residues at positions 589 and/or 591 of human pFLT3 (SEQ ID NO:1) (see, e.g., anti-Tyr591 (Cell Signaling Technologies); anti-Tyr589 as described in U.S. Pat. No. 7,183,385).

In certain embodiments, the second antibody comprises a detectable label. In some embodiments, the labeled second antibody comprises a biotin, radionuclide, enzyme, substrate, fluorescent marker, chemiluminescent marker, or ruthenium (II) tri-bipyridine-(4-methylsulfonate) NHS ester label. In specific embodiments, the second antibody is biotinylated. In certain embodiments, the second antibody is a murine monoclonal antibody. In certain embodiments, the second antibody is contacted with the sample at a concentration ranging from about 0.1 μg/mL to about 10 μg/mL. In one embodiment, about 0.25 ng to about 2.5 μg of antibody is added per well. In other embodiments, about 2.5 ng to about 250 ng of antibody is added per well.

In some embodiments of the methods and kits provided herein, the first antibody is a mouse anti-human FLT3 IgG mAb (Cat. No. MAB8121; R&D Systems, Inc.; Minneapolis, Minn.). In some embodiments, the first antibody is at 120 ng per well. In other embodiments, the second antibody is biotinylated goat anti-human FLT3 IgG Ab (Cat No. BAF812; R&D Systems, Inc.; Minneapolis, Minn.). In some embodiments the second antibody is at 5 ng per well. In certain embodiments, the second antibody is a mixture of biotinylated goat anti-human FLT3 IgG Ab (Cat No. BAF812; R&D Systems, Inc.; Minneapolis, Minn.) at 5 ng per well (0.2 μg/ml in 25 μl volume) plus SULFO-TAG™-labeled streptavidin (Cat No. R32AD; Meso Scale Discovery; Gaithersburg, Md.) at 25 ng per well (1 μg/ml in 25 μl volume).

In some embodiments of the methods provided herein, the second antibody is biotinylated, and the method further comprises contacting the biotinylated second antibody with a labeled streptavidin. Certain embodiments of the methods provided herein comprise contacting a mixture comprising the second biotinylated antibody and the labeled streptavidin to the sample bound to the first antibody. In certain embodiments, the streptavidin is labeled with ruthenium (II) tri-bipyridine-(4-methylsulfonate) NHS ester. In one embodiment, the labeled streptavidin is detected by ECL of the ruthenium (II) tri-bipyridine-(4-methylsulfonate) NHS ester-labeled streptavidin on the surface of carbon ink electrodes patterned on the bottom of a multi-well plate, such as those described elsewhere herein (e.g., MULTI-ARRAY® or MULTI-SPOT® plates commercially available from Meso Scale Discovery (Gaithersburg, Md.)).

In certain embodiments, the dynamic range of the assay methods provided herein (e.g., wherein the sample is blood, blood lysate or bone marrow aspirate) corresponds to a signal-to-noise ratio of between about 2-100 (e.g., a signal that is between about 2-fold and 100-fold above background), such as between about 5-100, between about 10-100, between about 20-100, between about 30-100, between about 40-100, between about 50-100, between about 60-100, between about 70-100, or between 80-100. In certain embodiments, the dynamic range of the assay methods provided herein (e.g., wherein the sample is blood, blood lysate or bone marrow aspirate) corresponds to a signal-to-noise ratio of between about 1-70, such as between about 5-70, between about 10-70, between about 20-70, between about 30-70, between about 40-70, between about 50-70, or between about 60-70. In one embodiment, the dynamic range of the assay methods provided herein where the sample is blood or blood lysate corresponds to a signal-to-noise ratio of between about 10-30. In another embodiment, the dynamic range of the assay methods provided herein, where the sample is blood or blood lysate, corresponds to a signal-to-noise ratio of between about 10-20.

In certain embodiments, the dynamic range of the assay methods provided herein, where the sample is a cell lysate sample, corresponds to a signal-to-noise ratio of between about 10 and 1×10⁶, for example, between about 10, 100, 1000, or 1×10⁵ and about 100, 1000, 1×10⁵ or 1×10⁶. In one embodiment, the dynamic range of the assay methods provided herein, where the sample is a cell lysate, corresponds to a signal-to-noise ratio of between about 50-100, between about 60-100 or between about 70-100.

It is noted that any combination of the above-listed embodiments, for example, with respect to sample, patient populations, solid phase immobilization, first antibody, second antibody, labels and the like, are also contemplated in relation to any of the various methods and/or kits provided herein.

In addition, the methods and kits provided herein may be used to detect or otherwise monitor pFLT3 levels in species other than human, as the high degree of homology between human FLT3 and other species (e.g., mouse, dog, cat) is well known in the art.

In certain embodiments, the methods for detecting human pFLT3 provided herein are used to determine the relative amounts (e.g., high, medium or low) of pFLT3 in a sample from a patient, wherein the amount of pFLT3 in the sample correlates with the presence of a FLT3-activating mutation, such as FLT3-ITD, in the patient. Such methods can be used as a substitute for genotyping the patient for a FLT3-activating mutation.

In one embodiment, the determination of high, medium or low levels of pFLT3 is based in part on the type of tissue sample tested as some tissues may be more enriched with FLT3 than others, on the type of signal strength obtained by the assay detection method, and/or based in part on the signal distribution observed among the sample of patients. In one specific embodiment, a low level of pFLT3 in a sample, e.g., an in vivo sample, such as a blood, blood lysate or other bodily fluid or tissue, corresponds to a signal less than 500 obtained from the MSD detection method provided herein (see, e.g., Example 8), and a medium to high level of pFLT3 in the sample, corresponds to a signal at or above 500. In certain embodiments, a signal equal to or greater than 500 and equal to or less than 1000 obtained from the MSD detection method is considered to be a medium level of pFLT3. In specific embodiments, any signal greater than 1000 obtained from the MSD detection method is considered to be a high level of pFLT3 in the sample.

In another embodiment, the determination of high, medium and low levels of pFLT3 is based in part on the type of tissue tested, the signal strength obtained by the assay detection method, and/or on the signals observed among a sampling of patients, and is guided, in part, by signals observed in those patients that have been definitively genotyped as having a FLT3-activating mutation, such as FLT3-ITD.

For example if information on percent FLT3-ITD is available, the signal associated with a certain percentage of FLT3-ITD, for example 50% FLT3-ITD, may be used as a dividing line between medium and high levels of pFLT3.

Alternatively, statistical analysis may be used to measure the significance of the correlation and to determine which range of assay signal strength should be designated low, medium or high level of pFLT3. For example, a statistical software R may be used, optionally in conjunction with a data analysis tool such as Spotfire (TIBCO Software Inc.) to measure the correlation and to make statistically significant groupings of low, medium and high levels of FLT3. In one embodiment, experiments are conducted with a larger population sample comprising leukemic patients and healthy patients so that a baseline measure of total FLT3 and pFLT3 may be established. Other experiments would be conducted to determine what factors contribute to variability of results in order to be able to recognize and minimize variability in the sample set. For example, tissue type may impact the range of signal obtained from those samples because some tissues may be more enriched with FLT3 and pFLT3 than others. A correlation analysis might be done for each tissue type, or, if there is a mathematical relationship between the signal obtained for two different types of tissue, mathematical adjustments may be made to handle different tissue types in a single correlation analysis. Other experiments could be conducted to determine whether other factors such as sample handling, for example, could contribute to variability in the FLT3 and pFLT3 measurements. For example, if the type of lysis buffer used, or blood lysis method or blood storage conditions are found to impact FLT3 and pFLT3 measurements, a protocol may be devised to minimize variability.

Another embodiment provided herein is a method of monitoring or otherwise tracking the evolution of a patient's FLT3-mediated disease or disorder, such as leukemia. Leukemia is a continually evolving disease in which mutations are often acquired and less commonly lost. In certain cases, leukemic cells express both mutant and wild type FLT3, and the ratio of mutant to wild type FLT3 may change over the course of the leukemia. FLT3-activating mutations, such as FLT3-ITD, are often acquired during AML evolution or during the evolution of MDS to AML. The ratio of mutant FLT3 to wild type may also evolve, with a high ratio of mutant to wild type, which is associated with poor prognosis (Whitman et al. Canc Res 2001 61, 7233-7239). Thus, the methods to measure the pFLT3 levels provided herein may be used as a as a surrogate for genotyping to track the appearance of FLT3-activating mutations, such as FLT3-ITD, and/or to track changes in the mutant to wild type FLT3 ratios by monitoring or otherwise tracking pFLT3 levels in the patient. In some embodiments, a patient whose sample (e.g., an in vivo sample, such as a blood, blood lysate or other bodily fluid or tissue) generates an initial pFLT3 signal less than 500 obtained from the MSD detection method provided herein is monitored. In some embodiments, a patient whose sample generates an initial pFLT3 signal at or above 500 obtained from the MSD detection method provided herein is monitored. In some embodiments, a patient whose sample generates an initial pFLT3 signal of between 500 and 1000 obtained from the MSD detection method provided herein is monitored.

In one embodiment, the methods of determining pFLT3 levels provided herein are used for molecular classification, either alone or in conjunction with other methods of classification based, for example, on blast count, blast percentage, blast morphology, karyotyping (including determining chromosomal abnormalities such as t(8;21) or t(8;12)(q22;q22), inv(16) or inv(16)(p13q22), t(16;16) or t(16;16)(p13;q22) and t(15;17) or t(15;17)(q22;q12)), genotyping (including detection of genetic abnormalities such as the FLT3-ITD mutation or the D835 point mutation), gene expression profiling, or any combination thereof. In one embodiment, a patient suspected of having a hematological malignancy is diagnosed using genotyping or cytogenetics (i.e. by determining chromosomal abnormalities) and pFLT3 measurement.

In certain embodiments, the methods provided herein further comprises providing results from the assay to personnel, e.g., at a medical facility, such as a doctor, nurse or other medical professional. In other embodiments, the method provided herein further comprises providing therapeutic options to personnel, e.g., at a medical facility, such as a doctor, nurse or other medical professional.

Kits for carrying out the methods provided herein are also contemplated. The kits provide useful tools to help diagnose a FLT3-activating mutation and/or a FLT3-mediated disease or disorder, such as a leukemia (e.g., AML or ALL), monitor the possible progression of the FLT3-mediated disease or disorder, and/or assess the risk of the subject developing a FLT3-mediated disease or disorder, such as a FLT3-mediated disease or disorder resulting from a FLT3 mutations.

Thus, in a tenth aspect, provided herein are kits for detecting human pFLT3 in a sample (e.g., a bodily fluid, such as blood, or other tissue sample) of a patient, comprising: (a) a first antibody that immunospecifically binds to human total FLT3, optionally immobilized on a solid surface; (b) a detectable second antibody, wherein the second antibody immunospecifically binds to human pFLT3, and wherein the second antibody immunospecifically binds to a different FLT3 epitope than the first antibody (such as an epitope comprising a phosphotyrosine). Any combination of the above-listed embodiments, for example, with respect to sample, patient populations, solid phase immobilization, first antibody, second antibody, labels and the like, are also contemplated embodiments in connection with the kits provided herein. In specific embodiments, the first antibody is immobilized on a multi-well plate or multi-domain multi-well plate comprising carbon ink electrodes on the bottom of the plate (e.g., MULTI-ARRAY®, Meso Scale Discovery, Gaithersburg, Md.). In one embodiment, the kit for detecting human pFLT3 in an in vivo sample comprises (a) a first antibody that immunospecifically binds to the extracellular domain of human FLT3, optionally immobilized on a solid surface and (b) a detectable second antibody, wherein the second antibody immuospecifically binds to a phosphorylated tyrosine residues of human FLT3 wherein the second antibody immunospecifically binds to a different FLT3 epitope than the first antibody. In certain embodiments, the second antibody is a biotinylated anti-phospho-tyrosine antibody. In a specific embodiment, the kit for detecting human pFLT3 in an in vivo sample comprises (a) a mouse anti-human FLT3 IgG mAb (Cat No. MAB8121; R&D Systems, Inc.; Minneapolis, Minn.) optionally immobilized on a MSD plate (Meso Scale Discovery, Gaithersburg, Md.) and (b) a biotinylated mouse anti-phospho-tyrosine mAb 4G10® (Cat No. 16-103; Millipore Corp.; Billerca, Mass.). In another embodiment, the kit further comprises SULFO-TAG™-labeled streptavidin (Cat No. 832AD; Meso Scale Discovery; Gaithersburg, Md.).

In a specific embodiment, provided herein is a for detecting the presence of human pFLT3 in a sample, e.g., an in vivo sample (e.g., human blood, human blood lysate or other biological fluid or tissue), said kit comprising: (a) a first antibody that immunospecifically binds to the extracellular domain of human FLT3 (amino acids 27-543 of SEQ ID NO:1), wherein the first antibody is either already immobilized on a multi-well plate or multi-domain multi-well plate comprising carbon ink electrodes on the bottom of the plate (e.g., MULTI-ARRAY® multi-well plate (Meso Scale Discovery, Gaithersburg, Md.) or is provided separately with the multi-well plate; (b) a biotinylated second antibody, wherein the second antibody immunospecifically binds to human pFLT3, and wherein the second antibody immunospecifically binds to a different FLT3 epitope than the first antibody (such as an epitope comprising a phosphotyrosine); and (c) a labeled-streptavidin, wherein the label comprises ruthenium (II) tri-bipyridine-(4-methylsulfonate) NHS ester (e.g., SULFO-TAG™, Meso Scale Discovery (Gaithersburg, Md.). In some embodiments of the methods and kits provided herein, the first antibody is a mouse anti-human FLT3 IgG mAb (Cat. No. MAB8121; R&D Systems, Inc.; Minneapolis, Minn.). In some embodiments, the first antibody is at 120 ng per well. In other embodiments, the second antibody is biotinylated goat anti-human FLT3 IgG Ab (Cat No. BAF812; R&D Systems, Inc.; Minneapolis, Minn.). In some embodiments the second antibody is at 5 ng per well. In certain embodiments, the second antibody is a mixture of biotinylated goat anti-human FLT3 IgG Ab (Cat No. BAF812; R&D Systems, Inc.; Minneapolis, Minn.) at 5 ng per well (0.2 μg/ml in 25 μl volume) plus SULFO-TAG™-labeled streptavidin (Cat No. R32AD; Meso Scale Discovery; Gaithersburg, Md.) at 25 ng per well (1 μg/ml in 25 μl volume).

The kits provided herein can be used in identifying a test compound that is an agonist or antagonist of FLT3 phosphorylation. These kits can also be used to perform methods provided herein prior to preliminary blood evaluation or surgical surveillance procedures, and may be used as a surrogate assay for FLT3 genotyping, e.g., for a FLT3-activating mutation, such as a FLT3-ITD mutation. The kits provided herein can also be used to perform the methods provided herein for determining if a patient has a FLT3-activating disease, and/or to monitor or otherwise track a FLT3-mediated disease in patient. The kits can be packaged in any suitable manner, typically with the various parts, in a suitable container along with instructions for use.

In certain embodiments, the kits may further comprise, where necessary, other members of the signal-producing system of which the detectable group is a member (e.g., enzyme substrates), agents for reducing the background interference in a test, control reagents, apparatus for conducting a test, and the like.

The following examples are offered by way of illustration, and not by way of limitation.

### EXAMPLES

### Example 1

### Development of Total and pFLT3 ELISA Assays

A large number of potential total FLT3 and pFLT3 capture and detection antibodies are available, as are a large number of detection techniques. In view of the different elements that could be chosen and varied, this could give rise to a very large number of assay combinations. Given how extremely low the level of pFLT3 appears to be in in vivo samples such as blood, prior to the work described herein, it was unpredictable, which, if any, of the theoretical assay combinations could successfully detect pFLT3 in an in vivo sample, particularly assays performed using only a minimal amount of sample protein.

Exemplary total and pFLT3 ELISA assays were developed and optimized using different combinations of first (capture) and second (detection) antibodies on MSD 96-well plates ((Meso Scale Discovery; Gaithersburg, Md.). The initial assays were developed and optimized using MV4:11 cell lysates. The following thirteen antibody combinations were tested. Capture and detection antibodies tested in various combinations are further summarized below in Tables 1 and 2, respectively:

Group 1: Capture: Mouse anti-human FLT3 IgG mAb (Cat. No. MAB8121; R&D Systems, Inc.; Minneapolis, Minn.) at 60 ng, 120 ng or 240 ng per well. Detection: Rabbit anti-human FLT3 IgG pAb (Cat No. SC-479; Santa Cruz Biotechnology, Inc.; Santa Cruz, Calif.) at 50 ng per well plus SULFO-TAG™-labeled goat anti-rabbit Ab (Cat No. R32AB; Meso Scale Discovery; Gaithersburg, Md.) at 25 ng per well.

Group 2: Capture: Mouse anti-human FLT3 IgG mAb (Cat. No. MAB8121; R&D Systems, Inc.; Minneapolis, Minn.) at 60 ng, 120 ng or 240 ng per well. Detection: Biotinylated goat anti-human FLT3 IgG Ab (Cat No. BAF812; R&D Systems, Inc.; Minneapolis, Minn.) at 25 ng per well plus SULFO-TAG™-labeled streptavidin (Cat No. R32AD; Meso Scale Discovery; Gaithersburg, Md.) at 25 ng per well.

Group 3: Capture: Mouse anti-human FLT3 IgG mAb (Cat. No. MAB8121; R&D Systems, Inc.; Minneapolis, Minn.) at 60 ng, 120 ng or 240 ng per well. Detection: SULFO-TAG™-labeled anti-phospho-tyrosine (PY-20) Ab (Cat No. R32AD; Meso Scale Discovery; Gaithersburg, Md.) at 75 ng per well.

Group 4: Capture: Mouse anti-human FLT3 IgG mAb (Cat. No. MAB8121; R&D Systems, Inc.; Minneapolis, Minn.) at 60 ng, 120 ng or 240 ng per well. Detection: Biotinylated mouse anti-phospho-tyrosine (4G10R) mAb (Cat No. 16-103; Millipore Corp.; Billerica, Mass.) at 50 ng per well plus SULFO-TAG™-labeled streptavidin (Cat No. R32AD; Meso Scale Discovery; Gaithersburg, Md.) at 50 ng per well.

Group 5: Capture: Rabbit anti-human FLT3 IgG pAb (Cat No. SC-479; Santa Cruz Biotechnology, Inc.; Santa Cruz, Calif.) at 60 ng, 120 ng or 240 ng per well. Detection:

Biotinylated goat anti-human FLT3 IgG Ab (Cat No. BAF812; R&D Systems, Inc.; Minneapolis, Minn.) at 50 ng per well plus SULFO-TAG™-labeled streptavidin (Cat No. R32AD; Meso Scale Discovery; Gaithersburg, Md.) at 50 ng per well.

Group 6: Capture: Rabbit anti-human FLT3 IgG pAb (Cat No. SC-479; Santa Cruz Biotechnology, Inc.; Santa Cruz, Calif.) at 15 ng, 30 ng, 60 ng, 120 ng or 240 ng per well.

Detection: SULFO-TAG™-labeled anti-phospho-tyrosine (PY-20) Ab (Cat No. R32AP; Meso Scale Discovery; Gaithersburg, Md.) at 75 ng per well.

Group 7: Capture: Rabbit anti-human FLT3 IgG pAb (Cat No. SC-479; Santa Cruz Biotechnology, Inc.; Santa Cruz, Calif.) at 15 ng, 30 ng, 60 ng, 120 ng or 240 ng per well. Detection: Biotinylated mouse anti-phospho-tyrosine (4G10®) mAb (Cat No. 16-103; Millipore Corp.; Billerica, Mass.) at 50 ng per well plus SULFO-TAG™-labeled streptavidin (Cat No. R32AD; Meso Scale Discovery; Gaithersburg, Md.) at 50 ng per well.

Group 8: Capture: Rabbit anti-human FLT3 IgG pAb (Cat No. SC-479; Santa Cruz Biotechnology, Inc.; Santa Cruz, Calif.) at 60 ng per well. Detection: SULFO-TAG™-labeled anti-phospho-tyrosine (PY-20) Ab (Cat No. R32AP; Meso Scale Discovery; Gaithersburg, Md.) at 37.5 ng per well.

Group 9: Capture: Rabbit anti-human FLT3 IgG pAb (Cat No. SC-340; Santa Cruz Biotechnology, Inc.; Santa Cruz, Calif.) at 60 ng per well. Detection: SULFO-TAG™-labeled anti-phospho-tyrosine (PY-20) Ab (Cat No. R32AP; Meso Scale Discovery; Gaithersburg, Md.) at 37.5 ng per well.

Group 10: Capture: Rabbit anti-human FLT3 IgG pAb (Cat No. SC-20733; Santa Cruz Biotechnology, Inc.; Santa Cruz, Calif.) at 60 ng per well. Detection: SULFO-TAG™-labeled anti-phospho-tyrosine (PY-20) Ab (Cat No. R32AP; Meso Scale Discovery; Gaithersburg, Md.) at 37.5 ng per well.

Group 11: Mouse anti-pFLT3 (Tyr591) mAb (Cat No. 3466, Cell Signaling Technology, Inc.; Danvers, Mass.) at 60 ng per well. Detection: Biotinylated goat anti-human FLT3 IgG Ab (Cat No. BAF812; R&D Systems, Inc.; Minneapolis, Minn.) at 25 ng per well plus SULFO-TAG™-labeled streptavidin (Cat No. R32AD; Meso Scale Discovery; Gaithersburg, Md.) at 25 ng per well.

Group 12: Capture: Mouse anti-human FLT3 IgG mAb (Cat. No. MAB8121; R&D Systems, Inc.; Minneapolis, Minn.) at 60 ng per well. Detection: SULFO-TAG™-labeled anti-phospho-tyrosine (PY-20) Ab (Cat No. R32AP; Meso Scale Discovery; Gaithersburg, Md.) at 37.5 ng per well.

Group 13: Capture: Mouse anti-human FLT3 IgG mAb (Cat. No. MAB8121; R&D Systems, Inc.; Minneapolis, Minn.) at 60 ng per well. Detection: Biotinylated goat anti-human FLT3 IgG Ab (Cat No. BAF812; R&D Systems, Inc.; Minneapolis, Minn.) at 25 ng per well plus SULFO-TAG™-labeled streptavidin (Cat No. R32AD; Meso Scale Discovery; Gaithersburg, Md.) at 25 ng per well.

TABLE 1
Capture Antibodies.

| | Capture Ab | Ab conc. used | Amount of Ab |
|---|---|---|---|
| Vendor: Cat, No. | stock conc. | (stock in TBS) | in 30 μl |
| R&D: MAB8121 | 720 μg/ml | 2 μg/ml | 60 ng |
| R&D: MAB8121 | 720 μg/ml | 4 μg/ml | 120 ng |
| R&D: MAB8121 | 720 μg/ml | 8 μg/ml | 240 ng |
| R&D: MAB8121 | 720 μg/ml | 2 μg/ml | 60 ng |
| R&D: MAB8121 | 720 μg/ml | 4 μg/ml | 120 ng |
| R&D: MAB8121 | 720 μg/ml | 8 μg/ml | 240 ng |
| R&D: MAB8121 | 720 μg/ml | 2 μg/ml | 60 ng |
| R&D: MAB8121 | 720 μg/ml | 4 μg/ml | 120 ng |
| R&D: MAB8121 | 720 μg/ml | 8 μg/ml | 240 ng |
| R&D: MAB8121 | 720 μg/ml | 2 μg/ml | 60 ng |
| R&D: MAB8121 | 720 μg/ml | 4 μg/ml | 120 ng |
| R&D: MAB8121 | 720 μg/ml | 8 μg/ml | 240 ng |
| Santa Cruz: SC-479 | 200 μg/ml | 2 μg/ml | 60 ng |
| Santa Cruz: SC-479 | 200 μg/ml | 4 μg/ml | 120 ng |
| Santa Cruz: SC-479 | 200 μg/ml | 8 μg/ml | 240 ng |
| Santa Cruz: SC-479 | 200 μg/ml | 0.5 μg/ml | 15 ng |
| Santa Cruz: SC-479 | 200 μg/ml | 1 μg/ml | 30 ng |
| Santa Cruz: SC-479 | 200 μg/ml | 2 μg/ml | 60 ng |
| Santa Cruz: SC-479 | 200 μg/ml | 4 μg/ml | 120 ng |
| Santa Cruz: SC-479 | 200 μg/ml | 8 μg/ml | 240 ng |
| Santa Cruz: SC-479 | 200 μg/ml | 0.5 μg/ml | 15 ng |
| Santa Cruz: SC-479 | 200 μg/ml | 1 μg/ml | 30 ng |
| Santa Cruz: SC-479 | 200 μg/ml | 2 μg/ml | 60 ng |
| Santa Cruz: SC-479 | 200 μg/ml | 4 μg/ml | 120 ng |
| Santa Cruz: SC-479 | 200 μg/ml | 8 μg/ml | 240 ng |
| Santa Cruz: SC-20733 | 200 μg/ml | 2 μg/ml | 60 ng |
| Cell Signaling: 3466 | 450 μg/ml | 2 μg/ml | 60 ng |

TABLE 2
Detection Antibodies.

| Vendor: | Detection Ab stock | Final Ab conc. used (in Ab | |
|---|---|---|---|
| Cat. No. | concentration | dilution buffer A) | Volume per well in 25 μl |
| Santa Cruz: | 200 μg/ml SC479 + 500 μg/ml | 2 μg/ml SC479 + 1 μg/ml anti | 50 ng SC479 + |
| SC-479 | SULFO-TAG ®-anti rabb | species | 25 ng anti-species |
| R&D: | 5.4 μg/ml BAF812 + 500 μg/ml | 1 μg/ml (or 0.2 μg/ml) detec. | 25 ng (or 5 ng) detec Ab + |
| BAF812 | SULFO-TAG ® streptavidin | Ab + 1 μg/ml sulfo-streptavidin | 25 ng sulfo-streptavidin |
| MSD: R32AP | 3.3 uM (500 μg/ml) sulfo- | 20 nM (3 μg/ml) or 10 nM (1.5 μg/ml) | 75 ng or 37.5 ng sulfo-tagged |
| | tagged anti phosphotyrosine Ab | sulfo-tagged anti phosphotyrosine Ab | anti phosphotyrosine Ab |
| Millipore: | 1 mg/ml 4G10 + 500 μg/ml | 2 μg/ml of each Ab | 50 ng of each Ab |
| 16-103 | SULFO-TAG ® streptavidin | | |

On day 1, two standard (or highbound) MSD 96-well plates (e.g., (MSD-L11XA-3; Meso Scale Discovery; Gaithersburg, Md.) are solution coated with 30 μl of varying amounts of FLT3 capture antibody. One plate is used for total FLT3 detection and the other plate is used for pFLT3 detection. The plates are incubated overnight at 4° C. MV4:11 cells are serum starved overnight in 0.5% FBS.

On day 2, cell lysates are prepared. Briefly, 400,000 serum starved MV4:11 cells are plated per well into two 96-well tissue culture plate in a volume of 100 μl. Other cell concentrations are also tested. To recover the total FLT3 protein, the cells are lysed in 70 μl of a 1x cell lysis buffer (Cat No. 9803 (10×), Cell Signaling Technology, Inc.; Danvers, Mass.), which includes a protease/phosphatase inhibitor cocktail (phenylmethanesulfonyl fluoride (PMSF) used at 1×; Cat. No. P7626; and Sigma Cat. No. 11873580001, Roche, used at 1×) (“cell lysis buffer”) 30 minutes at 4° C. The lysates are then cleared by centrifugation for 15 minutes. The cleared lysates from the two plates are pooled. Seventy microliters of the cleared lysate is added to one of the two anti-FLT3 antibody-coated MSD plates for pFLT3 detection, and 50 μl of the cleared lysate if added to the second anti-FLT3 antibody-coated MSD plate for total FLT3 detection. The two anti-FLT3 antibody-coated MSD plates are blocked with a 3% blocker A solution (Cat No. R93BA-4; Meso Scale Discovery; Gaithersburg, Md.) (“blocking buffer”) solution for 1-2 hours at room temperature prior to lysate addition. Plates are incubated with lysate for 3 hours on a shaker at room temperature. The MSD plates are then washed with wash buffer (50 mM Tris, pH 7.4; 150 mM NaCl; 0.02% Tween) (also available as Cat. No. R61TX-1; Meso Scale Discovery; Gaithersburg, Md.) (“wash buffer”) 3 times using the plate washer.

To detect pFLT3, mixtures are prepared as discussed above, for example, of 2 μg/ml biotinylated 4G10 antibody (Millipore/Upstate 16-103) and 1 μg/ml SULFO-TAG™-labeled streptavidin (Cat No. R32AD; Meso Scale Discovery; Gaithersburg, Md.) in 1% blocking buffer. Twenty-five microliters of this mixture is added to the wells of the pFLT3 plate. To detect total FLT3 (for normalization of the pFLT3 signal), for example, a mixture is prepared of 0.2 μg/ml biotinylated total FLT3 antibody (DYC-912-part #841673 (same as (Cat No. BAF812; R&D Systems, Inc.; Minneapolis, Minn.)) and 1 μg/ml SULFO-TAG™-labeled streptavidin (Cat No. R32AD; Meso Scale Discovery; Gaithersburg, Md.). Twenty-five microliters of this mixture is added to the wells of the total FLT3 plate.

Detection antibodies are incubated for 1 hour on a shaker at room temperature, then the plates are washed three times and 150 μl of the read buffer solution (Cat No. R92TC-1; Meso Scale Discovery; Gaithersburg, Md.) (“read buffer”) is added. The plates are then read on a MA6000 MSD plate reader (Meso Scale Discovery; Gaithersburg, Md.).

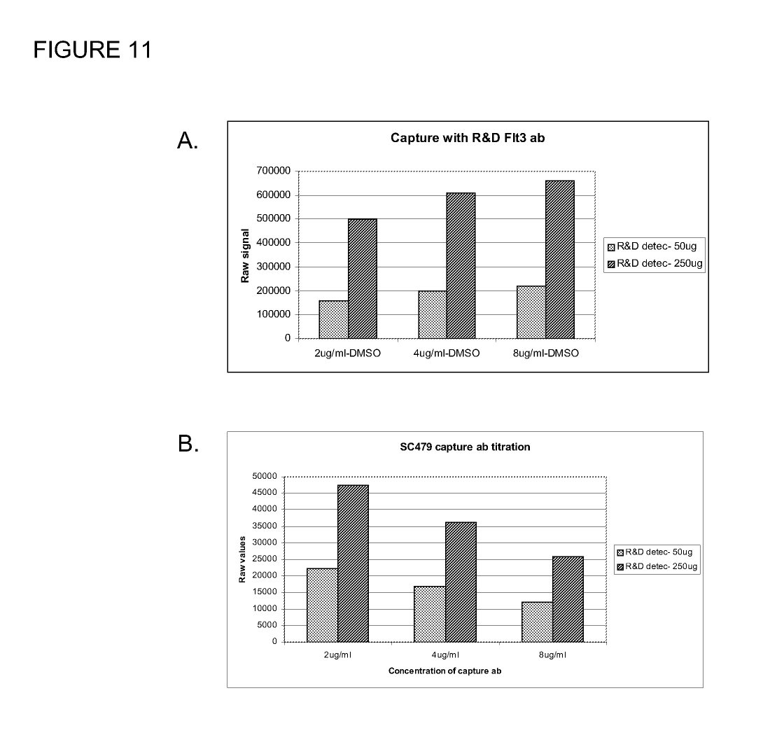

FIG. 11 shows a comparison of MSD ELISA signals obtained from total FLT3 detection using two different first antibodies: mouse anti-human FLT3 IgG mAb (Cat. No. MAB8121; R&D Systems, Inc.; Minneapolis, Minn.) and a rabbit anti-human FLT3 IgG pAb (Cat No. SC-479; Santa Cruz Biotechnology, Inc.; Santa Cruz, Calif.) using R&D detection antibodies BAF812 in both cases. The MAB8121 antibody produces better detection signals than the SC-479 antibody.

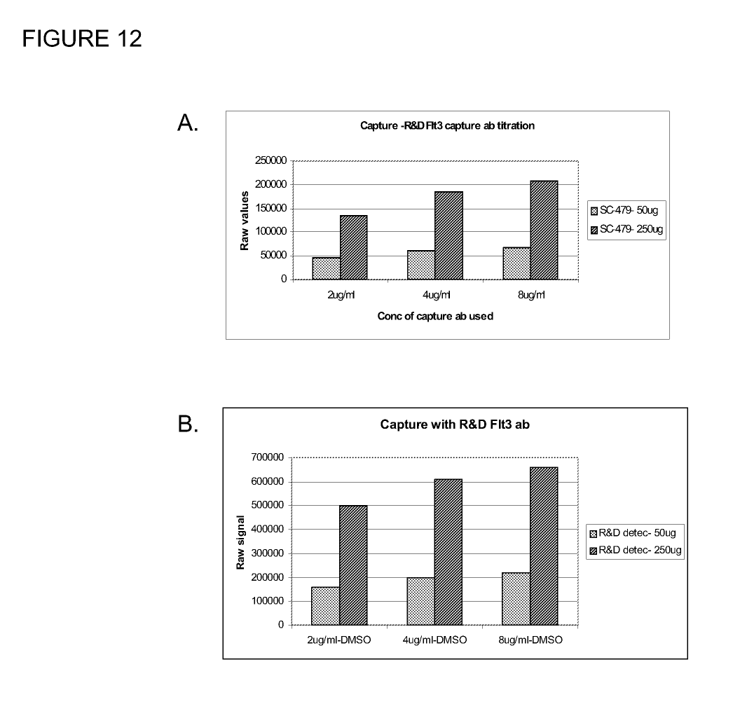

FIG. 12 shows a comparison of MSD ELISA signals obtained from total FLT3 detection using two different second antibodies: rabbit anti-human FLT3 IgG pAb (Cat No. SC-479; Santa Cruz Biotechnology, Inc.; Santa Cruz, Calif.) plus SULFO-TAG™-labeled goat anti-rabbit Ab (Cat No. R32AB; Meso Scale Discovery; Gaithersburg, Md.); and biotinylated goat anti-human FLT3 IgG Ab (Cat No. BAF812; R&D Systems, Inc.; Minneapolis, Minn.) plus SULFO-TAG™-labeled streptavidin (Cat No. R32AD; Meso Scale Discovery; Gaithersburg, Md.), using R&D antibody MAB8121 as the total capture antibody, in both cases.

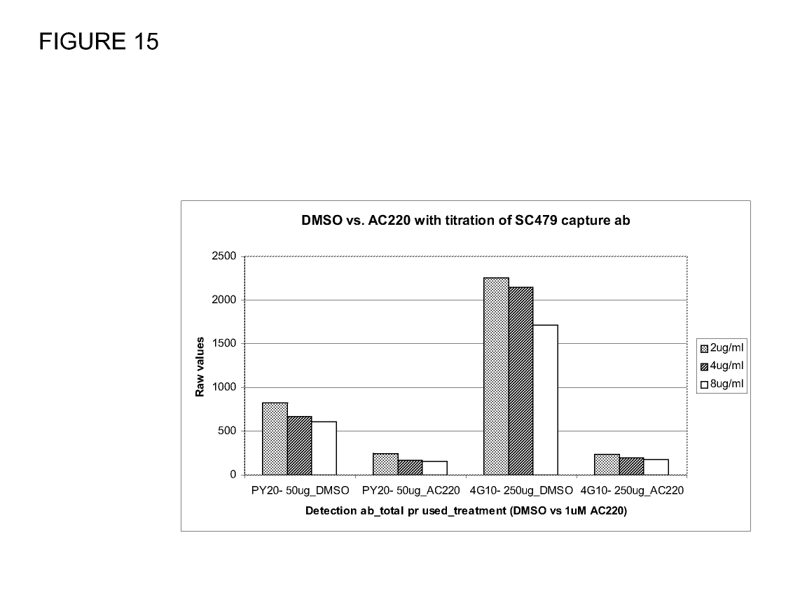

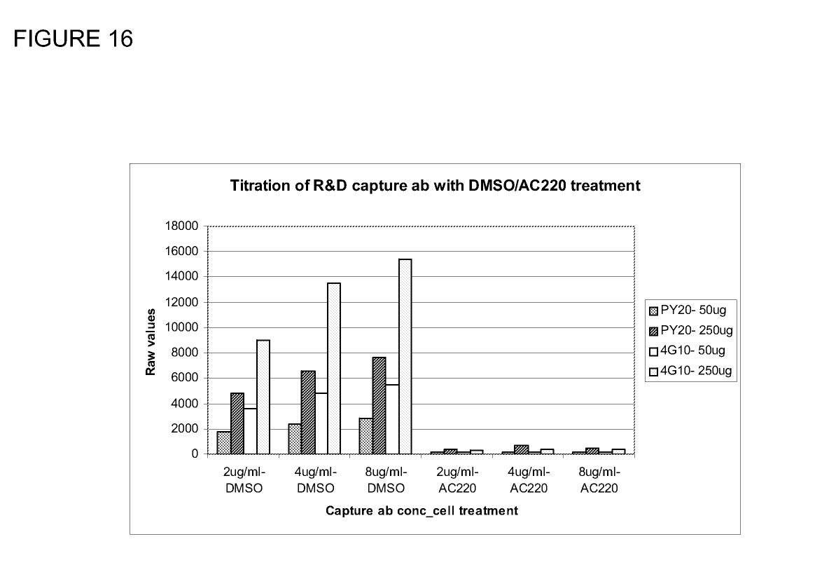

FIGS. 15 and 16 show the MSD signals generated by MV4:11 samples treated with AC220 (see Example 2, below) where there is inhibition of FLT3 phosphorylation by AC220, and MSD signals generated by untreated MV4:11 samples where there is no FLT3 inhibition, in plates coated with SC-479 or MAB8121, respectively, at varying antibody concentrations.

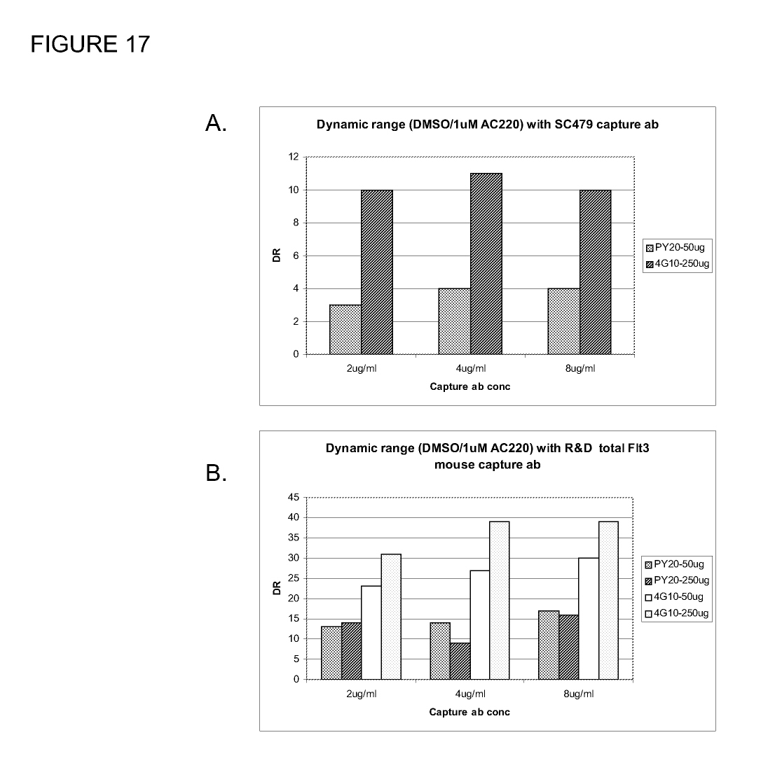

The dynamic ranges for pFLT3 using different capture and detection antibody combinations and concentrations are illustrated in Tables 3 and 4 and FIG. 17. Dynamic range (DR)=pFLT3 in untreated MV4:11 cells/pFLT3 in MV4:11 cells treated with highest concentration of AC220.

TABLE 3
Dynamic range for SC479 capture antibody
and PY20 and 4G10 detection antibodies

| | Capture - SC479 | | |
|---|---|---|---|
| Detection | 2 ug/ml | 4 ug/ml | 8 ug/ml |
| PY20- 50ug_DMSO | 819 | 669 | 607 |
| PY20- 50ug_AC220 | 242 | 169 | 152 |
| DR | 3 | 4 | 4 |
| 4G10- 250ug_DMSO | 2257 | 2150 | 1716 |
| 4G10- 250ug_AC220 | 236 | 196 | 175 |
| DR | 10 | 11 | 10 |

TABLE 4
Dynamic range for R&D capture antibody
and PY20 and 4G10 detection antibodies

| | R&D capture | | |
|---|---|---|---|
| Detection | 2 ug/ml | 4 ug/ml | 8 ug/ml |
| PY20- 50ug_DMSO | 1766 | 2362 | 2803 |
| PY20- 50ug_AC220 | 136 | 164 | 168 |
| DR | 13 | 14 | 17 |
| PY20- 250ug_DMSO | 4791 | 6522 | 7637 |
| PY20- 250ug_AC220 | 346 | 719 | 477 |
| DR | 14 | 9 | 16 |
| 4G10- 50ug_DMSO | 3605 | 4769 | 5499 |
| 4G10- 50ug_AC220 | 158 | 178 | 185 |
| DR | 23 | 27 | 30 |
| 4G10- 250ug_DMSO | 8972 | 13520 | 15409 |
| 4G10- 250ug_AC220 | 289 | 345 | 397 |
| DR | 31 | 39 | 39 |

Table 3 shows that the dynamic range (DR) obtained from the SC479 capture antibody/PY20 second antibody of 3 or 4 is low, while the dynamic range obtained from the SC479 capture antibody/4G10 second antibody of 10 or 11 is good, although a significant amount of total protein (250 ug) was required. However, Table 4 shows that the dynamic range of 39 obtained from the R&D MAB8121 antibody/4G10 second antibody pairing clearly made this combination preferable to the R&D MAB8121 antibody/PY20 second antibody pairing, and even at the lower sample protein content of 50 ug, the dynamic range of 27 or 30 was still very good. Because the signal appeared to reach a plateau at 4 ug/mL of capture antibody, this capture antibody concentration was chosen in the preferred antibody combination. A preferred antibody combination was therefore arrived at based on the strongest MSD ELISA signal and broadest dynamic range that could be obtained with the least amount of protein sample input.

A preferred antibody combination for the total FLT3 ELISA is as follows: Capture: Mouse anti-human FLT3 IgG mAb (Cat. No. MAB8121; R&D Systems, Inc.; Minneapolis, Minn.) at 120 ng per well. Detection (mixture in 25 μl total volume per well): Biotinylated goat anti-human FLT3 IgG Ab (Cat No. BAF812; R&D Systems, Inc.; Minneapolis, Minn.) at 5 ng per well (0.2 μg/ml in 25 μl volume) plus SULFO-TAG™-labeled streptavidin (Cat No. R32AD; Meso Scale Discovery; Gaithersburg, Md.) at 25 ng per well (1 μg/ml in 25 μl volume). A preferred antibody combination for the pFLT3 ELISA is as follows: Capture: Mouse anti-human FLT3 IgG mAb (Cat. No. MAB8121; R&D Systems, Inc.; Minneapolis, Minn.) at 120 ng per well. Detection: Biotinylated mouse anti-phospho-tyrosine (4G10®) mAb (Cat No. 16-103; Millipore Corp.; Billerica, Mass.) at 50 ng per well plus SULFO-TAG™-labeled streptavidin (Cat No. R32AD; Meso Scale Discovery; Gaithersburg, Md.) at 50 ng per well.

### Example 2

### In Vivo FLT3 MSD ELISA for Testing pFLT3

AC220 is a second generation class III receptor tyrosine kinase inhibitor with potent in vitro and in vivo activity in FLT3-dependent tumors. It is highly selective for wild-type and mutant FLT3 and several class III receptor tyrosine kinases including KIT, CSF1R, RET and PDGFR. AC220 is currently in a first in human phase I study for relapsed and refractory patients unselected for FLT3 mutations. The study has a standard 3+3 dose escalation design with 50% dose increments. AC220 was administered at a starting dose of 12 mg once daily as an oral solution for 14 days followed by a 14 day rest period.

Whole blood samples from 51 patients who received AC220 in the phase I study were collected and tested for pFLT3 inhibition. The blood samples were drawn from the patients at various time points during the course of AC220 therapy, including a sample drawn before AC220 administration (predose) and samples drawn at 2, 4, 6, 9 and 24 hours after the initial dose of AC220, and also drawn at 8 days and 15 days after the initial dose. The blood samples were collected in heparinized tubes, gently mixed and then lysed with a 1× cell lysis buffer (20 mM Tris, 137 nM NaCl, 10% glycerol, 1% NP-40, 0.1% SDS, 2 mM EDTA) supplemented with a protease/phosphatase inhibitor cocktail (PMSF used at 1×; Cat. No. P7626; and Sigma Cat. No. 11873580001, Roche, used at 1×). The samples were then flash frozen in −70° C. A small number of patient samples were frozen unlysed and lysed afterwards.

On day 1, a standard MSD 96-well plate (MSD-L11XA-3; Meso Scale Discovery; Gaithersburg, Md.) is coated with 30 μl of 4 μg/ml mouse anti-human FLT3 IgG mAb (Cat. No. MAB8121; R&D Systems, Inc.; Minneapolis, Minn.) and incubated overnight at 4° C. The top half (rows A-H) of the plate is used to detect pFLT3 and the bottom half (rows E-H) of the plate was used to detect total FLT3.

On day 2, the FLT3 antibody-coated 96-well plates are blocked with 150 μl of 3% blocker A solution (Cat No. R93BA-4; Meso Scale Discovery; Gaithersburg, Md.) for 1-2 hours at room temperature prior to sample addition. During the blocking step, blood samples are prepared. A 1.5 ml aliquot of lysed and frozen blood is thawed at 4° C. and incubated for 30-60 minutes on a rocker. The lysed blood is cleared by centrifugation at maximum speed for 10 minutes at 4° C. The cleared blood is then transferred to fresh tubes. The blocked 96-well plates are then washed, and 250 μl of cleared blood is transferred to duplicate wells on two rows of the plate (one row for pFLT3 detection and a second row for total FLT3 detection). A total of 1 ml of blood was required to detect the pFLT3 and total FLT3 in duplicate. The plates are then incubated for 18-20 hrs. (overnight) on a shaker at 4° C.

The total FLT3 values serve, for example, as an assay control to verify that the FLT3 protein was indeed captured by the capture antibody (as confirmed by the detection of the second anti-total FLT3 antibody binding to the captured FLT3). Secondly, as explained below, the tFLT3 values can also be used to normalize the pFLT3 values.

The following day, the blood is removed from the MSD plates and the plates washed 3 times. To detect pFLT3, a cocktail of 2 μg/ml biotinylated mouse anti-phospho-tyrosine (4G10®) mAb (Cat No. 16-103; Millipore Corp.; Billerica, Mass.) and 2 μg/ml SULFO-TAG™-labeled streptavidin (Cat No. R32AD; Meso Scale Discovery; Gaithersburg, Md.) is prepared in 1% blocking buffer. Twenty-five microliter of this antibody mixture was added to the first 4 rows (A-D) of the MSD plate. To detect total FLT3 (for normalization of the pFLT3 signal), for example, a mixture is prepared of 0.2 μg/ml biotinylated total FLT3 antibody (DYC-912-part #841673 (same as (Cat No. BAF812; R&D Systems, Inc.; Minneapolis, Minn.)) and 1 μg/ml SULFO-TAG™-labeled streptavidin (Cat No. R32AD; Meso Scale Discovery; Gaithersburg, Md.). Twenty-five microliters of this antibody mixture is added to the last 4 rows (E-H) of the MSD plate. The plate is then incubated for 1 hour on a shaker at room temperature. The plates are then washed 3 times in wash buffer (50 mM Tris, pH 7.4; 150 mM NaCl; 0.02% Tween) (also available as Cat. No. R61TX-1; Meso Scale Discovery; Gaithersburg, Md.). 150 μl of read buffer solution (Cat No. R92TC-1; Meso Scale Discovery; Gaithersburg, Md.) is added and the plates are read on a MA6000 MSD plate reader (Meso Scale Discovery; Gaithersburg, Md.).

Results: The range of signal for pFLT3 patients ranged from 120 (obtained from a low allelic ratio ITD patient having less than 2% ITD as well as from wild type patients) to as high as 6000-8000 (obtained mostly from patients with high allelic ratio ITD). The following is a discussion on the pFLT3 and tFLT3 levels measured for four select patient samples using methods of the invention.

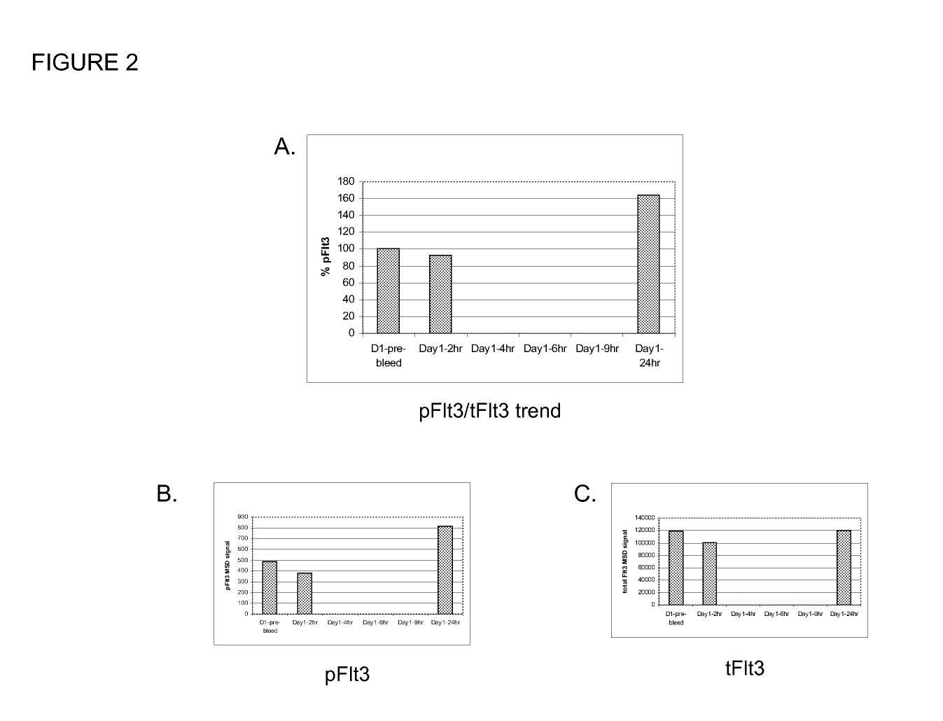

Among the ITD patients tested, patient A was found to have the lowest MSD signal in the pFLT3 ELISA (a signal of about 500, see FIG. 2B). PhosphoFLT3 levels were normalized with tFLT3 levels and reported as a ratio of pFLT3/tFLT3 to ensure that reported pFLT3 trends were not distorted by tFLT3 trends. The pFLT3 trends for all patient samples at the various post dose timepoints were tracked in terms of percentages calculated by dividing the ratio of pFLT3/tFLT3 observed for a particular post dose time point by the initial ratio of pFLT3/tFLT3 measured at predose, which was arbitrarily set at 100%. Although genotyped as 100% ITD, Patient A showed an uncharacteristically low signal for pFLT3. This patient is the only ITD patient in the sampling of patients who did not show a marked decrease in pFLT3 levels upon treatment with AC220, although treatment appeared to reduce bone marrow blast percentage.

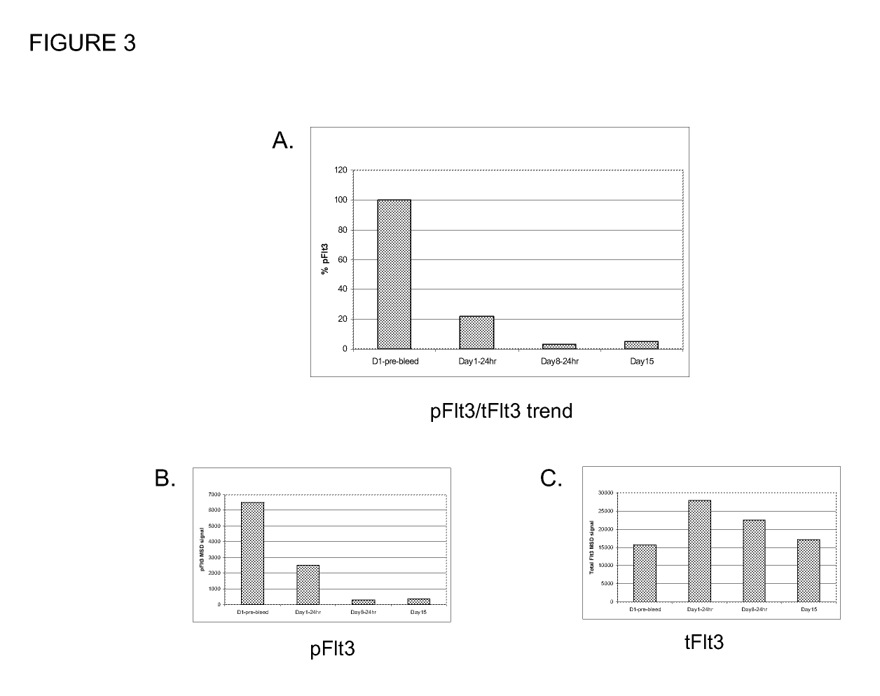

Patient B is a 100% ITD patient that exhibits a pFLT3 profile that is expected of a 100% ITD patient. The patient shows a strong MSD signal of 6500 (see FIG. 3B), which is uncharacteristically strong for even an ITD patient. This patient shows a dramatic decrease in pFLT3 levels in 24 hours (despite the upward shift in total FLT3 (tFLT3) as shown in FIG. 3C). Patient B shows a striking inhibition of pFLT3 that is sustained even up to Day 15, (FIG. 3C) and the ELISA pFLT3 data is consistent with the clinical observation of 73% peripheral blast reduction for this patient.

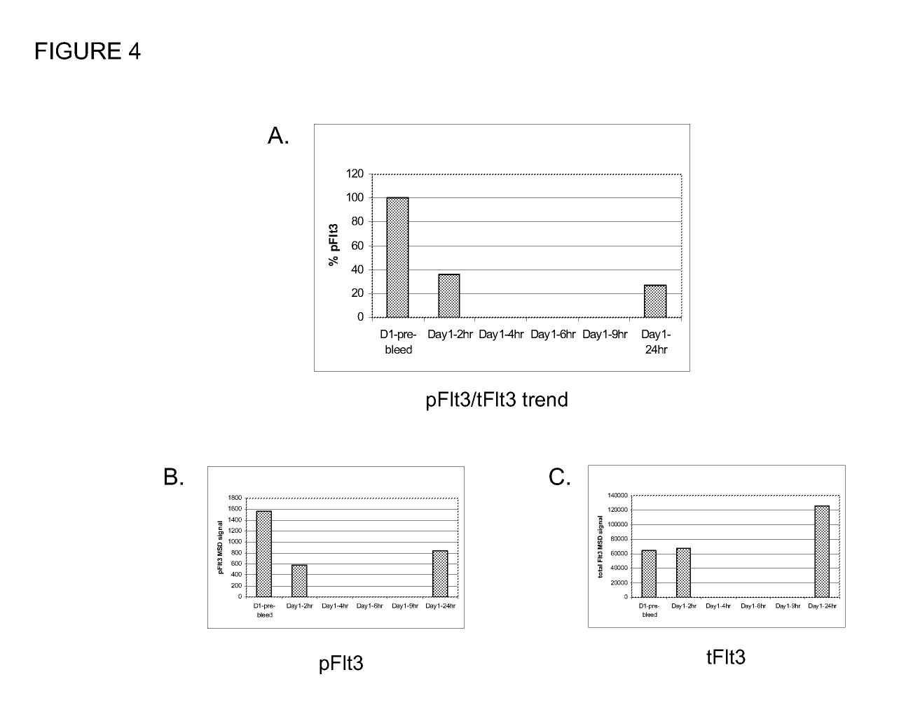

Patient C, of undetermined genotype, shows a strong prebleed signal of about 1500 and shows clear reduction in pFLT3 levels at the 2 hour timepoint, with pFLT3 inhibition sustained at 24 hours (FIG. 4). The pFLT3 data is consistent with the clinical observation of this patient, exhibited 100% peripheral blast reduction and met the protocol defined response of PR.

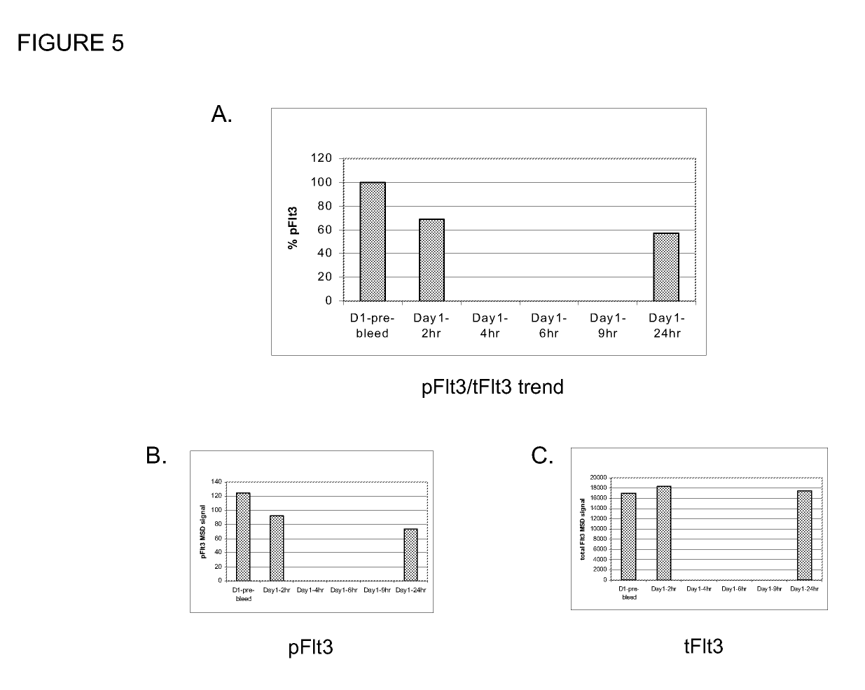

Patient D is a wild type patient whose plasma sample produced a weak pre-bleed pFLT3 signal of about 125 (FIG. 5B). Despite the weak starting signal, the assay was sensitive enough to detect a downward shift in pFLT3 levels at 2 hours (signal strength went down to about 90) and was able to show a greater inhibition at 24 hours (signal strength of about 75). The pFLT3 inhibition observed for this patient from the ELISA appears to be consistent with the fact that this patient also exhibited a protocol defined response of CRp/PR (complete remission, with incomplete platelet recovery).

### Example 3

### FLT3 MSD ELISA for Testing pFLT3 Inhibition in MV4:11 Cell Lysates

MV4-11 is a well-characterized FLT3-dependent human cell line that contains an internal tandem duplication (ITD) found in a subset of patients with acute myeloid leukemia. The MV4:11 cells express constitutively active FLT3 receptors (Yee et al. Blood 2002, 100(8), 2941-2949) and is therefore a good source of pFLT3.

On day 1, two standard MSD 96-well plates (MSD-L11XA-3; Meso Scale Discovery; Gaithersburg, Md.) are coated with 30 μl of 4 μg/ml mouse anti-human FLT3 IgG mAb (Cat. No. MAB8121; R&D Systems, Inc.; Minneapolis, Minn.). One plate is used for pFLT3 detection and the other for total FLT3 detection. The plates are incubated overnight at 4° C. MV4:11 cells were serum starved overnight in 0.5% FBS.

On day 2, cell lysates are prepared. Briefly, 400,000 serum starved MV4:11 cells are plated per well into two 96-well tissue culture plate in a volume of 100 μl. Compounds to be tested are prepared as a 2× stock in 0.5% FBS media in a total volume of 250 μl each. 100 μl of 2× stock test compound is then added to the 100 μl of cells in the 96-well plate. A 9 point, 3× dose titration is used for each of the inhibitors. Duplicate 96-well plates with cells are incubated with the test compound for 2 hours at 37° C. To recover the total FLT3 protein, the cells are lysed in 70 μl of a 1× cell lysis buffer (Cat No. 9803 (10×), Cell Signaling Technology, Inc.; Danvers, Mass.), which includes a protease/phosphatase inhibitor cocktail (PMSF used at 1×; Cat. No. P7626; and Sigma Cat. No. 11873580001, Roche, used at 1×) for 30 minutes at 4° C. The lysates are then cleared by centrifugation for 15 minutes. The cleared lysates from the two plates are pooled. Seventy microliters of the cleared lysate is added to one of the two anti-FLT3 antibody-coated MSD plates for pFLT3 detection, and 40 μl of the cleared lysate if added to the second anti-FLT3 antibody-coated MSD plate for total FLT3 detection. The two anti-FLT3 antibody-coated MSD plates are blocked with a 3% blocker A solution (Cat No. R93BA-4; Meso Scale Discovery; Gaithersburg, Md.) for 1-2 hours at room temperature prior to lysate addition. Plates are incubated with lysate for 3 hours on a shaker at room temperature.

The MSD plates are then washed with wash buffer (50 mM Tris, pH 7.4; 150 mM NaCl; 0.02% Tween) (also available as Cat. No. R61TX-1; Meso Scale Discovery; Gaithersburg, Md.) three times using the plate washer. To detect pFLT3, a mixture is prepared of 2 μg/ml biotinylated mouse anti-phospho-tyrosine (4G10®) mAb (Cat No. 16-103; Millipore Corp.; Billerica, Mass.) and 1 μg/ml SULFO-TAG™-labeled streptavidin (Cat No. R32AD; Meso Scale Discovery; Gaithersburg, Md.) in 1% blocking buffer. Twenty-five microliters of this mixture is added to the wells of the pFLT3 plate. To detect total FLT3 (for normalization of the pFLT3 signal), a cocktail of 0.2 μg/ml biotinylated mouse anti-phospho-tyrosine (4G10®) mAb (Cat No. 16-103; Millipore Corp.; Billerica, Mass.) and 1 μg/ml SULFO-TAG™-labeled streptavidin (Cat No. R32AD; Meso Scale Discovery; Gaithersburg, Md.) is prepared in 1% blocking buffer. Twenty-five microliters of this antibody mixture is added to the total FLT3 plate. The plates are then incubated for 1 hour on a shaker at room temperature. The plates are then washed 3 times. 150 μl of the read buffer solution (Cat No. R92TC-1; Meso Scale Discovery; Gaithersburg, Md.) is added, and the plates are read on a MA6000 MSD plate reader (Meso Scale Discovery; Gaithersburg, Md.).

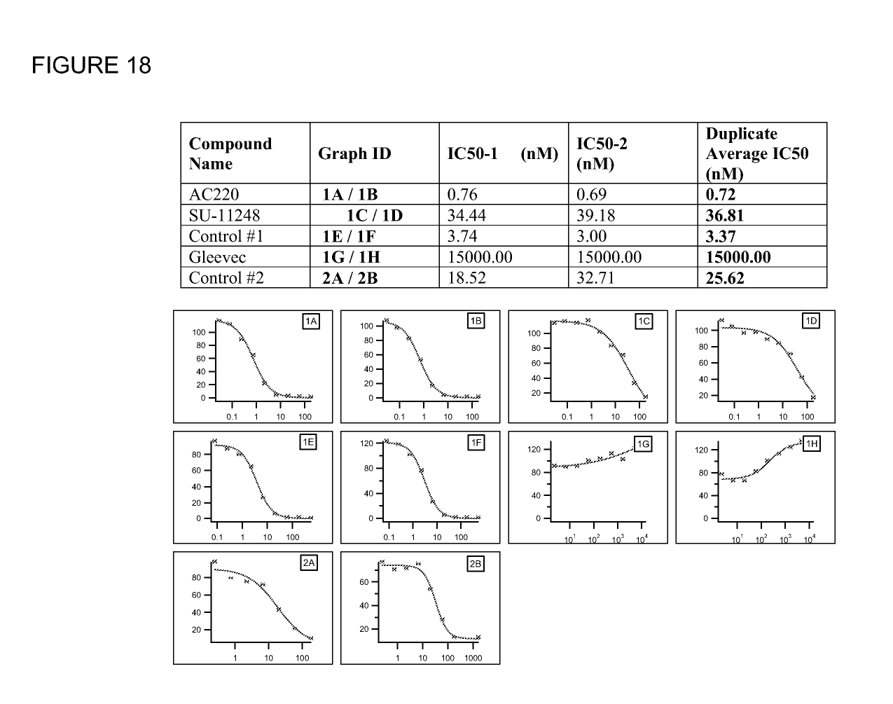

The inhibition of phospho FLT3 (IC₅₀) is determined from a dose-response curve that shows the inhibition of phosphorylation of FLT3 in the presence of FLT3 inhibitors (compounds AC010220 (AC220), AB000709, AC010385, AB200530) at various concentrations compared to a negative control (AB00022 (GLEEVEC™)) (FIG. 18). The signals of pFLT3 were normalized to total FLT3 to account for the difference in cell numbers in different wells. Due to the requirement of large samples and low signal of prior IP/Western methods, the IC₅₀ values of compounds AB000709 (sunitinib), AC010385, AB200530 were not able to be determined previously but were successfully determined herein using methods of the invention. The ICₛₒ value for compound AC010220 had previously been determined to be about 0.6-0.8 measured by IP/Western, which is similar to the 0.76 value measured in the present assay. The dynamic range (signal to noise) in this assay was also shown to be 82.

These results indicate that IC₅₀ of FLT3 inhibition by FLT3 inhibitors can be measured by the MSD ELISA assays in cells expressing FLT3.

### Example 4

### Determination of MSD ELISA Sensitivity with Blood Samples Compared to IP/Western

A titration of MV4:11 cells in blood was tested by IP/Western blot and by MSD ELISA to compare the sensitivity of pFLT3 detection by the two methods.

For the IP/Westerns, cell lysate from MV4:11 cells were spiked into normal blood. On day one, the serum starved MV4:11 4×10⁷ cells were counted, washed with PBS and placed in low serum (Iscoves 0.5% FBS).

On day two, 1 ml of blood plus 0.5 ml of MV4:11 cells was subjected to 3 ml of lysis buffer (1× cell lysis buffer (20 mM Tris, 137 nM NaCl, 10% glycerol, 1% NP-40, 0.1% SDS, 2 mM EDTA) supplemented with protease and phosphatase inhibitors (Cat#11873580001, Roche used at 1:20 dilution and Cat#524625 EMD_Calbiochem at a 1:100 dilution) rotated for 30 minutes at 4° C., and then spun down for 15 minutes to extract the lysate. One milliliter of this sample was then divided equally into four 250 μl samples and run in pFLT3 or tFLT3 MSD ELISAs (in duplicate) as described above. The remaining 3.5 ml was used for the IP/Western (i.e., 14-fold more sample run in the IP/Western). Nine samples were generated from a two-fold serial dilution of MV4:11 cells starting at a 2.6 million cells down to 10,000 cells. 4 μg of FLT3 antibody (Cat No. SC479 Santa Cruz Biotechnology, Inc.) was added to the cleared lysate from each sample to immunoprecipitate the FLT3 protein and incubated overnight at 4° C. to immunoprecipitate phosphorylated FLT3 protein.

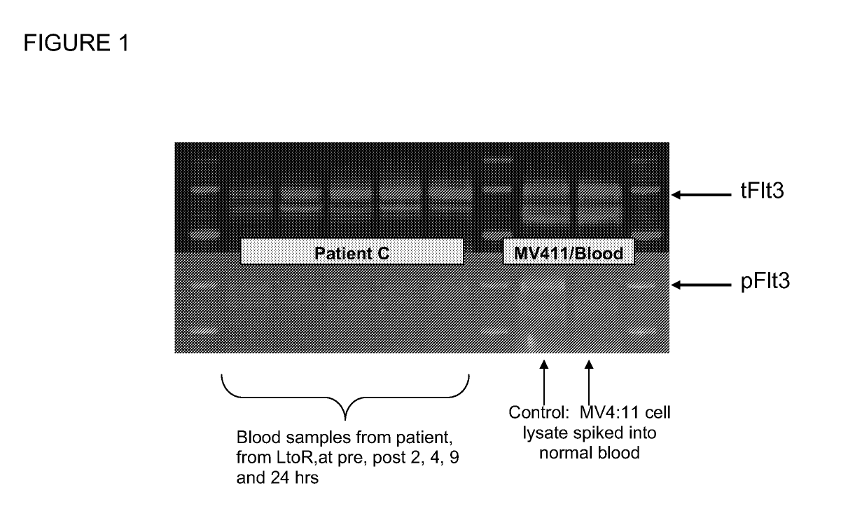

On day three, protein A-agarose beads (50 μL) were added to all the samples, and allowed to incubate at 4° C. for two hours with rotation. The beads were washed three times with sample buffer, boiled, spun and then loaded on a 3-8% SDS-PAGE gel and subjected to electrophoresis. The gel is then transferred to a nictrocellulose membrane and blocked for 1 hr with Li-COR blocking buffer (Cat# 927-40000 Li-COR Biosciences). The blot is then probed with a mixture of primary antibodies against total Flt3 (Cat No. SC479 Santa Cruz Biotechnology, Inc.) and an antibody against phosphotyrosines to detect phospho Flt3 (4G10R) (Cat No. 16-316; Millipore Corp.; Billerica, Mass.) overnight at 4° C. at a dilution of 1:500 in blocking buffer. The next day the blots are washed and incubated with secondary antibodies at a dilution of 1:15000. The secondary antibody mix contains two antibodies: a goat anti-rabbit secondary antibody conjugated to the IR-680 dye-red (Cat# 926-32221 Li-COR Biosciences) to detect the rabbit Flt3 total antibody and a goat anti-mouse secondary antibody conjugated to the IR-800 dye-green (Cat# 926-32210 Li-COR Biosciences) to detect the mouse 4G10 antibody. The blots were then washed, imaged and the bands quantified using the Odyssey Infrared Imaging System (Li-COR Biosciences). FIG. 1 shows the pFLT3 and tFLT3 IP/Western blot result for Patient A. Compared to the MSD data for the same patient in FIG. 2 which shows an increase in pFLT3 at 24 hours post dose compared to predose (pre-bleed), it is harder to detect this trend in pFLT3 levels using IP/Western.

The MSD assays are conducted essentially as provided in the Examples above. Two hundred and fifty microliters of cleared blood plus MV4:11 lysates prepared as described earlier are added to the anti-FLT3 antibody-coated plates, for the detection of total FLT3 and pFLT3. The two anti-FLT3 antibody-coated MSD plates are blocked with a 3% blocker A solution (Cat No. R93BA-4; Meso Scale Discovery; Gaithersburg, Md.) for 1-2 hours at room temperature prior to lysate addition. Plates are incubated overnight on a shaker at 4° C.

On the following day, the MSD plates are washed with wash buffer (50 mM Tris, pH 7.4; 150 mM NaCl; 0.02% Tween) (also available as Cat. No. R61TX-1; Meso Scale Discovery; Gaithersburg, Md.) three times using the plate washer. To detect pFLT3, a mixture is prepared of 2 μg/ml biotinylated mouse anti-phospho-tyrosine (4G10R) mAb (Cat No. 16-103; Millipore Corp.; Billerica, Mass.) and 2 μg/ml SULFO-TAG™-labeled streptavidin (Cat No. R32AD; Meso Scale Discovery; Gaithersburg, Md.) in 1% blocking buffer. Twenty-five microliters of this mixture is added to the wells of the pFLT3 plate. To detect total FLT3 (for normalization of the pFLT3 signal), a cocktail of 0.2 μg/ml biotinylated mouse anti-phospho-tyrosine (4G10®) mAb (Cat No. 16-103; Millipore Corp.; Billerica, Mass.) and 1 μg/ml SULFO-TAG™-labeled streptavidin (Cat No. R32AD; Meso Scale Discovery; Gaithersburg, Md.) is prepared in 1% blocking buffer. Twenty-five microliters of this antibody mixture is added to the wells of the total MSD plate. The plate is then incubated for 1 hour on a shaker at room temperature. The plates are then washed 3 times. 150 μl of the read buffer solution (Cat No. R92TC-1; Meso Scale Discovery; Gaithersburg, Md.) is added, and the plates are read on a MA6000 MSD plate reader (Meso Scale Discovery; Gaithersburg, Md.).

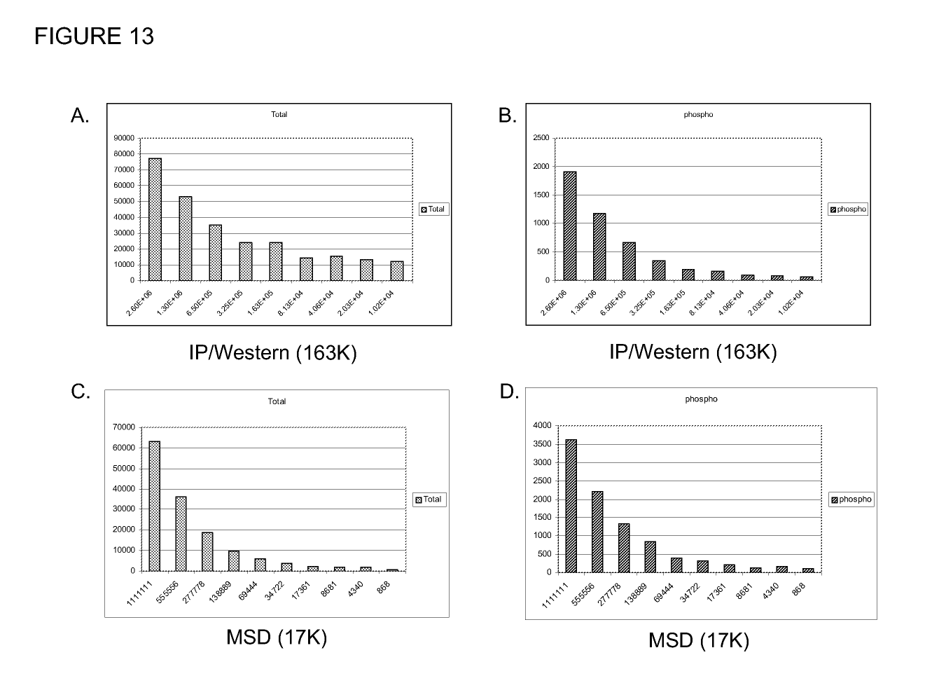

A comparison of detection sensitivity produced by IP/Western and MSD ELISA in a different experiment is shown in FIG. 13. As shown in FIG. 13, a titration of pFLT3 MSD ELISA signal was achieved from 1.11×10⁶ cells down to approximately 17,000 cells (FIG. 13D). In contrast, the linearity of the IP Western signal was observed only down to 163,000 cells (FIG. 13C). A comparison of the limit of detection of these two assays as presented in FIG. 13 demonstrates that the MSD ELISA of the invention is at least five to ten times more sensitive than the IP/Western in detecting total FLT3 and pFLT3 from a blood sample. When taking into account the 14-fold less protein used for the MSD ELISA, the actual sensitivity of this method is at least 60- to 140-fold greater than the IP/Western.

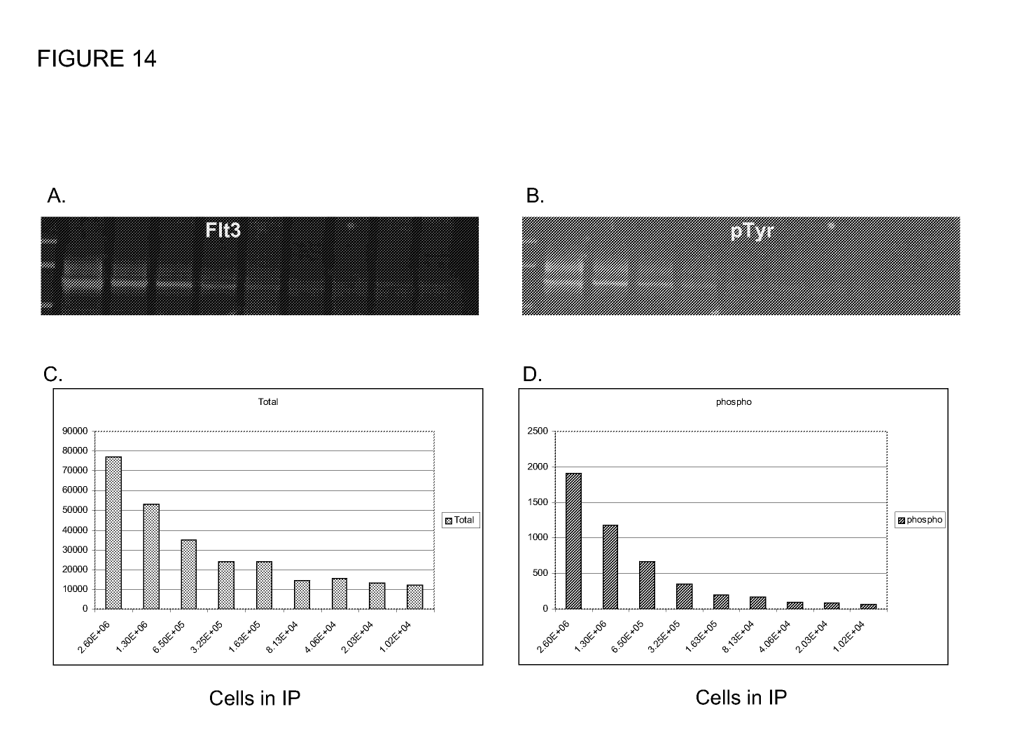

The corresponding western blots of total FLT3 and pFLT3 are shown in FIG. 14. FIGS. 14A and 14B show the Flt3 and pFlt3 bands from the Western blot and FIGS. 14C and 14D represents the signals obtained (y-axis) for each titration point containing the number of cells indicated at the x-axis.

### Example 5

### FLT3 MSD ELISA for Testing pFLT3 Inhibition in Cell Lines Expressing Wild-Type or Mutant Flt3

The ELISA assays described herein are modified for testing pFLT3 inhibition in cell lines that express wild-type or mutant FLT3. The procedure is optimized essentially as described in Example 1.

On day 1, standard MSD 96-well plates (MSD-L11XA-3; Meso Scale Discovery; Gaithersburg, Md.) are solution coated with 30 μL of 4 μg/mL of mouse anti-human FLT3 IgG mAb (Cat. No. MAB8121; R&D Systems, Inc.; Minneapolis, Minn. or Cat No. SC-479; Santa Cruz Biotechnology, Inc.; Santa Cruz, Calif.). One plate is used for pFLT3 detection and the other for total FLT3 detection. The detection of total FLT3 is for normalization of the pFLT3 signal. The plates are incubated overnight at 4° C. MV4:11 or RS4:11 cells are serum starved overnight in 0.5% FBS. The RS4-11 is a cell line that expresses the wild-type (WT) receptor which is expected to express low levels of pFLT3. For this cell line, FLT3 ligand was used to stimulate pFLT3 in RS4:11 cells.

On day 2, cell lysates are prepared. Briefly, 400,000 serum starved MV4:11 or RS4:11 cells are plated per well in duplicate 96-well tissue culture plates in a volume of 100 μl. FLT3 inhibitors (e.g., AC220 and others) are prepared as a 2× stock in 0.5% FBS media in a total volume of 250 μL each. 100 μL of 2× stock test compound in DMSO or negative control DMSO is then added to the 100 μL of cells in the 96-well plate. A 9 point, 3× dose titration is used for each of the inhibitors. The cells are incubated with the test compound for 2 hours at 37° C. Prior to cell lysis, the wildtype FLT3 in RS4:11 cells were stimulated by the addition of 100 ng/mL FLT3 ligand (R&D Cat No. 308-Fk) for 15 minutes. To recover the total FLT3 protein, the cells are lysed in 70 μL of 1× cell lysis buffer (Cat No. 9803 (10×), Cell Signaling Technology, Inc.; Danvers, Mass.), which includes a protease/phosphatase inhibitor cocktail (PMSF used at 1×; Cat. No. P7626; and Sigma Cat. No. 11873580001, Roche, used at 1×) for 30 minutes at 4° C. The lysates are then cleared by centrifugation for 15 minutes and then pooled. Seventy microliters of the cleared lysate is added to one of the two anti-FLT3 antibody-coated MSD plates for pFLT3 detection, and 40 μl of the cleared lysate if added to the second anti-FLT3 antibody-coated MSD plate for total FLT3 detection. The two anti-FLT3 antibody-coated MSD plates are blocked with a 3% blocker A solution (Cat No. R93BA-4; Meso Scale Discovery; Gaithersburg, Md.) for 1-2 hours at room temperature prior to lysate addition. Plates are incubated with lysate for 3 hours on a shaker at room temperature.

The MSD plates are then washed with wash buffer (50 mM Tris, pH 7.4; 150 mM NaCl; 0.02% Tween) (also available as Cat. No. R61TX-1; Meso Scale Discovery; Gaithersburg, Md.) three times using the plate washer. To detect pFLT3, a mixture is prepared of 2 μg/ml biotinylated mouse anti-phospho-tyrosine (4G10®) mAb (Cat No. 16-103; Millipore Corp.; Billerica, Mass.) and 1 μg/ml SULFO-TAG™-labeled streptavidin (Cat No. R32AD; Meso Scale Discovery; Gaithersburg, Md.) in 1% blocking buffer. Twenty-five microliters of this mixture is added to the wells of the pFLT3 plate. To detect total FLT3 (for normalization of the pFLT3 signal), a cocktail of 0.2 μg/ml biotinylated mouse anti-phospho-tyrosine (4G10®) mAb (Cat No. 16-103; Millipore Corp.; Billerica, Mass.) and 1 μg/ml SULFO-TAG™-labeled streptavidin (Cat No. R32AD; Meso Scale Discovery; Gaithersburg, Md.) is prepared in 1% blocking buffer. Twenty-five microliters of this antibody mixture is added to the wells of the total FLT3 plate.

The plate is then incubated for 1 hour on a shaker at room temperature. The plates are then washed 3 times. 150 μl of the read buffer solution (Cat No. R92TC-1; Meso Scale Discovery; Gaithersburg, Md.) is added, and the plates are read on a MA6000 MSD plate reader (Meso Scale Discovery; Gaithersburg, Md.).

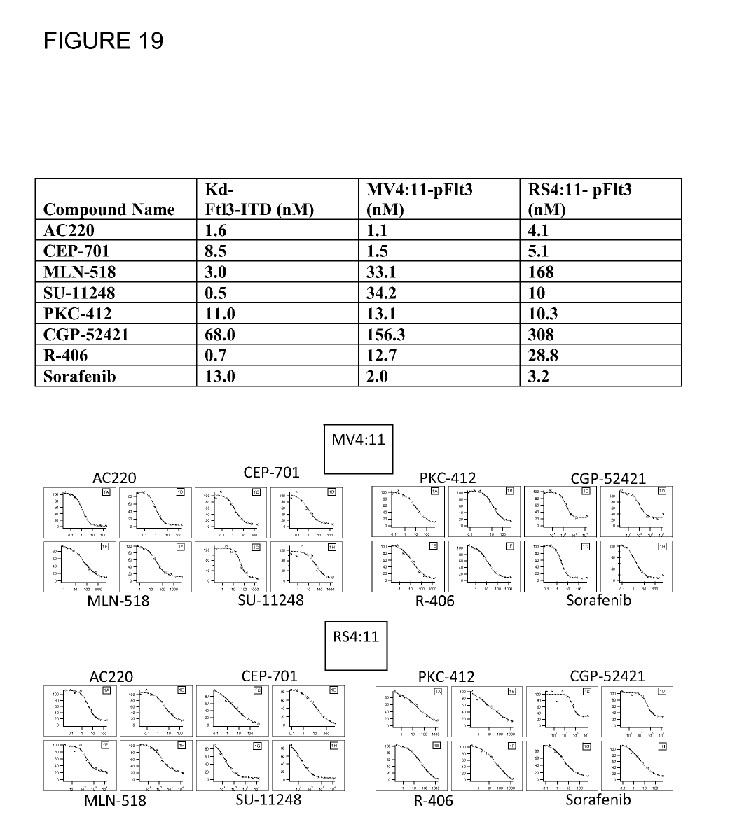

FIG. 19 shows the dose response curves for AC220 and other reference compounds known in the literature to be FLT3 inhibitors, namely, CEP-701, MLN-518, SU-11248, PKC-412, CGP-52421, R-406 and sorafenib obtained from this MSD ELISA protocol. The IC50s for these reference compounds correlate very well with published IC50s and further validates this method of the invention.

### Example 6

### FLT3 MSD ELISA for Testing pFLT3 Inhibition in Mouse Tumor Xenograft

The efficacy of AC220 was determined in a SCID mouse engraftment model where intravenously inoculated FLT3-ITD-dependent MV4:11 cells disseminate to the bone marrow. 5×10⁶ MV4:11 cells were implanted into the flanks of SCID mice. The tumors were allowed to grow to size then the mouse was orally dosed with 10 mg/kg AC220 formulated with 1:1 of PBS and 50% Matrigel/50% High Concentration Matrigel. Tumor samples were collected from untreated control group and from AC220 treated group, taken at 2 hour and 24 hour post dose timepoints. The tumors were weighed then homogenized in lysis buffer (15 mg/mL). The cells were spun down in a cold centrifuge at 3000 rpm for 15 minutes and the clear lysate was collected and tested according to the exemplary protocol in Example 2 (with 250 uL of cleared tumor lysate added to wells on anti-FLT3 coated MSD plates).

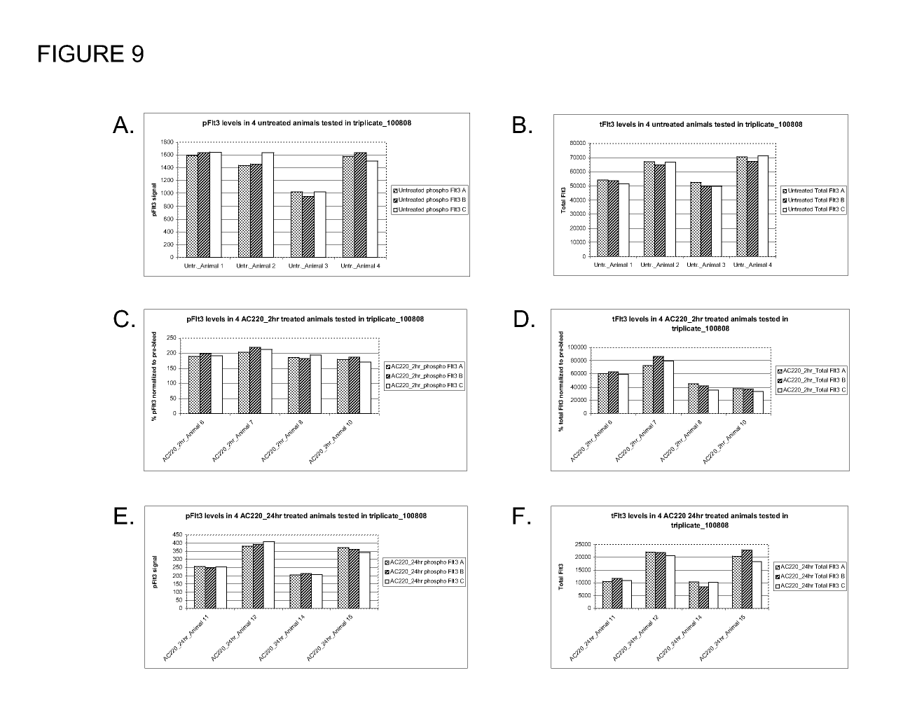

FIG. 9 shows the signal levels obtained from MV4:11 implanted tumor samples. Using methods of the invention, the tumor sample generated a high MSD signal for both pFLT3 and tFLT3, confirming expectations that the tumor cell contains a more concentrated amount of cells expressing FLT3.

### Example 7

### In Vivo FLT3 MSD ELISA for Testing pFLT3 Inhibition in Other Bodily Fluid and Tissue Samples

The protocol(s) described in the Example(s) above may be used in in vivo samples such as bone marrow samples, spinal fluid, cerebral fluid, cytological samples and aspirates. These samples may be used directly without further processing and loaded onto the anti-FLT3 coated MSD ELISA plates.

### Example 8

### Diagnostic Assay for the Status of Wild-Type or FLT3 Mutations in a Patient

The ELISA assays disclosed herein are modified for diagnosing the status of wild-type or activating FLT3 mutations (e.g., FLT3-ITD) in a patient. The blood samples are taken from patients with AML or other FLT3-mediated diseases at various stages of the disease. The blood samples are then snap frozen in liquid nitrogen.

An exemplary protocol for detecting total and phosphorylated FLT3 in blood is described in Example 2. In addition, and exemplary procedure for creating a probability table useful for diagnosing a FLT3 mutation in a patient is provided below.

An analysis of blood samples from 45 patients undergoing AC220, taken before treatment with AC220 and subsequent time points taken during treatment were analyzed. Based on the distribution of the signal and the range of signal observed among these 45 samples, a sample generating a signal less than 500 obtained from the MSD detection method was designated as having a “low” level of pFLT3; a sample generating a signal equal to or greater than 500 and less than 1000 was designated as “medium” level of pFLT3; and a sample generating a signal of 1000 or greater was designated as “high” level of pFLT3. Based on these designations as guidelines, a blood sample tested using the in vivo MSD FLT3 ELISA assays described herein can be determined to express low, medium or high levels of pFLT3 based on the MSD signal generated by that sample.

For a bone marrow sample tested for pFLT3 and tFLT3 levels, either a separate population of bone marrow samples must be tested and different groupings made based on altered distribution and altered signal range (likely higher given the greater percentage of blast found in bone marrow compared to blood), or if some mathematical relationship was determined between bone marrow signals and blood signals, the above-listed blood signals may be adjusted and used for blood marrow samples. Furthermore, a statistical analysis may be made of the distribution in order to arrive at a more rigorous grouping (or binning) of “low”, “medium” and “high” levels of pFLT3, and correlations and predictions may be made based on those groupings. Alternatively, the grouping or binning may be made into two categories “low” and “high” for generating binary predictions.

Correlation Between FLT3/pFLT3 Levels and Blast Count:

Table 5 shows the distribution of the patients with low, medium and high blast counts among low, medium and high tFLT3 signals:

TABLE 5

| | Low | Medium | High | |
|---|---|---|---|---|
| | tFlt3 | tFlt3 | tFlt3 | Total |
| PB blast count | (<10K) | (10K-50K) | (>50K) | samples |
| Low blast count (<1) | 10 | 6 | 2 | 18 |
| Medium blast count (1-10) | 4 | 5 | 4 | 13 |
| High blast count (>10) | 0 | 6 | 8 | 14 |
| Total | 14 | 17 | 14 | 45 |

Table 6 shows the distribution of the patients with low, medium and high blast counts among low, medium and high pFLT3 signals:

TABLE 6

| | Low | Medium | High | |
|---|---|---|---|---|
| | pFlt3 | pFlt3 | pFlt3 | Total |
| PB blast count | (<500) | (500-1000) | (>1000) | samples |
| Low blast count (<1) | 15 | 1 | 2 | 18 |
| Medium blast count (1-10) | 6 | 6 | 1 | 13 |
| High blast count (>10) | 3 | 0 | 11 | 14 |
| Total | 24 | 7 | 14 | 45 |

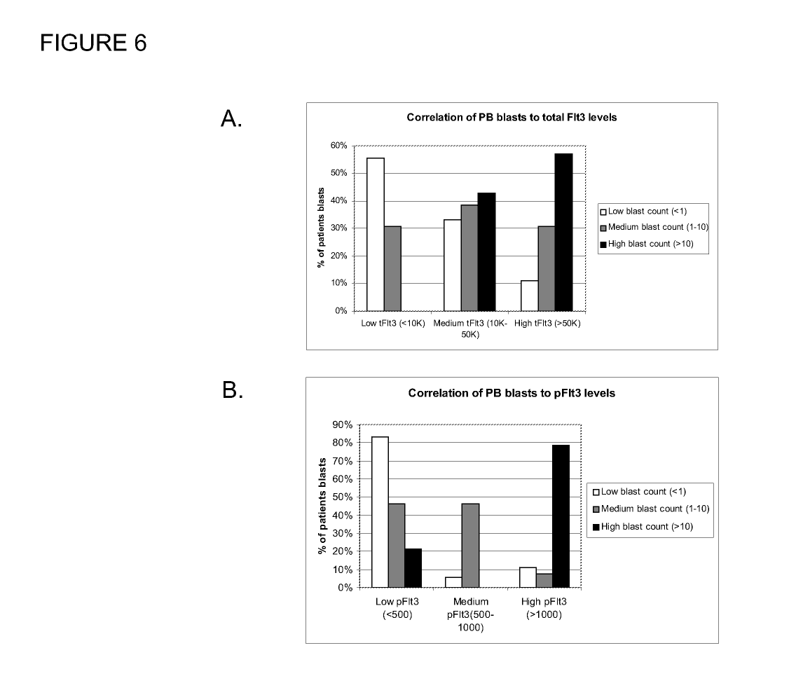

Of the 51 patients in the study, 45 samples were tested, the following correlations were established. FIG. 6A shows the correlation between absolute blast count (number of blast cells in 1000/μl and FLT3 expression among 45 clinical patient undergoing AC220 treatment. Based on the distribution of the signals across 45 plasma samples and the strength of the MSD ELISA signal, a signal of less than 10,000 was designated low level of tFLT3, signals equal to or greater than 10,000 and less than 50,000 was designated medium level of tFLT3 and signals equal to or greater than 50,000 was designated high level of tFLT3. Spearman's rho non-parametric analysis among a sample set of 44 XY pairs yielded a Spearman rho of 0.5216, indicating a significant correlation between blast count and pFLT3 (p=0.0003). A definite correlation trend may be observed, with zero high blast count patient samples exhibiting low total FLT3 levels, whereas about 42% and 55% of patient blasts exhibit, respectively, medium and high levels of tFLT3. A similar trend may be observed for low blast count patient samples, with only about 10% of low blast count patient samples exhibiting high tFLT3 whereas about 33% and 55% of patient blast samples, respectively, exhibit medium and low tFLT3.

FIG. 6B shows the correlation between blast count and pFLT3 levels among the same 45 clinical patient analyzed for pFLT3. Based on the distribution of the signal across 45 plasma samples and the strength of the MSD ELISA signal, a signal of less than 500 was designated low level of pFLT3, signals equal to or greater than 500 and less than 1,000 was designated medium level of pFLT3 and signals equal to or greater than 1,000 was designated high level of pFLT3. Spearman's rho non-parametric analysis yielded a Spearman rho of 0.5196, indicating a significant correlation between blast count and pFLT3 (p=0.0003). In looking at the distribution of the three groupings of blast count against distribution among the three groupings based on pFLT3 expression, the correlation between blast count and pFLT3 levels is even more striking than the correlation between blast count and tFLT3, with almost 80% of high blast count patient samples exhibiting a high level of pFLT3, and conversely, with greater than 80% low blast count patient samples exhibiting low levels of pFLT3.

Shows the pFlt3 inhibition correlates well with clinical outcome. AC220 is also shown to have the greatest impact on ITD patients with constitutively active pFLT3—as would be expected of a FLT3 inhibitor.

Based on the 80% correlation between high pFLT3 levels and high blast count observed among 45 samples, pFLT3 levels will clearly be useful as an independent biomarker for disease status. Assuming that the number of samples (n=45) is sufficient to provide a statistically significant correlation, and assuming that the groupings of low (MSD signal of less than 500), medium (MSD signal equal to or greater than 500 and less than 1000) and high (MSD signal equal to or greater than 1000) are statistically rigorous groupings, the in vivo FLT3 MSD ELISA assay may predict the following:

1) The probability that the absolute blast count is greater than 10 (×1000/μL) given that the pFLT3 level is “high”=11/14 or 79%.

2) The probability that the absolute blast count is greater than 10 (×1000/μL) given that the pFLT3 level is “medium”=0/7 or 0%.

3) The probability that the absolute blast count is greater than 10 (×1000/μL) given that the pFLT3 level is “low”=3/24 or 13%.

Therefore, making the assumptions stated earlier, probabilities of 79%, and 13% of the absolute blast count being greater than 10 (×1000/μL) may be assigned to a patient blood sample generating “high” or “low” pFLT3 levels based on the MSD signal.

Thus, the MSD ELISA assays disclosed herein have the potential to be a more accurate measure of blast count or used as a surrogate for blast count because it is a quantitative biomolecular assay that is less prone to technician error or variability in contrast to the traditional method of counting blast, which consists of counting the number of myeloblasts as a fraction of all nonerythroid nucleated cells present in a smear observed under the microscope.

Correlation between FLT3/pFLT3 levels and FLT3-ITD status:

Table 7 shows the distribution of the genotyped patients among low, medium and high FLT3 signals.

TABLE 7

| | Low | Medium | High | |
|---|---|---|---|---|
| Flt3 | tFlt3 | tFlt3 | tFlt3 | Total |
| genotype | (<10K) | (10K- | (>50K) | samples |
| w.t. | 6 | 7 | 6 | 19 |
| ITD | 1 | 7 | 3 | 11 |
| TKD mut | 2 | 0 | 2 | 4 |
| Total | 9 | 14 | 11 | 34 |

Table 8 shows raw data from an in vivo pFLT3 MSD ELISA and distribution of the genotyped patients among low, medium and high pFLT3 signals.

TABLE 8

| | Low | Medium | High | |
|---|---|---|---|---|
| Flt3 | % pFlt3 | % pFlt3 | % pFlt3 | Total |
| genotype | (<2%) | (2-5%) | (>5%) | samples |
| w.t. | 8 | 6 | 3 | 17 |
| ITD | 2 | 4 | 5 | 11 |
| TKD mut | 1 | 2 | 0 | 3 |
| Total | 11 | 12 | 11 | 34 |

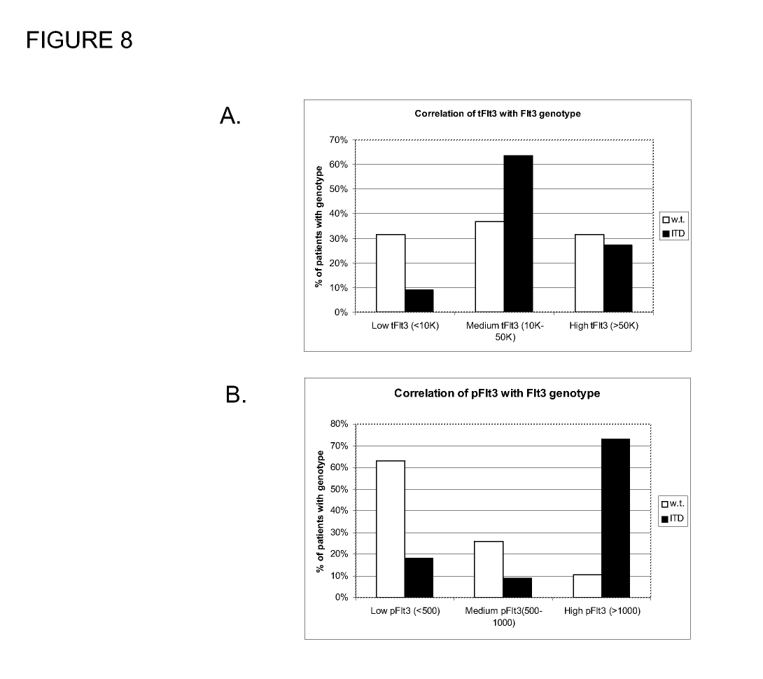

FIG. 8 is a graph showing correlation between genotype and FLT3 levels based on MSD ELISA data obtained from 34 samples from patients receiving AC220 therapy whose genotype were established. FIG. 8A shows a good correlation between genotype and tFLT3 levels, with almost 90% of FLT3-ITD patients expressing medium to high levels of tFLT3. In contrast, wild type patients show a wide range of FLT3 expression. There is an even stronger correlation between FLT3-ITD genotype and high pFLT3 expression, with 73% of FLT3-ITD patients exhibiting high pFLT3 levels. There is a strong converse correlation between low pFLT3 and wild type status, with 63% of wild type patients exhibiting low levels of pFLT3.

Assuming that the number of samples (n=34) is sufficient to provide a statistically significant correlation, and assuming that the groupings of low (MSD signal of less than 500), medium (MSD signal equal to or greater than 500 and less than 1000) and high (MSD signal equal to or greater than 1000) are statistically rigorous groupings, the in vivo FLT3 MSD ELISA assay may predict the following:

1) The probability that the genotype is ITD given that the pFLT3 level is “high”=8/11 or 73%.

2) The probability that the genotype is ITD given that the pFLT3 level is “medium”=1/6 or 17%.

3) The probability that the genotype is ITD given that the pFLT3 level is “low”=2/17 or 12%.

4) The probability that ITD is positive regardless of pFLT3 level for n=34=11/34, or 32%.

Therefore, making the assumptions stated earlier, probabilities of 73%, 17% and 12% of a the ITD genotype being present may be assigned to a patient blood sample generating, respectively, “high”, “medium” and “low” pFLT3 levels based on the MSD signal. Given the modest sample size of n=34, the last probability prediction, that 30% of patients are ITD regardless of pFLT3 levels, agrees very well with the literature estimating 30% of all AML patients as being FLT3-ITD positive.

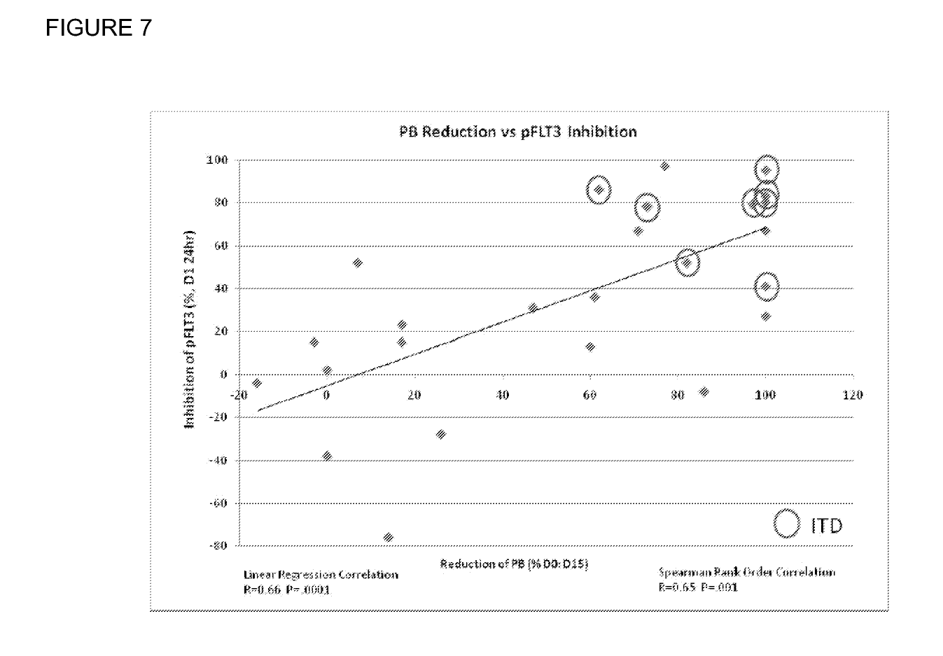

The methods disclosed herein have produced evidence to support hypotheses that would be difficult to confirm in the absence of a sensitive detection method for pFLT3. For example, the hypothesis among leukemia researchers that ITD mutants will respond better to FLT3 inhibitors is based on the assumption that ITD patients have high levels of pFlt3. The data in FIG. 7, obtained via methods of the invention, show a correlation between pFLT3 to FLT3 genotype which supports that assumption. The data show that approximately 75% of ITD patients in the current sample set exhibit high pFLT3 levels compared to wild type patients, only 10% exhibit high pFLT3. The methods disclosed herein also provide for the first time a measure of the percentage of wild type patients expresssing high levels of FLT3. Because only about 30% of adult AML patients bear the FLT3-ITD mutation, and since the FLT3 tyrosine kinase domain mutations such as the D835 point mutation account for another 7% of AML patients, it has been speculated that the remaining AML patients are wild type patients either overexpresssing the FLT3 receptor or overexpressing the FLT3 ligand, or both. The graph in FIG. 6 shows that, in fact, only about 30% of wild type patient exhibit high tFLT3 levels and of those, only 10% exhibit activated pFLT3, which suggests that other mechanisms might be at play in giving rise to AML.

### Example 9

### Patient Monitoring for pFLT3 and Total FLT3 Levels in a Patient Following Treatment with FLT3 Inhibitor

The assays are optimized to monitor pFLT3 and total FLT3 levels in patients following treatment of FLT3 inhibitors as described above. Briefly, patients with AML or other FLT3-mediated diseases are administered with a FLT3 inhibitor, such as AC220. Blood samples are taken at specific time points over the course of a 24 hour period following administration of the FLT3 inhibitor. The samples may be tested retrospectively in a single ELISA plate, as was done in Example 2, or the samples may be tested in real time using fresh blood or bone marrow samples. The preparation of blood sample and detection of total and phosphorylated FLT3 are carried out as in Example 2.

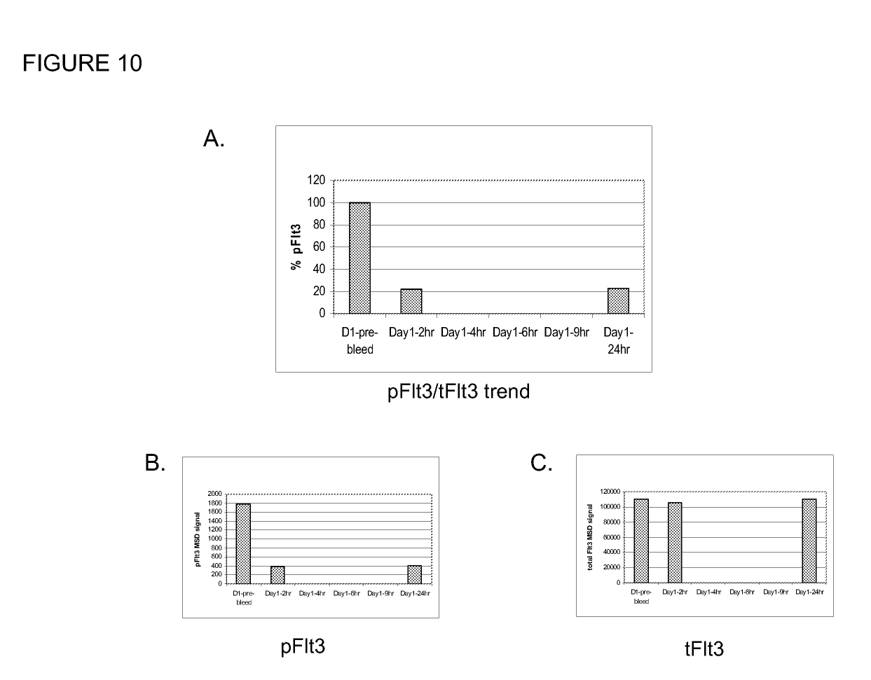

Patient E is one of 51 patients whose blood samples were tested using the MSD ELISA described in Example 2. FIGS. 10B and 10C show the raw data generated for Patient E, who was determined to be an ITD patient. The sample drawn from patient E generated a high MSD signal of about 1800, as would be expected for an ITD patient. FIG. 10A shows the trend in the ratio of pFLT3/tFLT3 at the predose and 2 hour and 24 hour post dose time points. This patient shows a rapid response to AC220 therapy, with the pFLT3/tFLT3 ratio dropping by 80% in only two hours. Rapidity of response is very likely an indication that Patient E is chemosensitive to FLT3 therapy and as rapid response would predict, this patient met the protocol defined response of CRi (complete response with incomplete blood count recovery).

The data for Patient E was generated retrospectively, but the results show how the MSD ELISA methods of the invention may be used to monitor patients and manage their FLT-3 mediated disease by testing samples in real time and making treatment decisions based on the results. For example, a newly diagnosed leukemia patient may have his blood drawn and measured for pFLT3 and tFLT3 and if the MSD ELISA signal of this patient was at 1,800, the treating physician may refer to a scoring system in which MSD ELISA signals were assigned percent probabilities of the presence of an ITD mutation. In this case, the physician may conclude that the patient has a greater than 1000 MSD signal level and is therefore a “high” pFLT3 expressing patient and using the scoring similar to that described in Example 8, the physician may conclude that this patient had a 73% probability of being an ITD patient. The physician may then choose a course of therapy for this patient, and prescribe a targeted FLT3 inhibitor such as AC220. Following administration of the targeted FLT3 therapy, the physician may then choose to take a two hour post dose blood sample and assess the patient's response to FLT3 therapy. If this patient exhibits the type of rapid response shown by Patient E, the physician may choose to continue the target FLT3 inhibition therapy. If the patient does not show the type of rapid response exemplified by Patient E, the physician may choose either choose to stop the treatment or continue treatment and take a third time point either at the end of week or possibly at the end of two weeks, to determine whether the pFLT3 levels were inhibited at a later point in the treatment Inhibition or lack of pFLT3 inhibition at the third time point may guide the physician in determining whether the targeted FLT3 therapy was appropriate for the patient.

### Example 10

### FLT3 MSD ELISA for Testing pFLT3 Inhibition in Bone Marrow Aspirates

The FLT3 and pFLT3 assays are conducted essentially as described in Example 2, except bone marrow aspirates in varying amounts are added to each well instead of blood. Bone marrow aspirates or other bodily fluids are taken from patients at specific time points following administration of FLT3 inhibitors or over the course of treatment. The specimens are lysed with cell lysis buffer supplemented with protease and phosphatase inhibitors and then stored at −80° C. until direct analysis by ELISA, or alternatively, stored directly at −80° C. and lysed with cell lysis buffer supplemented with protease and phosphatase inhibitors before direct analysis by ELISA.

### Example 11

### FLT3 MSD ELISA for Testing pFLT3 Inhibition in Other Bodily Fluid and Tissue Samples

The FLT3 and pFLT3 assays are conducted essentially as described in Example 2, except spinal cord fluid is added in varying amounts are added to each well instead of blood. Alternatively, sample tissue, such as brain, lymph nodes, spleen, skin, or samples from the GI tract that is homogenized or otherwise prepared (e.g., extracts) by methods known in the art are added in varying amounts to each well instead of blood. For example, the tissue sample is thawed and homogenized in lysis buffer using a homogenizer. The homogenized sample is then cleared by centrifugation and the lysate is added to a well of the multi-well plate. The samples can be taken from patients following administration of FLT3 inhibitors or at any time during the course of treatment to monitor the status of pFLT3. The samples are stored at −80° C. until analysis.

### Example 12

### Screening and Identifying Agonists for FLT3 Phosphorylation

The assays described above, e.g., in Example 3, are modified to screen and identify small molecules that are agonists of FLT3 phosphorylation. Different test compounds are prepared as a 2× stock in 0.5% FBS media in a total volume of 250 μl each. 100 μl of 2× stock test compound is then added to the 100 μl of cells in the 96-well plate. A 9 point, 3× dose titration is used for each of the test compound. The cells are incubated with the compound for 2 hours at 37° C., and the assay is run as otherwise described above in Example 3. The signals of pFLT3 are normalized with total FLT3 to account for the difference in cell numbers in different wells. The EC₅₀ of tested compounds is determined from a dose-response curve that shows the increase of FLT3 phosphorylation at various concentrations of tested compounds compared to control samples.

### Example 13

### Screening and Identifying Antagonists for FLT3 Phosphorylation

The assays described herein are modified to screen and identify small molecules that are antagonists of FLT3 phosphorylation. The protocol is carried out essentially as described in Example 12. The signals of pFLT3 are normalized with total FLT3 to account for the difference in cell numbers in different wells. The inhibition of FLT3 is determined from a dose-response curve that shows the inhibition of phosphorylation of FLT3 in the presence of FLT3 inhibitors at various concentrations compared to control samples.

### Example 14

### Using pFLT3 Assay Results to Guide Drug Dosing

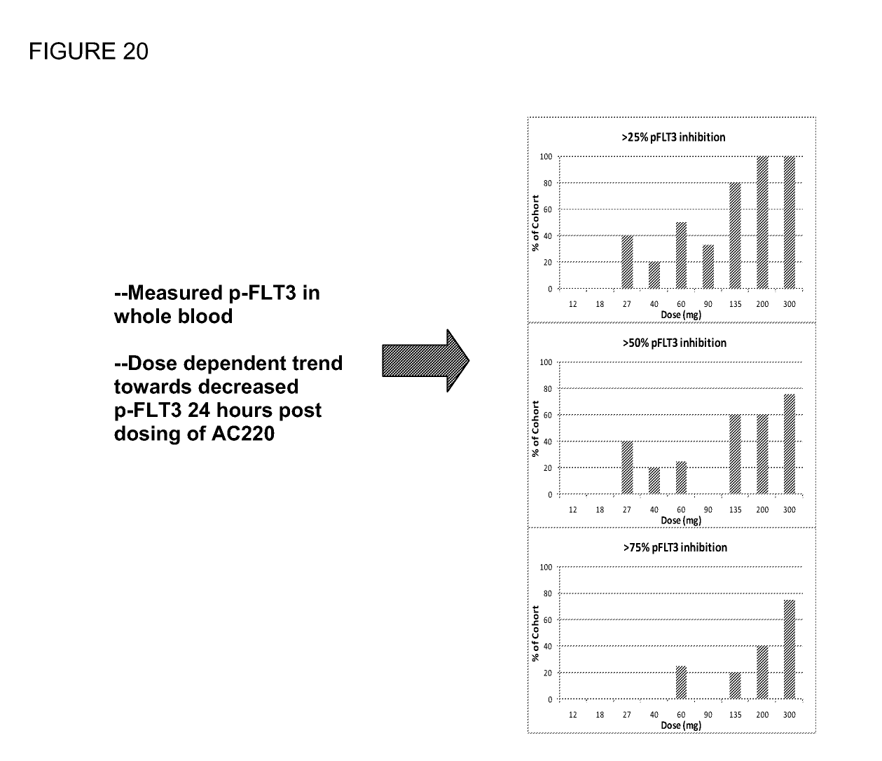

The pFLT3 ELISA method of the invention performed on the blood samples collected from the AC220 clinical study, performed as described in Example 2, were successfully used to monitor the extent of inhibition of FLT3 phosphorylation in the clinical patients. FIG. 20 is a summary of the percentage of patients in each cohort (the x-axis representing the AC220 dose at each cohort) exhibiting either greater than 25% (top graph), greater the 50% (center graph) or greater than 75% reduction (bottom graph) in pFLT3 compared to initial prebleed levels of pFLT3. Because AC220 was well tolerated up to 300 mg per day, QD (once daily), on the 14 days on/14 days off intermittent therapy with no dose limiting toxicity observed, and furthermore, based on the observation from the pharmacodynamics data from the pFLT3 ELISA showing a rebound in pFLT3 levels during the 14 day rest period, the decision was made to change the AC220 dosing schedule from a 14 days on/14 days off intermittent schedule, to a 28-day continuous schedule. Because the maximum tolerated dose had not been reached at 300 mg per day QD, the data shown in FIG. 20 was used to select the starting continuous dose at 200 mg per day QD based on the observation that significant reduction in FLT3 phosphorylation, i.e., 75% or greater reduction, was observed in a significant percentage of patients starting at the 200 mg per day cohort. The bottom graph of FIG. 20 shows that twice as many patients in the 200 mg per day cohort responded with greater than 75% pFLT3 reduction compared to the cohort immediately below, dosed at 135 mg per day. Thus, the pFLT3 ELISA of the invention successfully yielded useful pharmacodynamic data that not only guided the decision to change the dosing schedule from intermittent to continuous, but also guided the selection of the starting dose level for the 28-day continuous schedule, when information on dose limiting toxicity or maximum tolerated dose was still unavailable.

The embodiments of the present invention described above are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims. Furthermore, as used in this specification and claims, the singular forms “a,” “an” and “the” include plural forms unless the content clearly dictates otherwise. Thus, for example, reference to “an antibody” includes a mixture of two or more such antibodies, and the like. Additionally, ordinarily skilled artisans will recognize that operational sequences must be set forth in some specific order for the purpose of explanation and claiming, but the present invention contemplates various changes beyond such specific order.

The contents of all references described herein are hereby incorporated by reference.

Other embodiments are within the following claims.

1. A method for detecting the presence of human phosphorylated FMS-related tyrosine kinase 3 (pFLT3) in a blood, blood lysate or bone marrow aspirate sample, said method comprising:
(a) contacting the sample with an immobilized first antibody that immunospecifically binds to human total FLT3;
(b) removing unbound sample;
(c) contacting the sample bound to the immobilized first antibody with a detectable second antibody, wherein the second antibody immunospecifically binds to human pFLT3, and wherein the second antibody immunospecifically binds to a different FLT3 epitope than the first antibody;
(d) removing unbound second antibody; and
(e) detecting the presence of second antibody bound to the sample;
wherein an increase in the amount of second antibody bound to the sample, as compared to a control sample lacking human pFLT3, indicates the presence of human pFLT3 in the sample.

2. A method for detecting FLT3 phosphorylation in a blood, blood lysate or bone marrow aspirate, said method comprising:
(a) contacting the sample with an immobilized first antibody that immunospecifically binds to human total FLT3;
(b) removing unbound sample;
(c) contacting the sample bound to the immobilized first antibody with a detectable second antibody, wherein the second antibody immunospecifically binds to human pFLT3, and wherein the second antibody immunospecifically binds to a different epitope than the first antibody;
(d) removing unbound second antibody; and
(e) detecting the presence of the second antibody bound to the sample;
wherein an increase in the amount of second antibody bound to the sample, as compared to a control sample having no pFLT3, indicates FLT3 phosphorylation in the sample.

3. A method for diagnosing a patient having a FLT3-activating mutation, said method comprising:
(a) contacting a blood, blood lysate or bone marrow aspirate sample from the patient with an immobilized first antibody that immunospecifically binds to human total FLT3;
(b) removing unbound sample;
(c) contacting the sample bound to the immobilized first antibody with a detectable second antibody, wherein the second antibody immunospecifically binds to human pFLT3, and wherein the second antibody immunospecifically binds to a different FLT3 epitope than the first antibody;
(d) removing unbound second antibody; and
(e) detecting the presence of second antibody bound to the sample.

4. A method for identifying a test compound that activates or is otherwise an agonist of human FLT3 phosphorylation, said method comprising:
(a) contacting a blood, blood lysate or bone marrow aspirate sample comprising human FLT3 in the presence and absence of the test compound;
(b) contacting the sample with an immobilized first antibody that immunospecifically binds to human total FLT3;
(c) removing unbound sample;
(d) contacting the sample bound to the immobilized first antibody with a detectable second antibody, wherein the second antibody immunospecifically binds to human pFLT3, and wherein the second antibody immunospecifically binds to a different FLT3 epitope than the first antibody;
(e) removing unbound second antibody; and
(f) detecting the presence of second antibody bound to the sample;
wherein an increase in the amount of second antibody bound to the sample in the presence of the test compound, as compared to the amount of second antibody bound to the sample in the absence of the test compound, indicates the test compound activates human FLT3 phosphorylation.

5. A method for identifying a test compound that inhibits or is otherwise an antagonist of human FLT3 phosphorylation, said method comprising:
(a) contacting a sample comprising human FLT3 in the presence and absence of the test compound;
(b) contacting the sample with an immobilized first antibody that immunospecifically binds to human total FLT3;
(c) removing unbound sample;
(d) contacting the sample bound to the immobilized first antibody with a detectable second antibody, wherein the second antibody immunospecifically binds to human pFLT3, and wherein the second antibody immunospecifically binds to a different FLT3 epitope than the first antibody;
(e) removing unbound second antibody; and
(f) detecting the presence of second antibody bound to the sample;
wherein a decrease in the amount of second antibody bound to the sample in the presence of the test compound, as compared to the amount of second antibody bound to the sample in the absence of the test compound, indicates the test compound inhibits human FLT3 phosphorylation.

6. The method of claim 4 or 5, wherein the test compound is one of a plurality of test compounds, wherein at least two of the test compounds differ from one another.

7. The method of claim 6, wherein the plurality of test compounds comprises between 1 and 100,000 test compounds, between 1 and 35,000 test compounds, between 1 and 10,000 test compounds, between 1 and 1,000 test compounds, between 1 and 100 test compounds, or between 1 and 10 test compounds.

8. A method for determining the efficacy of a compound for decreasing human FLT3 phosphorylation in a patient, said method comprising:
(a) administering the compound to the patient;
(b) contacting a blood, blood lysate or bone marrow aspirate sample from the patient with an immobilized first antibody that immunospecifically binds to human total FLT3;
(c) removing unbound sample;
(d) contacting the sample bound to the immobilized first antibody with a detectable second antibody, wherein the second antibody immunospecifically binds to human pFLT3, and wherein the second antibody immunospecifically binds to a different FLT3 epitope than the first antibody;
(e) removing unbound second antibody; and
(f) detecting the presence of second antibody bound to the sample;
wherein a decrease in the amount of second antibody bound to the sample, as compared to a control sample from the patient prior to administration of the compound, indicates the efficacy of the compound for decreasing human FLT3 phosphorylation in the patient.

9. The method of claim 1, wherein the sample is from a patient.

10. The method of claim 9, wherein the patient has a FLT3-activating mutation.

11. The method of claim 1, wherein the second antibody comprises a label.

12. The method of claim 11, wherein the label is a biotin, radionuclide, enzyme, substrate, fluorescent marker, chemiluminescent marker or ruthenium (II) tri-bipyridine-(4-methylsulfonate) NHS ester.

13. The method of claim 1, wherein the first antibody is immobilized in a well of a multi-well or multi-domain plate.

14. The method of claim 13, wherein the multi-well or multi-domain plate comprises electrodes on the bottom of the plate.

15. The method of claim 13, wherein the second antibody is biotinylated.

16. The method of claim 15, further comprising contacting the second antibody with labeled-streptavidin and removing unbound labeled-streptavidin after removing unbound second antibody and before detecting the presence of second antibody bound to the sample.

17. The method of claim 16, wherein the labeled-streptavidin comprises a ruthenium (II) tri-bipyridine-(4-methylsulfonate) NHS ester.

18. The method of claim 17, wherein the presence of second antibody bound to the sample is detected by ECL of the tagged-streptavidin on the surface of the carbon electrodes.

19. A method for detecting the presence of human pFLT3 in a sample, said method comprising:
(a) contacting the sample with an immobilized first antibody that immunospecifically binds to the extracellular domain of human FLT3, wherein the first antibody is immobilized in a well on a multi-well plate or multi-domain multi-well plate comprising electrodes on the bottom of the plate;
(b) removing unbound sample;
(c) contacting the sample bound to the immobilized first antibody with a mixture of a biotinylated second antibody and a labeled-streptavidin, wherein the second antibody immunospecifically binds to human pFLT3, and wherein the second antibody immunospecifically binds to a different FLT3 epitope than the first antibody and wherein the labeled-streptavidin comprises ruthenium (II) tri-bipyridine-(4-methylsulfonate) NHS ester;
(d) removing unbound second antibody and unbound labeled-streptavidin; and
(e) detecting the presence of second antibody bound to the sample by ECL of the labeled-streptavidin on the surface of the electrodes;
wherein a greater amount of second antibody bound to the sample, as compared to a control sample either lacking human pFLT3 or having an undetectable amount of human pFLT3, indicates the presence of human pFLT3 in the sample.

20. The method of claim 1, wherein the immobilized first antibody is added to the well at a concentration ranging from about 0.1 μg/mL to about 10 μg/mL.

21. The method of claim 1, wherein about 0.25 ng to about 2.5 μg, or about 2.5 ng to about 250 ng of first antibody is added per well.

22. The method of claim 1, wherein the first antibody immunospecifically binds to the extracellular domain of human FLT3 27-543.

23. The method of claim 1, wherein the first antibody immunospecifically binds to the native form of human FLT3.

24. The method of claim 1, wherein the first antibody does not immunospecifically bind to one or more of the juxtamembrane domain (amino acids 572-603 SEQ ID NO:1), kinase insert region amino acids 711-780 of SEQ ID NO:1, intracellular domain amino acids 564-993 of SEQ ID NO:1 and C-terminus (amino acids 974-993 of SEQ ID NO:1).

25. The method of claim 1, wherein the first antibody is not a polyclonal antibody.

26. The method of claim 1, wherein the first antibody is a murine monoclonal antibody.

27. The method of claim 1, wherein the second antibody immunospecifically binds to one or more of the phosphorylated tyrosine residues 589, 591, 597, 599, 726, 842 and 955 of pFLT3.

28. The method of claim 1, wherein the second antibody does not immunospecifically bind solely to one or more of the phosphorylated tyrosine residues 589 and 591.

29. The method of claim 1, wherein the second antibody is a murine monoclonal antibody.

30. The method of claim 1, wherein the second antibody is contacted with the sample at a concentration ranging from about 0.1 μg/mL to about 10 μg/mL.

31. The method of claim 1, wherein about 0.25 ng to about 2.5 μg, or about 2.5 ng to about 250 ng of second antibody is added per well.

32. A kit for carrying out the method of claim 1, in one or more containers.

33. A kit for detecting the presence of human pFLT3 in a sample, said kit comprising:
(a) a multi-well plate or multi-domain plate comprising electrodes on the bottom of the plate;
(b) a first antibody that immunospecifically binds to the extracellular domain of human FLT3, wherein the first antibody is optionally immobilized in a well of the plate;
(c) a second antibody, wherein the second antibody immunospecifically binds to human pFLT3, and wherein the second antibody immunospecifically binds to a different FLT3 epitope than the first antibody, and wherein the second antibody is optionally biotinylated; and
(d) optionally, a labeled-streptavidin comprises ruthenium (II) tri-bipyridine-(4-methylsulfonate) NHS ester.

34. The kit of claim 33, further comprising necessary reagents, and optionally, positive and negative controls.


SEQUENCE LISTING (1 sequence)

SEQ ID NO: 1 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS